(19) United States
(12) Patent Application Publication
(10) Pub. No.: US 2022/0017566 A1
(43) Pub. Date: Jan. 20, 2022

(54) TERPINOID DERIVATIVES AND USES THEREOF

(71) Applicant: SICHUAN HAISCO PHARMACEUTICAL CO., LTD., Wenjiang District Chengdu, Sichuan (CN)

(72) Inventors: Bohan JIN, San Diego, CA (US); Qing DONG, San Diego, CA (US); Gene HUNG, San Diego, CA (US); Stephen W. KALDOR, San Diego, CA (US)

(21) Appl. No.: 17/280,824

(22) PCT Filed: Sep. 23, 2019
(86) PCT No.: PCT/US2019/052472

§ 371 Date: Mar. 26, 2021

Related U.S. Application Data
(60) Provisional application No. 62/738,762, filed on Sep. 28, 2018.
(60) Provisional application No. 62/770,569, filed on Nov. 21, 2018.
(60) Provisional application No. 62/808,192, filed on Feb. 20, 2019.
(60) Provisional application No. 62/823,846, filed on Mar. 26, 2019.

(51) Int. Cl. C07J 63/00

(52) U.S. Cl. CPC C07J 63/008

(57) ABSTRACT

Described herein are terpinoid derivatives as NRF2 inhibitors and pharmaceutical compositions comprising said compounds. The subject compounds and compositions are useful for the treatment of inflammatory diseases.

### CROSS-REFERENCE

This patent application claims the benefit of U.S. Provisional Application No. 62/738,762, filed Sep. 28, 2018; U.S. Provisional Application No. 62/770,569, filed, Nov. 21, 2018; U.S. Provisional Application No. 62/808,192, filed, Feb. 20, 2019; and U.S. Provisional Application No. 62/823,846, filed Mar. 26, 2019; each of which is incorporated herein by reference in their entirety.

### FIELD OF THE INVENTION

Described herein are compounds, methods of making such compounds, pharmaceutical compositions and medicaments comprising such compounds, and methods of using such compounds to treat, prevent or diagnose diseases, disorders, or conditions associated with oxidative stress and inflammation.

### BACKGROUND OF THE INVENTION

The anti-inflammatory and anti-proliferative activity of the naturally occurring triterpenoid, oleanolic acid, has been improved by chemical modifications. For example, 2-cyano-3,12-diooxooleana-1,9(11)-dien-28-oic acid (CDDO) and related compounds have been developed. Bardoxolone methyl (CDDO-Me) is currently being evaluated in phase III clinical trials for the treatment of diabetic nephropathy and chronic kidney disease.

Synthetic triterpenoid analogs of oleanolic acid have also been shown to be inhibitors of cellular inflammatory processes, such as the induction by IFN-y of inducible nitric oxide synthase (iNOS), and of COX-2 in mouse macrophages. Compounds derived from oleanolic acid have been shown to affect the function of multiple protein targets and thereby modulate the activity of several important cellular signaling pathways related to oxidative stress, cell cycle control, and inflammation. Given that the biological activity profiles of known triterpenoid derivatives vary, and in view of the wide variety of diseases that may be treated or prevented with compounds having potent antioxidant and anti-inflammatory effects, and the high degree of unmet medical need represented within this variety of diseases, it is desirable to synthesize new compounds with diverse structures that may have improved biological activity profiles for the treatment of one or more indications.

### BRIEF SUMMARY OF THE INVENTION

Disclosed herein is a compound of Formula (III), or a pharmaceutically acceptable salt, solvate, or stereoisomer thereof:

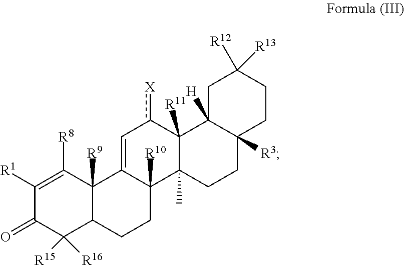

wherein:
R¹ is hydrogen, deuterium, halogen, —CN, —OR^{b}, —NR^{c}R^{d}, —NR^{b}S(═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R³ is N-linked heterocycloalkyl, N-linked heteroaryl, —P(═O)(R⁴)₂, —P(═O)(OR⁵)₂, —B(OR⁵)₂, —S(═O)R⁴, —S(═O)₂R⁴, —NR⁵C(═NR^{x})R⁵, —NR⁵C(=NR^{x})NR⁶R⁷, —S(═O)(═NR^{x})R⁵, —S(═O)(=NR^{x})NR⁶R⁷, —NR⁵S(═O)₂NR⁵C(═O)R⁵, —NR⁵S(═O)(═NR^{x})R⁵, —Z—O-cycloalkyl, —Z—O- heterocycloalkyl, —Z—O-aryl, —Z—O-heteroaryl, —Z—NR⁵-cycloalkyl, —Z—NR⁵-heterocycloalkyl, —Z—NR⁵-aryl, —Z—NR⁵-heteroaryl, —Y(C₁—C₆alkylene)S(═O)R⁴, —Y(C₁C₆alkylene)S(═O)₂R⁴, —Y(C₁-C₆alkylene)P(═O)(R⁴)₂, —Y(C₁—C₆alkylene)P(═O)(OR⁵)₂, —Y(C₁-C₆alkylene)B(OR⁵)₂, —Y(C₁-C₆alkylene)NR⁵C(═NR^{x})R⁵, —Y(C₁-C₆alkylene)NR⁵C(═NR^{x})NR⁶R⁷, —Y(C₁—C₆alkylene)S(═O)(═NR^{x})R⁵, —Y(C₁—C₆alkylene)S(═O)(═NR^{x})NR⁶R⁷, —Y(C₁-C₆alkylene)NR⁵S(═O)₂NR⁵C(═O)R⁵, —NR⁵C(═O)(C₁—C₆alkylene)S(═NR^{x})NR⁶R⁷, —NR⁵C(═O)(C₁- C₆alkylene)S(═NR^{x})R⁵, —Y(C₁—C₆alkylene)NR⁵S(═O)(═NR^{x})R⁵, —Y(C₂-C₆alkenylene)S(═O)₂R⁴, —Y(C₂-C₆alkenylene)P(═O)(R⁴)₂, —Y(C₂-C₆alkenylene)P(═O)(OR⁵)₂, —Y(C₂-C₆alkenylene)B(OR⁵)₂, —Y(C₂-C₆alkenylene)NR⁵C(═NR^{x})R⁵, —Y(C₂-C₆alkenylene)NR⁵C(═NR^{x})NR⁶R⁷, —Y(C₂—C₆alkenylene)S(═O)(═NR^{x})R⁵, —Y(C₂-C₆alkenylene)S(═O)(═NR^{x})NR⁶R⁷, —Y(C₂—C₆alkenylene)NR⁵S(═O)₂NR⁵C(═O)R⁵, —Y(C₂-C₆alkenylene)NR⁵S(═O)(═NR^{x})R⁵, —Y(C₂—C₆alkenylene)cycloalkyl, —Y(C₂-C₆alkenylene)heterocycloalkyl, —Y(C₂-C₆alkenylene)aryl, —Y(C₂—C₆alkenylene)heteroaryl, —(C₁-C₆alkylene)OP(═O)(OR⁵)₂, —(C₁-C₆alkylene)O(C₁—C₆alkylene)OP(═O)(OR⁵)₂, or —(C₁-C₆alkylene)OP(═O)(OR⁵)[N(R⁵)₂]; wherein the alkylene, alkenylene, aryl, heteroaryl, cycloalkyl, and heterocycloalkyl are independently optionally substituted with one, two, or three R^{3a};
each R^{3a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
Y is a bond, —O—, —S—, or —NR^{b}—;
Z is a bond or C₁-C₆alkylene;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{4a};
each R^{4a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;

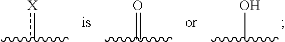

R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R¹¹ are taken together to form an optionally substituted cycloalkyl or an optionally substituted heterocycloalkyl;
R¹² and R¹³ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹² and R¹³ are taken together to form a cycloalkyl or a heterocycloalkyl; each substituted with 0-6 R¹⁴;
each R¹⁴ is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
R¹⁵ and R¹⁶ are independently hydrogen, —OR^{b}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
or R¹⁵ and R¹⁶ are taken together to form a cycloalkyl or a heterocycloalkyl; each substituted with 0-6 R²;
each R² is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

Also disclosed herein is a compound of Formula (VIII), or a pharmaceutically acceptable salt, solvate, or stereoisomer thereof:

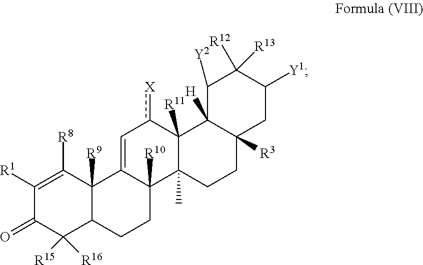

wherein:
R¹ is hydrogen, deuterium, halogen, —CN, —OR^{b}, —NR^{c}R^{d}, —NR^{b}S(═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R³ is halogen, —CN, —OR⁵, —SR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3a};
each R^{3a} is independently oxo, deuterium, halogen, —CN, —OR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3b};
each R^{3b} is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three lea;
each R^{4a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁—C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR⁸)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(=NR⁸)R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;

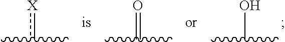

Y¹ and Y² are independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R¹¹ are taken together to form an optionally substituted cycloalkyl or an optionally substituted heterocycloalkyl;
R¹² is hydrogen, deuterium, —OR^{b}, —NR^{c}R^{d}, C₁-C₆ alkyl, C₂-C₆ alkenyl, C₂-C₆ alkynyl, C₁-C₆ haloalkyl, C₁—C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R¹³ is —CN, —OR¹⁹, —S(═O)₂NR²⁰R²¹, —OC(═O)R¹⁸, —OC(═O)OR¹⁹, —OC(═O)NR²⁰R²¹, —NR¹⁹C(═O)OR¹⁹, C₁-C₆ deuteroalkyl, C₁-C6 hydroxyalkyl, C₁-C₆ aminoalkyl, C₁-C₆ heteroalkyl, cycloalkyl, heterocycloalkyl, aryl, heteroaryl, —CH₂(cycloalkyl), —CH₂(heterocycloalkyl), —CH₂(aryl), or —CH₂(heteroaryl); wherein the alkyl, cycloalkyl, heterocycloalkyl, aryl, and heteroaryl are independently optionally substituted with one, two, or three R^{13a};
each R^{13a} is independently oxo, deuterium, halogen, —CN, —OR¹⁹, —SR¹⁹, —S(═O)R¹⁸, —S(═O)₂R¹⁸, —NO₂, —NR²⁰R²¹, —S(═O)₂NR²⁰R²¹, —C(═O)R¹⁸, —OC(═O)R¹⁸, —C(═O)OR¹⁹, —OC(═O)OR¹⁹, —C(═O)NR²⁰R²¹, —OC(═O)NR²⁰R²¹, —NR¹⁹C(═O)OR¹⁹, C₁-C₆ alkyl, C₂-C₆ alkenyl, C₂-C₆ alkynyl, C₁-C₆ haloalkyl, C₁- C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R¹⁸ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{18a};
each R^{18a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R¹⁹ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{19a};
each R^{19a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R²⁰ and R²¹ are independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R²⁰;
each R^{20a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C6 aminoalkyl, C₁-C6 heteroalkyl, cycloalkyl, heterocycloalkyl, aryl, or heteroaryl;
or R²⁰ and R²¹ are taken together with the nitrogen atom to which they are attached to form a heterocycloalkyl optionally substituted with one, two, or three R^{20b};
each R^{20b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R¹⁵ and R¹⁶ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹⁵ and R¹⁶ are taken together to form a cycloalkyl or a heterocycloalkyl; each substituted with 0-6 R²;
each R² is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c}and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

Disclosed herein is a pharmaceutical composition comprising a therapeutically effective amount of a compound disclosed herein, and a pharmaceutically acceptable excipient.

Also disclosed herein is a method for treating a disease in a mammal comprising administering to the mammal a therapeutically effective amount of a compound or a pharmaceutical composition disclosed herein. In some embodiments, the disease is an inflammatory disease. In some embodiments, the disease is diabetic nephropathy or chronic kidney disease. In some embodiments, the disease is chronic obstructive pulmonary disease (COPD) or inflammatory bowel disease (IBD). In some embodiments, the disease is nonalcoholic steatohepatitis (NASH).

### INCORPORATION BY REFERENCE

All publications, patents, and patent applications mentioned in this specification are herein incorporated by reference for the specific purposes identified herein.

### DETAILED DESCRIPTION OF THE INVENTION

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Aliphatic chain” refers to a linear chemical moiety that is composed of only carbons and hydrogens. In some embodiments, the aliphatic chain is saturated. In some embodiments, the aliphatic chain is unsaturated. In some embodiments, the unsaturated aliphatic chain contains one unsaturation. In some embodiments, the unsaturated aliphatic chain contains more than one unsaturation. In some embodiments, the unsaturated aliphatic chain contains two unsaturations. In some embodiments, the unsaturated aliphatic chain contains one double bond. In some embodiments, the unsaturated aliphatic chain contains two double bonds.

“Alkyl” refers to an optionally substituted straight-chain, or optionally substituted branched-chain saturated hydrocarbon monoradical having from one to about ten carbon atoms, or from one to six carbon atoms, wherein an sp^{a}-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein an sp²-hybridized carbon of the alkenyl residue is attached to the rest of the molecule by a single bond. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₃ alkynyl, a C₂—C₇ alkynyl, a C₂-C₆ alkynyl, a C₂-C₅ alkynyl, a C₂-C₄ alkynyl, a C₂-C₃ alkynyl, or a C2 alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

“Aryl” refers to a radical derived from a hydrocarbon ring system comprising hydrogen, 6 to 30 carbon atoms, and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl include, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro, or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxyl)pentyl. In some embodiments, the hydroxyalkyl is hydroxymethyl.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized.

Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3-to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous, and sulfur, and at least one aromatic ring. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

### Compounds

Described herein are triterpenoid derivatives that exhibit, for example, anti-inflammatory and/or antioxidant properties.

Diclosed herein is a compound of Formula (I), or a pharmaceutically acceptable salt, solvate, or stereoisomer thereof:

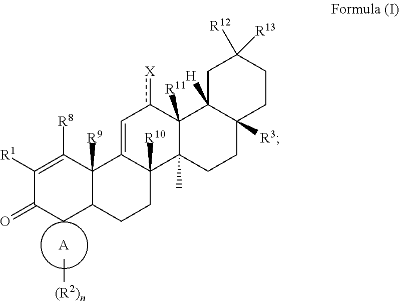

wherein:
Ring A is a cycloalkyl or a heterocycloalkyl;
R¹ is hydrogen, deuterium, halogen, —CN, —OR^{b}, —NR^{c}R^{d}, —NR^{b}S(═O)₂Ra, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
each R² is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
n is 0-6;
R³ is halogen, —CN, —OR⁵, —SR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3a};
each R^{3a} is independently oxo, deuterium, halogen, —CN, —SR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3b};
each R^{3b} is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three lea;
each R^{4a} is independently oxo, deuterium, halogen, —CN, —OR^{b}, —NR⁶R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR⁸)R^{b}, —S(═O)(═NR⁸)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR⁸)R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR⁶C(═NR^{x})NR^{c}R^{d}, —S(═O)(=NR⁸)R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;

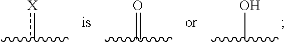

R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R¹¹ are taken together to form an optionally substituted cycloalkyl or an optionally substituted heterocycloalkyl;
R¹² and R¹³ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹² and R¹³ are taken together to form a cycloalkyl or a heterocycloalkyl; each substituted with 0-6 R¹⁴;
each R¹⁴ is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

In some embodiments of a compound of Formula (I), R¹ is —CN, C₁-C₆ alkyl, C₁-C₆ haloalkyl, or C₁-C₆ deuteroalkyl. In some embodiments of a compound of Formula (I), R¹ is —CN or C₁-C₆ haloalkyl. In some embodiments of a compound of Formula (I), W is C₁-C₆ haloalkyl. In some embodiments of a compound of Formula (I), R¹ is —CN.

In some embodiments of a compound of Formula (I), R⁸ is hydrogen or deuterium. In some embodiments of a compound of Formula (I), R⁸ is hydrogen.

In some embodiments of a compound of Formula (I), R⁹ is C₁-C₆ alkyl or C₂-C₆ alkynyl. In some embodiments of a compound of Formula (I), R⁹ is C₁-C₆ alkyl.

In some embodiments of a compound of Formula (I), R¹⁹ is C₁-C₆ alkyl or C₂-C₆ alkynyl. In some embodiments of a compound of Formula (I), R¹⁰ is C₁-C₆ alkyl.

In some embodiments of a compound of Formula (I), R⁹ is C₁-C₆ alkyl and R¹⁰ is C₂-C₆ alkynyl.

In some embodiments of a compound of Formula (I), R¹⁰ is C₁-C₆ alkyl and R⁹ is C₂-C₆ alkynyl.

In some embodiments of a compound of Formula (I),

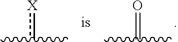

In some embodiments of a compound of Formula (I),

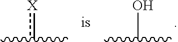

In some embodiments of a compound of Formula (I), R¹¹ is hydrogen, halogen, or —OH. In some embodiments of a compound of Formula (I), R¹¹ is hydrogen or —OH. In some embodiments of a compound of Formula (I), R¹¹ is hydrogen.

In some embodiments of a compound of Formula (I), R¹² and R¹³ are independently hydrogen, C₁-C₆ alkyl, or C₁-C₆ deuteroalkyl. In some embodiments of a compound of Formula (I), R¹² and R¹³ are independently hydrogen or C₁-C₆ alkyl. In some embodiments of a compound of Formula (I), R¹² and R¹³ are independently hydrogen or C₁-C₆ deuteroalkyl. In some embodiments of a compound of Formula (I), R¹² and R¹³ are independently C₁-C₆ alkyl.

In some embodiments of a compound of Formula (I), R¹² and R¹³ are taken together to form a cycloalkyl. In some embodiments of a compound of Formula (I), R¹² and R¹³ are taken together to form a cycloalkyl substituted with 1-4 deuterium. In some embodiments of a compound of Formula (I), R¹² and R¹³ are taken together to form a cycloalkyl substituted with 1 or 2 deuterium. In some embodiments of a compound of Formula (I), R¹² and R¹³ are taken together to form a cycloalkyl substituted with 2-4 deuteriums.

In some embodiments of a compound of Formula (I), R¹² and R¹³ are taken together to form a heterocycloalkyl. In some embodiments of a compound of Formula (I), R¹² and R¹³ are taken together to form a heterocycloalkyl substituted with 1-4 deuterium. In some embodiments of a compound of Formula (I), R¹³ and R¹³ are taken together to form a heterocycloalkyl substituted with 1 or 2 deuterium. In some embodiments of a compound of Formula (I), R¹² and R¹³ are taken together to form a heterocycloalkyl substituted with 2-4 deuteriums.

In some embodiments of a compound of Formula (I), R³ is halogen, —CN, —OR⁵, —NR⁶R⁷, —C(═O)R⁴, —OC(═O)R⁴, —C(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂R⁴, —NR⁵C(═O)R⁴, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₁-C₆ hydroxyalkyl, C₁-C₆ heteroalkyl, or heteroaryl; wherein the alkyl, alkenyl, and heteroaryl are independently optionally substituted with one, two, or three R^{3a}. In some embodiments of a compound of Formula (I), R³ is —NR⁵C(═NR^{x})R⁵ or —NR⁵C(=NR^{x})NR⁶R⁷. In some embodiments of a compound of Formula (I), R³ is —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵.

In some embodiments of a compound of Formula (I), R³ is C₁-C₆ alkyl optionally substituted with one, two, or three R^{3a}. In some embodiments of a compound of Formula (I), R³ is C₁-C₆ alkyl.

In some embodiments of a compound of Formula (I), R³ is —C(═O)OR⁵.

In some embodiments of a compound of Formula (I), each R^{3a} is independently deuterium, halogen, —CN, —OR⁵, —S(═O)R⁴, —S(═O)₂R⁴, —OC(═O)R⁴, —C(═O)OR⁵, —OC(═O)OR⁵, —C(═O)NR⁶R⁷, —OC(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, cycloalkyl, or heterocycloalkyl.

In some embodiments of a compound of Formula (I), each R^{3a} is independently deuterium, halogen, —CN, —OR⁵, —S(═O)R⁴, —S(═O)₂R⁴, —OC(═O)R⁴, —C(═O)OR⁵, —OC(═O)OR⁵, —C(═O)NR⁶R⁷, —OC(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)2NR⁵C(═O)R⁵, or —NR⁵S(═O)(═NR^{x})R⁵. In some embodiments of a compound of Formula (I), each R^{3a} is independently deuterium, halogen, —CN, —OR⁵, —OC(═O)R⁴, —C(═O)OR⁵, —C(═O)NR⁶R⁷, —B(OR⁵)₂, —S(═O)(═NR^{x})R⁵, C₁-C₆ heteroalkyl, heterocycloalkyl, or heteroaryl. In some embodiments of a compound of Formula (I), each R^{3a} is independently —OC(═O)R⁴, —C(═O)OR⁵, —OC(═O)OR⁵, —C(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵. In some embodiments of a compound of Formula (I), each R^{3a} is independently —OC(═O)R⁴, —C(═O)OR⁵, —OC(═O)OR⁵, or —C(═O)NR⁶R⁷. In some embodiments of a compound of Formula (I), each R^{3a} is independently —P(═O)(R⁴)₂, —P(═O)(OR⁵)₂, or —B(OR⁵)₂. In some embodiments of a compound of Formula (I), each R^{3a} is independently —NR⁵C(=NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵.

In some embodiments of a compound of Formula (I), each R^{3b} is independently deuterium, halogen, —CN, —OR^{b}, C₁-C₆ alkyl, C₁-C₆ haloalkyl, or C₁-C₆ deuteroalkyl. In some embodiments of a compound of Formula (I), each R^{3b} is independently deuterium, halogen, C₁-C₆ alkyl, C₁-C₆ haloalkyl, or C₁-C₆ deuteroalkyl.

In some embodiments of a compound of Formula (I), each R⁴ is independently C₁-C₆ alkyl, C₂-C₆ alkenyl, C₂-C₆ alkynyl, C₁-C₆ haloalkyl, C₁-C₆ heteroalkyl, cycloalkyl, or heterocycloalkyl; wherein the alkyl, alkenyl, alkynyl, cycloalkyl, and heterocycloalkyl are independently optionally substituted with one, two, or three R^{4a}. In some embodiments of a compound of Formula (I), each R⁴ is independently C₁-C₆ alkyl or cycloalkyl; wherein the alkyl and cycloalkyl are independently optionally substituted with one, two, or three R^{4a}. In some embodiments of a compound of Formula (I), each R⁴ is independently C₁-C₆ alkyl optionally substituted with one, two, or three R^{4a}. In some embodiments of a compound of Formula (I), each R⁴ is independently C₁-C₆ alkyl. In some embodiments of a compound of Formula (I), each R⁴ is independently C₁-C₆ alkyl, C₁-C₆ haloalkyl, or C₁-C₆ deuteroalkyl.

In some embodiments of a compound of Formula (I), each R^{4a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (I), each R^{4a} is independently deuterium or halogen. In some embodiments of a compound of Formula (I), each R^{4a} is independently halogen.

In some embodiments of a compound of Formula (I), each R⁵ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{5a}. In some embodiments of a compound of Formula (I), each R⁵ is independently hydrogen, C₁-C₆ alkyl, or cycloalkyl; wherein the alkyl and cycloalkyl are independently optionally substituted with one, two, or three R^{5a}. In some embodiments of a compound of Formula (I), each R⁵ is independently hydrogen or C₁-C₆ alkyl optionally substituted with one, two, or three R^{5a}. In some embodiments of a compound of Formula (I), each R⁵ is independently C₁-C₆ alkyl optionally substituted with one, two, or three R^{5a}. In some embodiments of a compound of Formula (I), each R⁵ is hydrogen or C₁-C₆ alkyl. In some embodiments of a compound of Formula (I), each R⁵ is independently hydrogen, C₁-C₆ alkyl, C₁-C₆ haloalkyl, or C₁-C₆ deuteroalkyl.

In some embodiments of a compound of Formula (I), two R⁵ are taken together to form an optionally substituted heterocycloalkyl.

In some embodiments of a compound of Formula (I), each R^{5a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (I), each R^{5a} is independently deuterium or halogen. In some embodiments of a compound of Formula (I), each R^{5a} is independently halogen. In some embodiments of a compound of Formula (I), each R^{5a} is independently —NR^{b}C(═NR^{x})R^{b} or —NR^{b}C(═NR^{x})NR^{c}R^{d}. In some embodiments of a compound of Formula (I), each R^{5a} is independently —S(═O)(═NR^{x})R^{b} or —S(═O)(═NR^{x})NR^{c}R^{d}.

In some embodiments of a compound of Formula (I), each R⁶ and R⁷ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{6a}. In some embodiments of a compound of Formula (I), each R⁶ and IV is independently hydrogen, C₁-C₆ alkyl, C₁-C₆ haloalkyl, cycloalkyl, or heterocycloalkyl; wherein the alkyl, cycloalkyl, and heterocycloalkyl are independently optionally substituted with one, two, or three R^{6a}. In some embodiments of a compound of Formula (I), each R⁶ and R⁷ is independently hydrogen, C₁-C₆ alkyl, or C₁-C₆ haloalkyl; wherein the alkyl is independently optionally substituted with one, two, or three R^{6a}. In some embodiments of a compound of Formula (I), each R⁶ and R⁷ is independently hydrogen or C₁-C₆ alkyl. In some embodiments of a compound of Formula (I), each R⁶ and R⁷ is independently hydrogen, C₁-C₆ alkyl, C₁-C₆ haloalkyl, or C₁-C₆ deuteroalkyl.

In some embodiments of a compound of Formula (I), each R^{6a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (I), each R^{6a} is independently deuterium or halogen. In some embodiments of a compound of Formula (I), each R^{6a} is independently halogen. In some embodiments of a compound of Formula (I), each R^{5a} is independently —NR^{b}C(═NR^{x})R^{b} or —NR^{b}C(═NR^{x})NR^{c}R^{d}. In some embodiments of a compound of Formula (I), each R^{6a} is independently —S(═O)(═NR^{x})R^{b} or —S(═O)(═NR^{x})NR^{c}R^{d}.

In some embodiments of a compound of Formula (I), R⁶ and R⁷ are taken together with the nitrogen atom to which they are attached to form a heterocycloalkyl optionally substituted with one, two, or three R^{6b}.

In some embodiments of a compound of Formula (I), each R^{6b} is independently oxo, deuterium, halogen, —CN, —OR^{b}, —NR^{c}R^{d}, C₁-C₆ alkyl, C₁-C₆ haloalkyl, C₁-C₆ hydroxyalkyl, cycloalkyl, or heterocycloalkyl. In some embodiments of a compound of Formula (I), each R^{6b} is independently C₁-C₆ alkyl or C₁-C₆ haloalkyl. In some embodiments of a compound of Formula (I), each R^{6b} is independently C₁-C₆ alkyl.

In some embodiments of a compound of Formula (I), R^{x} is hydrogen, —NO₂, or —CN. In some embodiments of a compound of Formula (I), R^{x} is —NO₂ or —CN. In some embodiments of a compound of Formula (I), R^{x} is —CN.

In some embodiments of a compound of Formula (I), Ring A is a cycloalkyl. In some embodiments of a compound of Formula (I), Ring A is a heterocycloalkyl.

In some embodiments of a compound of Formula (I), each R² is independently deuterium, halogen, —CN, —OR^{b}, C₁-C₆ alkyl, or C₁-C₆ haloalkyl. In some embodiments of a compound of Formula (I), each R² is deuterium.

In some embodiments of a compound of Formula (I), n is 0. In some embodiments of a compound of Formula (I), n is 1. In some embodiments of a compound of Formula (I), n is 2. In some embodiments of a compound of Formula (I), n is 3. In some embodiments of a compound of Formula (I), n is 4. In some embodiments of a compound of Formula (I), n is 5. In some embodiments of a compound of Formula (I), n is 6. In some embodiments of a compound of Formula (I), n is 1-4. In some embodiments of a compound of Formula (I), n is 1 or 2. In some embodiments of a compound of Formula (I), n is 2-4.

Diclosed herein is a compound of Formula (II), or a pharmaceutically acceptable salt, solvate, or stereoisomer thereof:

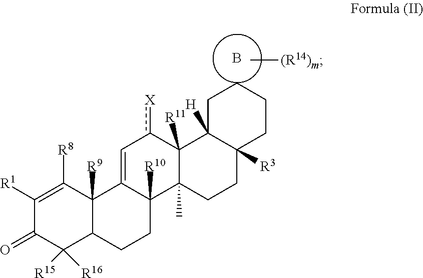

wherein:
Ring B is a cycloalkyl or a heterocycloalkyl;
each R¹⁴ is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
m is 0-6;
R¹ is hydrogen, deuterium, halogen, —CN, —OR^{b}, —NR^{c}R^{d}, —NR^{b}S(═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R³ is halogen, —CN, —OR⁵, —SR⁵, —S(═O)R⁴, —S(═O)₂NR⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁵, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3a};
each R^{3a} is independently oxo, deuterium, halogen, —CN, —OR⁵, —SR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3b};
each R^{3b} is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{4a};
each R^{4a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{d}, —C(═O)OR^{b}, —C(═O)NR^{c}R^{d}, —NR^{b}C(═NR⁸)R^{b}, —NR^{b}C(═NR^{x})NR^{c}R^{d}, —S(═O)(═NR⁸)R^{b}, —S(═O)(═NR⁸)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{d}, —C(═O)OR^{b}, —C(═O)NR^{c}R^{d}, C₁-C₆ alkyl, C₂-C₆ alkenyl, C₂-C₆ alkynyl, C_{I}—C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;

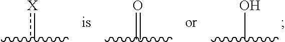

R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R¹¹ are taken together to form an optionally substituted cycloalkyl or an optionally substituted heterocycloalkyl;
R¹⁵ and R¹⁶ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹⁵ and R¹⁶ are taken together to form a cycloalkyl or a heterocycloalkyl; each substituted with 0-6 R²;
each R² is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

In some embodiments of a compound of Formula (II), R¹ is —CN, C₁-C₆ alkyl, C₁-C₆ haloalkyl, or C₁-C₆ deuteroalkyl. In some embodiments of a compound of Formula (II), R¹ is —CN or C₁-C₆ haloalkyl.

In some embodiments of a compound of Formula (II), R¹ is C₁-C₆ haloalkyl. In some embodiments of a compound of Formula (II), R¹ is —CN.

In some embodiments of a compound of Formula (II), R⁸ is hydrogen or deuterium. In some embodiments of a compound of Formula (II), R⁸ is hydrogen.

In some embodiments of a compound of Formula (II), R⁹ is C₁-C₆ alkyl or C₂-C₆ alkynyl. In some embodiments of a compound of Formula (II), R⁹ is C₁-C₆ alkyl.

In some embodiments of a compound of Formula (II), R¹⁰ is C₁-C₆ alkyl or C₂-C₆ alkynyl. In some embodiments of a compound of Formula (II), R¹⁰ is C₁-C₆ alkyl.

In some embodiments of a compound of Formula (II), R⁹ is C₁-C₆ alkyl and R¹⁰ is C₂-C₆ alkynyl.

In some embodiments of a compound of Formula (II), R¹⁰ is C₁-C₆ alkyl and R⁹ is C₂-C₆ alkynyl.

In some embodiments of a compound of Formula (II),

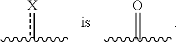

In some embodiments of a compound of Formula (II),

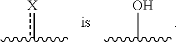

In some embodiments of a compound of Formula (II), R¹¹ is hydrogen, halogen, or —OH. In some embodiments of a compound of Formula (II), R¹¹ is hydrogen or —OH. In some embodiments of a compound of Formula (II), R¹¹ is hydrogen.

In some embodiments of a compound of Formula (II), R¹⁵ and R¹⁶ are independently hydrogen, C₁-C₆ alkyl, or C₁-C₆ deuteroalkyl. In some embodiments of a compound of Formula (II), R¹⁵ and R¹⁶ are independently hydrogen or C₁-C₆ alkyl. In some embodiments of a compound of Formula (II), R¹⁵ and R¹⁶ are independently hydrogen or C₁-C₆ deuteroalkyl. In some embodiments of a compound of Formula (II), R¹⁵ and R¹⁶ are independently C₁-C₆ alkyl.

In some embodiments of a compound of Formula (II), R¹⁵ and R¹⁶ are taken together to form a cycloalkyl. In some embodiments of a compound of Formula (II), R¹⁵ and R¹⁶ are taken together to form a cycloalkyl substituted with 1-4 deuterium. In some embodiments of a compound of Formula (II), R¹⁵ and R¹⁶ are taken together to form a cycloalkyl substituted with 1 or 2 deuterium. In some embodiments of a compound of Formula (II), R¹⁵ and R¹⁶ are taken together to form a cycloalkyl substituted with 2-4 deuteriums.

In some embodiments of a compound of Formula (II), R¹⁵ and R¹⁶ are taken together to form a heterocycloalkyl. In some embodiments of a compound of Formula (II), R¹⁵ and R¹⁶ are taken together to form a heterocycloalkyl substituted with 1-4 deuterium. In some embodiments of a compound of Formula (II), R¹⁵ and R¹⁶ are taken together to form a heterocycloalkyl substituted with 1 or 2 deuterium. In some embodiments of a compound of Formula (II), R¹⁵ and R¹⁶ are taken together to form a heterocycloalkyl substituted with 2-4 deuteriums.

In some embodiments of a compound of Formula (II), R³ is halogen, —CN, —NR⁶R⁷, —C(═O)R⁴, —OC(═O)R⁴, —C(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂R⁴, —NR⁵C(═O)R⁴, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₁-C₆ hydroxyalkyl, C₁-C₆ heteroalkyl, or heteroaryl; wherein the alkyl, alkenyl, and heteroaryl are independently optionally substituted with one, two, or three R^{3a}. In some embodiments of a compound of Formula (II), R³ is —NR⁵C(═NR^{x})R⁵ or —NR⁵C(═NR^{x})NR⁶R⁷. In some embodiments of a compound of Formula (II), R³ is —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵.

In some embodiments of a compound of Formula (II), R³ is C₁-C₆ alkyl optionally substituted with one, two, or three R^{3a}. In some embodiments of a compound of Formula (II), R³ is C₁-C₆ alkyl.

In some embodiments of a compound of Formula (II), R³ is —C(═O)OR⁵.

In some embodiments of a compound of Formula (II), each R^{3a} is independently deuterium, halogen, —CN, —OR⁵, —S(═O)R⁴, —S(═O)₂R⁴, —OC(═O)R⁴, —C(═O)OR⁵, —OC(═O)OR⁵, —C(═O)NR⁶R⁷, —OC(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, cycloalkyl, or heterocycloalkyl.

In some embodiments of a compound of Formula (II), each R^{3a} is independently deuterium, halogen, —CN, —OR⁵, —S(═O)R⁴, —S(═O)₂R⁴, —OC(═O)R⁴, —C(═O)OR⁵, —OC(═O)OR⁵, —C(═O)NR⁶R⁷, —OC(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵. In some embodiments of a compound of Formula (II), each R^{3a} is independently deuterium, halogen, —CN, —OC(═O)R⁴, —C(═O)OR⁵, —C(═O)NR⁶R⁷, —B(OR⁵)₂, —S(═O)(═NR^{x})R⁵, C₁-C₆ heteroalkyl, heterocycloalkyl, or heteroaryl. In some embodiments of a compound of Formula (II), each R^{3a} is independently —OC(═O)R⁴, —C(═O)OR⁵, —OC(═O)OR⁵, —C(═O)NR⁶R⁷, —P(═O)(R⁴)₂, —P(═OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵. In some embodiments of a compound of Formula (II), each R^{3a} is independently —OC(═O)R⁴, —C(═O)OR⁵, —OC(═O)OR⁵, or —C(═O)NR⁶R⁷. In some embodiments of a compound of Formula (II), each R^{3a} is independently —P(═O)(R⁴)₂, —P(═O)(OR⁵)₂, or —B(OR⁵)₂. In some embodiments of a compound of Formula (II), each R^{3a} is independently —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵.

In some embodiments of a compound of Formula (II), each R^{3b} is independently deuterium, halogen, —CN, —OR^{b}, C₁-C₆ alkyl, C₁-C₆ haloalkyl, or C₁-C₆ deuteroalkyl. In some embodiments of a compound of Formula (II), each R^{3b} is independently deuterium, halogen, C₁-C₆ alkyl, C₁-C₆ haloalkyl, or C₁-C₆ deuteroalkyl.

In some embodiments of a compound of Formula (II), each R⁴ is independently C₁-C₆ alkyl, C₂-C₆ alkenyl, C₂-C₆ alkynyl, C₁-C₆ haloalkyl, C₁-C₆ heteroalkyl, cycloalkyl, or heterocycloalkyl; wherein the alkyl, alkenyl, alkynyl, cycloalkyl, and heterocycloalkyl are independently optionally substituted with one, two, or three R^{4a}. In some embodiments of a compound of Formula (II), each R⁴ is independently C₁-C₆ alkyl or cycloalkyl; wherein the alkyl and cycloalkyl are independently optionally substituted with one, two, or three R^{4a}. In some embodiments of a compound of Formula (II), each R⁴ is independently C₁-C₆ alkyl optionally substituted with one, two, or three R^{4a}. In some embodiments of a compound of Formula (II), each R⁴ is independently C₁-C₆ alkyl. In some embodiments of a compound of Formula (II), each R⁴ is independently C₁-C₆ alkyl, C₁-C₆ haloalkyl, or C₁-C₆ deuteroalkyl.

In some embodiments of a compound of Formula (II), each R^{4a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (II), each R^{4a} is independently deuterium or halogen. In some embodiments of a compound of Formula (II), each R^{4a} is independently halogen.

In some embodiments of a compound of Formula (II), each R⁵ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{5a}. In some embodiments of a compound of Formula (II), each R⁵ is independently hydrogen, C₁-C₆ alkyl, or cycloalkyl; wherein the alkyl and cycloalkyl are independently optionally substituted with one, two, or three R^{5a}. In some embodiments of a compound of Formula (II), each R⁵ is independently hydrogen or C₁-C₆ alkyl optionally substituted with one, two, or three R^{5a}. In some embodiments of a compound of Formula (II), each R⁵ is independently C₁-C₆ alkyl optionally substituted with one, two, or three R^{5a}. In some embodiments of a compound of Formula (II), each R⁵ is hydrogen or C₁-C₆ alkyl. In some embodiments of a compound of Formula (II), each R⁵ is independently hydrogen, C₁-C₆ alkyl, C₁-C₆ haloalkyl, or C₁-C₆ deuteroalkyl.

In some embodiments of a compound of Formula (II), two R⁵ are taken together to form an optionally substituted heterocycloalkyl.

In some embodiments of a compound of Formula (II), each R^{5a} is independently oxo, deuterium, halogen, —CN, —OR^{b}, C₁-C₆ alkyl, C₁-C₆ haloalkyl, cycloalkyl, or heterocycloalkyl. In some embodiments of a compound of Formula (II), each R^{5a} is independently deuterium or halogen. In some embodiments of a compound of Formula (II), each R^{5a} is independently halogen. In some embodiments of a compound of Formula (II), each R^{5a} is independently —NR^{b}C(═NR^{x})R^{b} or —NR^{b}C(═NR^{x})NR^{c}R^{d}. In some embodiments of a compound of Formula (II), each R^{5a} is independently —S(═O)(═NR^{x})R^{b} or —S(═O)(═NR^{x})NR^{c}R^{d}.

In some embodiments of a compound of Formula (II), each R⁶ and R⁷ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{6a}. In some embodiments of a compound of Formula (II), each R⁶ and R⁷ is independently hydrogen, C₁-C₆ alkyl, C₁-C₆ haloalkyl, cycloalkyl, or heterocycloalkyl; wherein the alkyl, cycloalkyl, and heterocycloalkyl are independently optionally substituted with one, two, or three R^{6a}. In some embodiments of a compound of Formula (II), each R⁶ and R⁷ is independently hydrogen, C₁-C₆ alkyl, or C₁-C₆ haloalkyl; wherein the alkyl are independently optionally substituted with one, two, or three R^{6a}. In some embodiments of a compound of Formula (II), each R⁶ and R⁷ is independently hydrogen or C₁-C₆ alkyl. In some embodiments of a compound of Formula (II), each R⁶ and R⁷ is independently hydrogen, C₁-C₆ alkyl, C₁-C₆ haloalkyl, or C₁-C₆ deuteroalkyl.

In some embodiments of a compound of Formula (II), each R^{6a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (II), each R^{6a} is independently deuterium or halogen. In some embodiments of a compound of Formula (II), each R^{6a} is independently halogen. In some embodiments of a compound of Formula (II), each R^{5a} is independently —NR^{b}C(═NR^{x})R^{b} or —NR^{b}C(═NR^{x})NR^{c}R^{d}. In some embodiments of a compound of Formula (II), each R^{6a} is independently —S(═O)(═NR^{x})R^{b} or —S(═O)(═NR^{x})NR^{c}R^{d}.

In some embodiments of a compound of Formula (II), R⁶ and R⁷ are taken together with the nitrogen atom to which they are attached to form a heterocycloalkyl optionally substituted with one, two, or three R^{6b}.

In some embodiments of a compound of Formula (II), each R^{6b} is independently oxo, deuterium, halogen, —CN, —OR^{b}, —NR^{c}R^{d}, C₁-C₆ alkyl, C₁-C₆ haloalkyl, C₁-C₆ hydroxyalkyl, cycloalkyl, or heterocycloalkyl. In some embodiments of a compound of Formula (II), each R^{6b} is independently C₁-C₆ alkyl or C₁-C₆ haloalkyl. In some embodiments of a compound of Formula (II), each R^{6b} is independently C₁-C₆ alkyl.

In some embodiments of a compound of Formula (II), R^{x} is hydrogen, —NO₂, or —CN. In some embodiments of a compound of Formula (II), R^{x} is —NO₂ or —CN. In some embodiments of a compound of Formula (II), R^{x} is —CN.

In some embodiments of a compound of Formula (II), Ring B is a cycloalkyl. In some embodiments of a compound of Formula (II), Ring B is a heterocycloalkyl.

In some embodiments of a compound of Formula (II), each R¹⁴ is independently deuterium, halogen, —CN, —OR^{b}, C₁-C₆ alkyl, or C₁-C₆ haloalkyl. In some embodiments of a compound of Formula (II), each R¹⁴ is deuterium.

In some embodiments of a compound of Formula (II), m is 0. In some embodiments of a compound of Formula (II), m is 1. In some embodiments of a compound of Formula (II), m is 2. In some embodiments of a compound of Formula (II), m is 3. In some embodiments of a compound of Formula (II), m is 4. In some embodiments of a compound of Formula (II), m is 5. In some embodiments of a compound of Formula (II), m is 6. In some embodiments of a compound of Formula (II), m is 1-4. In some embodiments of a compound of Formula (II), m is 1 or 2. In some embodiments of a compound of Formula (II), m is 2-4.

Diclosed herein is a compound of Formula (III), or a pharmaceutically acceptable salt, solvate, or stereoisomer thereof:

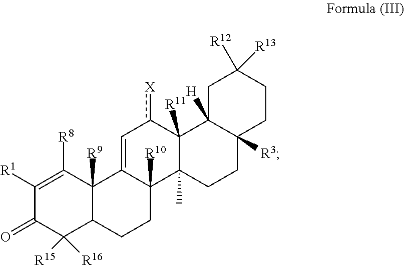

wherein:
R¹ is hydrogen, deuterium, halogen, —CN, —OR^{b}, —NR^{c}R^{d}, —NR^{b}S(═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R³ is N-linked heterocycloalkyl, a N-linked heteroaryl, —P(═O)(R⁴)₂, —P(═O)(OR⁵)₂, —B(OR⁵)₂, —S(═O)R⁴, —S(═O)₂R⁴, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, —Z—O-cycloalkyl, —Z—O-heterocycloalkyl, —Z—O-aryl, —Z—O-heteroaryl, —Z—NR⁵-cycloalkyl, —Z—NR⁵-heterocycloalkyl, —Z—NR⁵-heteroaryl, —Y(C₁-C₆alkylene)S(═O)R⁴, —Y(C₁-C₆alkylene)S(═O)₂R⁴, —Y(C₁-C₆alkylene)P(═O)(R⁴)₂, —Y(C₁-C₆alkylene)P(═O)(OR⁵)₂, —Y(C₁-C₆alkylene)B(OR⁵)₂, —Y(C₁-C₆alkylene)NR⁵C(═NR^{x})R⁵, —Y(C₁-C₆alkylene)NR⁵C(═NR^{x})NR⁶R⁷, —Y(C₁-C₆alkylene)S(═O)(═NR^{x})R⁵, —Y(C₁-C₆alkylene)S(═O)(═NR^{x})NR⁶R⁷, —Y(C₁-C₆alkylene)NR⁵S(═O)₂NR⁵C(═O)R⁵, —NR⁵C(═O)(C₁-C₆alkylene)S(═NR^{x})NR⁶R⁷, —NR⁵C(═O)(C₁-C₆alkylene)S(═NR^{x})R⁵, —Y(C₁-C₆alkylene)NR⁵S(═O)(═NR^{x})R⁵, —Y(C₂-C₆alkenylene)S(═O)₂R⁴, —Y(C₂-C₆alkenylene)P(═O)(R⁴)₂, —Y(C₂-C₆alkenylene)P(═O)(OR⁵)₂, —Y(C₂-C₆alkenylene)B(0R⁵)2, —Y(C₂-C₆alkenylene)NR⁵C(═NR^{x})R⁵, —Y(C₂-C₆alkenylene)NR⁵C(═NR^{x})NR⁶R⁷, —Y(C₂-C₆alkenylene)S(═O)(═NR^{x})R⁵, —Y(C₂-C₆alkenylene)S(═O)(═NR^{x})NR⁶R⁷, —Y(C₂-C₆alkenylene)NR⁵S(═O)₂NR⁵C(═O)R⁵, —Y(C₂-C₆alkenylene)NR⁵S(═O)(═NR^{x}R⁵, —Y(C₂-C₆alkenylene)cycloalkyl, —Y(C₂-C₆alkenylene)heterocycloalkyl, —Y(C₂-C₆alkenylene)aryl, —Y(C₂-C₆alkenylene)heteroaryl, —(C₁-C₆alkylene)OP(═O)(═O)(OR⁵)₂, —(C₁-C₆alkylene)O(C₁-C₆alkylene)OP(═O)(OR⁵)₂, or —(C₁-C₆alkylene)OP(═O)(OR⁵)[N(R⁵)₂]; wherein the alkylene, alkenylene, aryl, heteroaryl, cycloalkyl, and heterocycloalkyl are independently optionally substituted with one, two, or three R^{3a};
each R^{3a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
Y is a bond, —O—, —S—, or —NR^{b}—;
Z is a bond or C₁-C₆alkylene;
R^{x} is hydrogen, 13 NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{4a};
each R^{4a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;

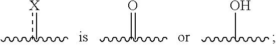

R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R¹¹ are taken together to form an optionally substituted cycloalkyl or an optionally substituted heterocycloalkyl;
R¹² and R¹³ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹² and R¹³ are taken together to form a cycloalkyl or a heterocycloalkyl; each substituted with 0-6 R¹⁴;
each R¹⁴ is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
R¹⁵ and R¹⁶ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹⁵ and R¹⁶ are taken together to form a cycloalkyl or a heterocycloalkyl; each substituted with 0-6 R²;
each R² is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

In some embodiments of a compound of Formula (III), R¹ is —CN, C₁-C₆ alkyl, C₁-C₆ haloalkyl, or C₁-C₆ deuteroalkyl. In some embodiments of a compound of Formula (III), R^{I} is —CN or C₁-C₆ haloalkyl. In some embodiments of a compound of Formula (III), W is C₁-C₆ haloalkyl. In some embodiments of a compound of Formula (III), R¹ is —CN.

In some embodiments of a compound of Formula (III), R⁸ is hydrogen or deuterium. In some embodiments of a compound of Formula (III), R⁸ is hydrogen.

In some embodiments of a compound of Formula (III), R⁹ is C₁-C₆ alkyl or C₂-C₆ alkynyl. In some embodiments of a compound of Formula (III), R⁹ is C₁-C₆ alkyl.

In some embodiments of a compound of Formula (III), R¹⁰ is C₁-C₆ alkyl or C₂-C₆ alkynyl. In some embodiments of a compound of Formula (III), R¹⁰ is C₁-C₆ alkyl.

In some embodiments of a compound of Formula (III), R⁹ is C₁-C₆ alkyl and R¹⁰ is C₂-C₆ alkynyl. In some embodiments of a compound of Formula (III), R¹⁰ is C₁-C₆ alkyl and R⁹ is C₂-C₆ alkynyl.

In some embodiments of a compound of Formula (III),

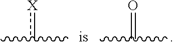

In some embodiments of a compound of Formula (III),

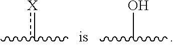

In some embodiments of a compound of Formula (III), R¹¹ is hydrogen, halogen, or —OH. In some embodiments of a compound of Formula (III), R¹¹ is hydrogen or —OH. In some embodiments of a compound of Formula (III), R¹¹ is hydrogen.

In some embodiments of a compound of Formula (III), R¹² and R¹³ are independently hydrogen, C₁-C₆ alkyl, or C₁-C₆ deuteroalkyl. In some embodiments of a compound of Formula (III), R¹² and R¹³ are independently hydrogen or C₁-C₆ alkyl. In some embodiments of a compound of Formula (III), R¹² and R¹³ are independently hydrogen or C₁-C₆ deuteroalkyl. In some embodiments of a compound of Formula (III), R¹² and R¹³ are independently C₁-C₆ alkyl.

In some embodiments of a compound of Formula (III), R¹² and R¹³ are taken together to form a cycloalkyl. In some embodiments of a compound of Formula (III), R¹² and R¹³ are taken together to form a cycloalkyl substituted with 1-4 deuterium. In some embodiments of a compound of Formula (III), R¹² and R¹³ are taken together to form a cycloalkyl substituted with 1 or 2 deuterium. In some embodiments of a compound of Formula (III), R¹² and R¹³ are taken together to form a cycloalkyl substituted with 2-4 deuteriums.

In some embodiments of a compound of Formula (III), R¹² and R¹³ are taken together to form a heterocycloalkyl. In some embodiments of a compound of Formula (III), R¹² and R¹³ are taken together to form a heterocycloalkyl substituted with 1-4 deuterium. In some embodiments of a compound of Formula (III), R¹² and R¹³ are taken together to form a heterocycloalkyl substituted with 1 or 2 deuterium. In some embodiments of a compound of Formula (III), R¹² and R¹³ are taken together to form a heterocycloalkyl substituted with 2-4 deuteriums.

In some embodiments of a compound of Formula (III), R¹⁵ and R¹⁶ are independently hydrogen, C₁-C₆ alkyl, or C₁-C₆ deuteroalkyl. In some embodiments of a compound of Formula (III), R¹⁵ and R¹⁶ are independently hydrogen or C₁-C₆ alkyl. In some embodiments of a compound of Formula (III), R¹⁵ and R¹⁶ are independently hydrogen or C₁-C₆ deuteroalkyl. In some embodiments of a compound of Formula (III), R¹⁵ and R¹⁶ are independently C₁-C₆ alkyl.

In some embodiments of a compound of Formula (III), R¹⁵ and R¹⁶ are taken together to form a cycloalkyl. In some embodiments of a compound of Formula (III), R¹⁵ and R¹⁶ are taken together to form a cycloalkyl substituted with 1-4 deuterium. In some embodiments of a compound of Formula (III), R¹⁵ and R¹⁶ are taken together to form a cycloalkyl substituted with 1 or 2 deuterium. In some embodiments of a compound of Formula (III), R¹⁵ and R¹⁶ are taken together to form a cycloalkyl substituted with 2-4 deuteriums.

In some embodiments of a compound of Formula (III), R¹⁵ and R¹⁶ are taken together to form a heterocycloalkyl. In some embodiments of a compound of Formula (III), R¹⁵ and R¹⁶ are taken together to form a heterocycloalkyl substituted with 1-4 deuterium. In some embodiments of a compound of Formula (III), R¹⁵ and R¹⁶ are taken together to form a heterocycloalkyl substituted with 1 or 2 deuterium. In some embodiments of a compound of Formula (III), R¹⁵ and R¹⁶ are taken together to form a heterocycloalkyl substituted with 2-4 deuteriums.

In some embodiments of a compound of Formula (III), R³ is —P(═O)(R⁴)₂, —P(═O)(OR⁵)₂, —Y(C₁-C₆alkylene)P(═O)(R⁴)₂, —Y(C₁-C₆alkylene)P(═O)(OR⁵)₂, —B(OR⁵)₂, —Y(C₁-C₆alkylene)B(OR⁵)₂, —S(═O)R⁴, —S(═O)₂R⁴, —Y(C₁-C₆alkylene)S(═O)R⁴, —Y(C₁-C₆alkylene)S(═O)₂R⁴, —NR⁵C(═NR^{x})R⁵, —NR⁵C(═NR^{x})NR⁶R⁷, —Y(C₁-C₆alkylene)NR⁵C(═NR^{x})R⁵, —Y(C₁-C₆alkylene)NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, —Y(C₁-C₆alkylene)S(═O)(═NR^{x})R⁵, —Y(C₁-C₆alkylene)S(═O)(═NR^{x})NR⁶R⁷, —Y(C₁-C₆alkylene)NR⁵S(═O)₂NR⁵C(═O)R⁵, —Y(C₁-C₆alkylene)NR⁵S(═O)(═NR^{x})R⁵, N-linked heterocycloalkyl, or N-linked heteroaryl. In some embodiments of a compound of Formula (III), R³ is —P(═O)(R⁴)₂, —P(═O)(OR⁵)₂, —Y(C₁-C₆alkylene)P(═O)(R⁴)₂, or —Y(C₁-C₆alkylene)P(═O)(OR⁵)₂. In some embodiments of a compound of Formula (III), R³ is —B(OR⁵)₂ or —Y(C₁-C₆alkylene)B(OR⁵)₂. In some embodiments of a compound of Formula (III), R³ is —S(═O)R⁴, —S(═O)₂R⁴, —Y(C₁-C₆alkylene)S(═O)R⁴, or —Y(C₁-C₆alkylene)S(═O)₂R⁴. In some embodiments of a compound of Formula (III), R³ is —NR⁵C(═NR^{x})R⁵, —NR⁵C(═NR^{x})NR⁶R⁷, —Y(C₁-C₆alkylene)NR⁵C(═NR^{x})R⁵, or —Y(C₁-C₆alkylene)NR⁵C(═NR^{x})NR⁶R⁷. In some embodiments of a compound of Formula (III), R³ is —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, —Y(C₁-C₆alkylene)S(═O)(═NR^{x})R⁵, —Y(C₁-C₆alkylene)S(═O)(═NR^{x})NR⁶R⁷, —Y(C₁-C₆alkylene)NR⁵S(═O)₂NR⁵C(═O)R⁵, or —Y(C₁-C₆alkylene)NR⁵S(═O)(═NR^{x})R⁵. In some embodiments of a compound of Formula (III), R³ is N-linked heterocycloalkyl or N-linked heteroaryl.

In some embodiments of a compound of Formula (III), R³ is —P(═O)(R⁴)₂, —P(═O)(OR⁵)₂, —Y(C₁-C₆alkylene)P(═O)(R⁴)₂, —Y(C₁-C₆alkylene)P(═O)(OR⁵)₂, —B(OR⁵)₂, —Y(C₁-C₆alkylene)B(OR⁵)₂, —S(═O)R⁴, —S(═O)₂R⁴, —Y(C₁-C₆alkylene)S(═O)R⁴, —Y(C₁-C₆alkylene)S(═O)₂R⁴, —NR⁵C(═NR^{x})R⁵, —NR⁵C(═NR^{x})NR⁶R⁷, —Y(C₁-C₆alkylene)NR⁵C(═NR^{x})R⁵, —Y(C₁-C₆alkylene)NR⁵C(═NR^{x})NR⁶R⁷, —NR⁵C(═O)(C₁-C₆alkylene)S(═NR^{x})NR⁶R⁷, —NR⁵C(═O)(C₁-C₆alkylene)S(═NR^{x})R⁵, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, —Y(C₁-C₆alkylene)S(═O)(═NR^{x})R⁵, —Y(C₁-C₆alkylene)S(═O)(═NR^{x})NR⁶R⁷, —Y(C₁-C₆alkylene)NR⁵S(═O)₂NR⁵C(═O)R⁵, —Y(C₁-C₆alkylene)NR⁵S(═O)(═NR^{x})R⁵, a N-linked heterocycloalkyl, or a N-linked heteroaryl.

In some embodiments of a compound of Formula (III), R³ is —Y(C₁-C₆alkylene)NR⁵C(═NR^{x})R⁵ or —Y(C₁-C₆alkylene)NR⁵C(═NR^{x})NR⁶R⁷.

In some embodiments of a compound of Formula (III), —Y is a bond. In some embodiments of a compound of Formula (III), Y is a bond or —O—.

In some embodiments of a compound of Formula (III), each R⁴ is independently C₁-C₆ alkyl, C₂-C₆ alkenyl, C₂-C₆ alkynyl, C₁-C₆ haloalkyl, C₁-C₆ heteroalkyl, cycloalkyl, or heterocycloalkyl; wherein the alkyl, alkenyl, alkynyl, cycloalkyl, and heterocycloalkyl are independently optionally substituted with one, two, or three R⁴a. In some embodiments of a compound of Formula (III), each R⁴ is independently C₁-C₆ alkyl or cycloalkyl; wherein the alkyl and cycloalkyl are independently optionally substituted with one, two, or three R^{4a}. In some embodiments of a compound of Formula (III), each R⁴ is independently C₁-C₆ alkyl optionally substituted with one, two, or three R^{4a}. In some embodiments of a compound of Formula (III), each R⁴ is independently C₁-C₆ alkyl. In some embodiments of a compound of Formula (III), each R⁴ is independently C₁-C₆ alkyl, C₁-C₆ haloalkyl, or C₁-C₆ deuteroalkyl.

In some embodiments of a compound of Formula (III), each R⁴a is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (III), each R^{4a} is independently deuterium or halogen. In some embodiments of a compound of Formula (III), each R^{4a} is independently halogen.

In some embodiments of a compound of Formula (III), each R⁵ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{5a}. In some embodiments of a compound of Formula (III), each R⁵ is independently hydrogen, C₁-C₆ alkyl, or cycloalkyl; wherein the alkyl and cycloalkyl are independently optionally substituted with one, two, or three lea. In some embodiments of a compound of Formula (III), each R⁵ is independently hydrogen or C₁-C₆ alkyl optionally substituted with one, two, or three lea. In some embodiments of a compound of Formula (III), each R⁵ is independently C₁-C₆ alkyl optionally substituted with one, two, or three R^{5a}. In some embodiments of a compound of Formula (III), each R⁵ is hydrogen or C₁-C₆ alkyl. In some embodiments of a compound of Formula (III), each R⁵ is independently hydrogen, C₁-C₆ alkyl, C₁-C₆ haloalkyl, or C₁-C₆ deuteroalkyl.

In some embodiments of a compound of Formula (III), two R⁵ are taken together to form an optionally substituted heterocycloalkyl.

In some embodiments of a compound of Formula (III), each R⁵ is independently oxo, deuterium, halogen, —CN, —OR^{b}, —NR^{c}R^{d}, —C(═O)R^{a}, —C(═O)OR^{b}, —C(═O)NR^{c}R^{d}, or C₁-C₆ alkyl. In some embodiments of a compound of Formula (III), each lea is independently halogen, —CN, —OR^{b}, —NR^{c}R^{d}, —C(═O)OR^{b}, or C₁-C₆ alkyl.

In some embodiments of a compound of Formula (III), each lea is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (III), each R^{5a} is independently deuterium or halogen. In some embodiments of a compound of Formula (III), each R^{5a} is independently halogen.

In some embodiments of a compound of Formula (III), each R⁶ and R⁷ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{6a}. In some embodiments of a compound of Formula (III), each R⁶ and R⁷ is independently hydrogen, C₁-C₆ alkyl, C₁-C₆ haloalkyl, cycloalkyl, or heterocycloalkyl; wherein the alkyl, cycloalkyl, and heterocycloalkyl are independently optionally substituted with one, two, or three R^{6a}. In some embodiments of a compound of Formula (III), each R⁶ and R—⁷ is independently hydrogen, C₁-C₆ alkyl, or C₁-C₆ haloalkyl; wherein the alkyl are independently optionally substituted with one, two, or three R^{6a}. In some embodiments of a compound of Formula (III), each R⁶ and R⁷ is independently hydrogen or C₁-C₆ alkyl. In some embodiments of a compound of Formula (III), each R⁶ and R⁷ is independently hydrogen, C₁-C₆ alkyl, C₁-C₆ haloalkyl, or C₁-C₆ deuteroalkyl.

In some embodiments of a compound of Formula (III), each R^{6a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (III), each R^{6a} is independently deuterium or halogen. In some embodiments of a compound of Formula (III), each R^{6a} is independently halogen. In some embodiments of a compound of Formula (III), each R^{5a} is independently —NR^{b}C(═NR^{x})R^{b} or —NR^{b}C(═NR^{x})NR^{c}R^{d}. In some embodiments of a compound of Formula (III), each R^{6a} is independently —S(═O)(═NR^{x})R^{b} or —S(═O)(═NR^{x})NR^{c}R^{d}.

In some embodiments of a compound of Formula (III), R⁶ and R⁷ are taken together with the nitrogen atom to which they are attached to form a heterocycloalkyl optionally substituted with one, two, or three R^{6b}.

In some embodiments of a compound of Formula (III), each R^{6b} is independently oxo, deuterium, halogen, —CN, —OR^{b}, C₁-C₆ alkyl, C₁-C₆ haloalkyl, C₁-C₆ hydroxyalkyl, cycloalkyl, or heterocycloalkyl. In some embodiments of a compound of Formula (III), each R^{6b} is independently C₁-C₆ alkyl or C₁-C₆ haloalkyl. In some embodiments of a compound of Formula (III), each R^{6b} is independently C₁-C₆ alkyl.

In some embodiments of a compound of Formula (III), R^{x} is hydrogen, —NO₂, or —CN. In some embodiments of a compound of Formula (III), R^{x} is —NO₂ or —CN. In some embodiments of a compound of Formula (III), R^{x} is —CN.

In some embodiments of a compound of Formula (III), R³ is

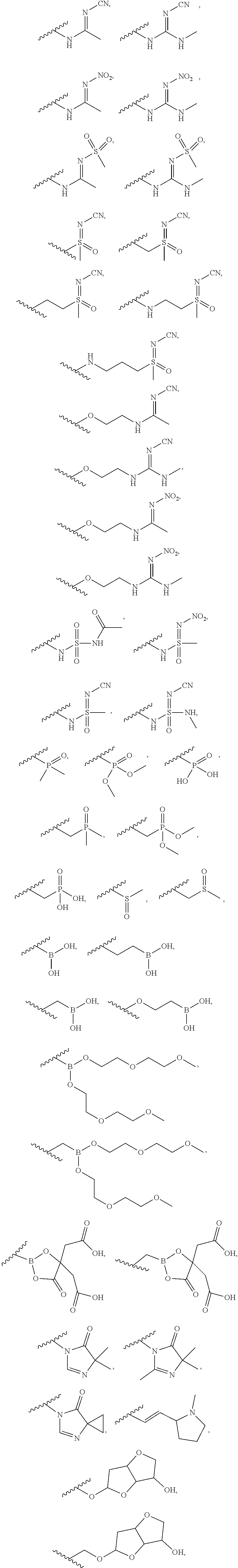

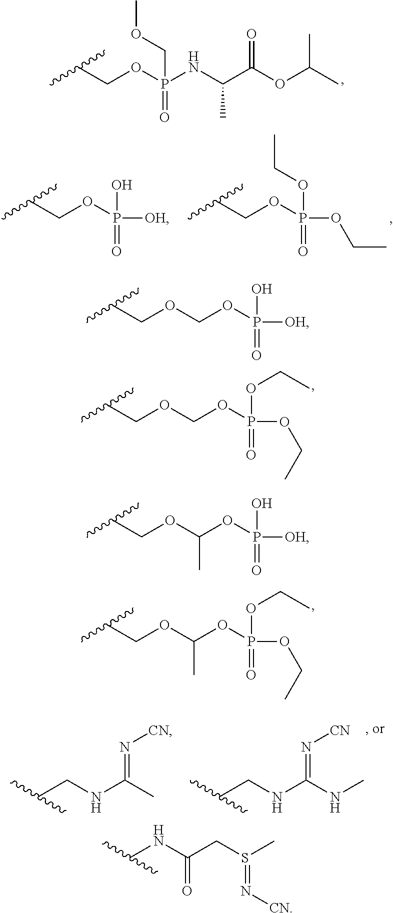

In some embodiments of a compound of Formula (III), R³ is

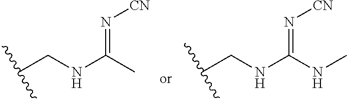

Diclosed herein is a compound of Formula (IV), or a pharmaceutically acceptable salt, solvate, or stereoisomer thereof:

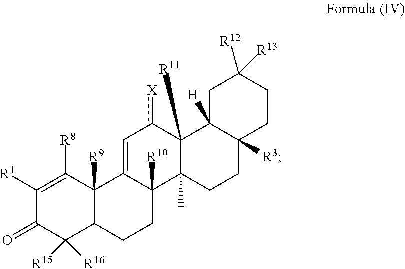

wherein:
R¹ is hydrogen, deuterium, halogen, —CN, —OR^{b}, —NR^{c}R^{d}, —NR^{b}S(═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R³ is halogen, —CN, —OR⁵, —SR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3a};
each R^{3a} is independently oxo, deuterium, halogen, —CN, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3b};
each R^{3b} is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{4a};
each R^{4a} is independently oxo, deuterium, halogen, —CN, —OR^{b}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;

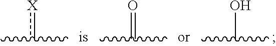

R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl; provided that one of R⁹ or R¹⁰ is C₂-C₆ alkynyl;
R¹¹ is hydrogen, deuterium, halogen, or —OR^{b};
or R³ and R¹¹ are taken together to form an optionally substituted cycloalkyl or an optionally substituted heterocycloalkyl;
R¹² and R¹³ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹² and R¹³ are taken together to form a cycloalkyl or a heterocycloalkyl; each substituted with 0-6 R¹⁴;
each R¹⁴ is independently deuterium, halogen, —CN, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
R¹⁵ and R¹⁶ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, or C₁-C₆ hydroxyalkyl;
or R¹⁵ and R¹⁶ are taken together to form a cycloalkyl or a heterocycloalkyl; each substituted with 0-6 R²;
each R² is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

In some embodiments of a compound of Formula (IV), R¹ is —CN, C₁-C₆ alkyl, C₁-C₆ haloalkyl, or C₁-C₆ deuteroalkyl. In some embodiments of a compound of Formula (IV), R¹ is —CN or C₁-C₆ haloalkyl. In some embodiments of a compound of Formula (IV), R¹ is C₁-C₆ haloalkyl. In some embodiments of a compound of Formula (IV), R¹ is —CN.

In some embodiments of a compound of Formula (IV), R⁸ is hydrogen or deuterium. In some embodiments of a compound of Formula (IV), R⁸ is hydrogen.

In some embodiments of a compound of Formula (IV), R⁹ is C₁-C₆ alkyl or C₂-C₆ alkynyl. In some embodiments of a compound of Formula (IV), R⁹ is C₂-C₆ alkynyl. In some embodiments of a compound of Formula (IV), R⁹ is C₁-C₆ alkyl.

In some embodiments of a compound of Formula (IV), R¹⁰ is C₁-C₆ alkyl or C₂-C₆ alkynyl. In some embodiments of a compound of Formula (IV), R¹⁰ is C₂-C₆ alkynyl. In some embodiments of a compound of Formula (IV), R¹⁰ is C₁-C₆ alkyl.

In some embodiments of a compound of Formula (IV), R⁹ is C₁-C₆ alkyl and R¹⁰ is C₂-C₆ alkynyl. In some embodiments of a compound of Formula (IV), R¹⁰ is C₁-C₆ alkyl and R⁹ is C₂-C₆ alkynyl.

In some embodiments of a compound of Formula (IV),

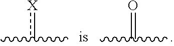

In some embodiments of a compound of Formula (IV),

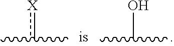

In some embodiments of a compound of Formula (IV), R¹¹ hydrogen, halogen, or —OH. In some embodiments of a compound of Formula (IV), R¹¹ is hydrogen or —OH. In some embodiments of a compound of Formula (IV), R¹¹ is hydrogen.

In some embodiments of a compound of Formula (IV), R¹² and R¹³ are independently hydrogen, C₁-C₆ alkyl, or C₁-C₆ deuteroalkyl. In some embodiments of a compound of Formula (IV), R¹² and R¹³ are independently hydrogen or C₁-C₆ alkyl. In some embodiments of a compound of Formula (IV), R¹² and R¹³ are independently hydrogen or C₁-C₆ deuteroalkyl. In some embodiments of a compound of Formula (IV), R¹² and R¹³ are independently C₁-C₆ alkyl.

In some embodiments of a compound of Formula (IV), R¹² and R¹³ are taken together to form a cycloalkyl. In some embodiments of a compound of Formula (IV), R¹² and R¹³ are taken together to form a cycloalkyl substituted with 1-4 deuterium. In some embodiments of a compound of Formula (N), R¹² and R¹³ are taken together to form a cycloalkyl substituted with 1 or 2 deuterium. In some embodiments of a compound of Formula (IV), R¹² and R¹³ are taken together to form a cycloalkyl substituted with 2-4 deuteriums.

In some embodiments of a compound of Formula (N), R¹² and R¹³ are taken together to form a heterocycloalkyl. In some embodiments of a compound of Formula (IV), R¹² and R¹³ are taken together to form a heterocycloalkyl substituted with 1-4 deuterium. In some embodiments of a compound of Formula (IV), R¹² and R¹³ are taken together to form a heterocycloalkyl substituted with 1 or 2 deuterium. In some embodiments of a compound of Formula (N), R¹² and R¹³ are taken together to form a heterocycloalkyl substituted with 2-4 deuteriums.

In some embodiments of a compound of Formula (N), R¹⁵ and R¹⁶ are independently hydrogen, C₁-C₆ alkyl, or C₁-C₆ deuteroalkyl. In some embodiments of a compound of Formula (IV), R¹⁵ and R¹⁶ are independently hydrogen or C₁-C₆ alkyl. In some embodiments of a compound of Formula (IV), R¹⁵ and R¹⁶ are independently hydrogen or C₁-C₆ deuteroalkyl. In some embodiments of a compound of Formula (IV), R¹⁵ and R¹⁶ are independently C₁-C₆ alkyl.

In some embodiments of a compound of Formula (IV), R¹⁵ and R¹⁶ are taken together to form a cycloalkyl. In some embodiments of a compound of Formula (IV), R¹⁵ and R¹⁶ are taken together to form a cycloalkyl substituted with 1-4 deuterium. In some embodiments of a compound of Formula (N), R¹⁵ and R¹⁶ are taken together to form a cycloalkyl substituted with 1 or 2 deuterium. In some embodiments of a compound of Formula (IV), R¹⁵ and R¹⁶ are taken together to form a cycloalkyl substituted with 2-4 deuteriums.

In some embodiments of a compound of Formula (IV), R¹⁵ and R¹⁶ are taken together to form a heterocycloalkyl. In some embodiments of a compound of Formula (IV), R¹⁵ and R¹⁶ are taken together to form a heterocycloalkyl substituted with 1-4 deuterium. In some embodiments of a compound of Formula (IV), R¹⁵ and R¹⁶ are taken together to form a heterocycloalkyl substituted with 1 or 2 deuterium. In some embodiments of a compound of Formula (N), R¹⁵ and R¹⁶ are taken together to form a heterocycloalkyl substituted with 2-4 deuteriums.

In some embodiments of a compound of Formula (N), R³ is halogen, —CN, —OR⁵, —NR⁶R⁷, —C(═O)R⁴, —OC(═O)R⁴, —C(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂R⁴, —NR⁵C(═O)R⁴, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₁-C₆ hydroxyalkyl, C₁-C₆ heteroalkyl, or heteroaryl; wherein the alkyl, alkenyl, and heteroaryl are independently optionally substituted with one, two, or three R^{3a}. In some embodiments of a compound of Formula (N), R³ is —NR⁵C(═NR^{x})R⁵ or —NR⁵C(═NR^{x})NR⁶R⁷. In some embodiments of a compound of Formula (N), R³ is —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵.

In some embodiments of a compound of Formula (IV), R³ is C₁-C₆ alkyl optionally substituted with one, two, or three R^{3a}. In some embodiments of a compound of Formula (N), R³ is C₁-C₆ alkyl.

In some embodiments of a compound of Formula (IV), R³ is —C(═O)O⁵.

In some embodiments of a compound of Formula (IV), each R^{3a} is independently deuterium, halogen, —CN, —OR⁵, —S(═O)R⁴, —S(═O)₂R⁴, —OC(═O)R⁴, —C(═O)OR⁵, —OC(═O)OR⁵, —C(═O)NR⁶R⁷, —OC(═O)NR⁶R⁷, —P(═O)(R₄)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, cycloalkyl, or heterocycloalkyl.

In some embodiments of a compound of Formula (IV), each R^{3a} is independently deuterium, halogen, —CN, —S(═O)R⁴, —S(═O)₂R⁴, —OC(═O)R⁴, —C(═O)OR⁵, —OC(═O)OR⁵, —C(═O)NR⁶R⁷, —OC(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵. In some embodiments of a compound of Formula (IV), each R^{3a} is independently deuterium, halogen, —CN, —OC(═O)R⁴, —C(═O)OR⁵, —C(═O)NR⁶R⁷, —B(OR⁵)₂, —S(═O)(═NR^{x})R⁵, C₁-C₆ heteroalkyl, heterocycloalkyl, or heteroaryl. In some embodiments of a compound of Formula (IV), each R^{3a} is independently —OC(═O)R⁴, —C(═O)OR⁵, —OC(═O)OR⁵, —C(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵. In some embodiments of a compound of Formula (IV), each R^{3a} is independently —OC(═O)R⁴, —C(═O)OR⁵, —OC(═O)OR⁵, or —C(═O)NR⁶R⁷. In some embodiments of a compound of Formula (IV), each R^{3a} is independently —P(═O)(R⁴)₂, —P(═O)(OR⁵)₂, or —B(OR⁵)₂. In some embodiments of a compound of Formula (IV), each R^{3a} is independently —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵.

In some embodiments of a compound of Formula (IV), each R^{3b} is independently deuterium, halogen, —CN, —OR^{b}, C₁-C₆ alkyl, C₁-C₆ haloalkyl, or C₁-C₆ deuteroalkyl. In some embodiments of a compound of Formula (IV), each R^{3b} is independently deuterium, halogen, C₁-C₆ alkyl, C₁-C₆ haloalkyl, or C₁-C₆ deuteroalkyl.

In some embodiments of a compound of Formula (IV), each R⁴ is independently C₁-C₆ alkyl, C₂-C₆ alkenyl, C₂-C₆ alkynyl, C₁-C₆ haloalkyl, C₁-C₆ heteroalkyl, cycloalkyl, or heterocycloalkyl; wherein the alkyl, alkenyl, alkynyl, cycloalkyl, and heterocycloalkyl are independently optionally substituted with one, two, or three R′. In some embodiments of a compound of Formula (IV), each R⁴ is independently C₁-C₆ alkyl or cycloalkyl; wherein the alkyl and cycloalkyl are independently optionally substituted with one, two, or three R^{4a}. In some embodiments of a compound of Formula (IV), each R⁴ is independently C₁-C₆ alkyl optionally substituted with one, two, or three R^{4a}. In some embodiments of a compound of Formula (IV), each R⁴ is independently C₁-C₆ alkyl. In some embodiments of a compound of Formula (IV), each R⁴ is independently C₁-C₆ alkyl, C₁-C₆ haloalkyl, or C₁-C₆ deuteroalkyl.

In some embodiments of a compound of Formula (IV), each R^{4a} is independently oxo, deuterium, halogen, —CN, —OR^{b}, C₁-C₆ alkyl, C₁-C₆ haloalkyl, cycloalkyl, or heterocycloalkyl. In some embodiments of a compound of Formula (IV), each R^{4a} is independently deuterium or halogen. In some embodiments of a compound of Formula (IV), each R^{4a} is independently halogen.

In some embodiments of a compound of Formula (IV), each R⁵ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{5a}. In some embodiments of a compound of Formula (IV), each R⁵ is independently hydrogen, C₁-C₆ alkyl, or cycloalkyl; wherein the alkyl and cycloalkyl are independently optionally substituted with one, two, or three R^{5a}. In some embodiments of a compound of Formula (N), each R⁵ is independently hydrogen or C₁-C₆ alkyl optionally substituted with one, two, or three R. In some embodiments of a compound of Formula (N), each R⁵ is independently C₁-C₆ alkyl optionally substituted with one, two, or three R^{5a}. In some embodiments of a compound of Formula (IV), each R⁵ is hydrogen or C₁-C₆ alkyl. In some embodiments of a compound of Formula (N), each R⁵ is independently hydrogen, C₁-C₆ alkyl, C₁-C₆ haloalkyl, or C₁-C₆ deuteroalkyl.

In some embodiments of a compound of Formula (IV), two R⁵ are taken together to form an optionally substituted heterocycloalkyl.

In some embodiments of a compound of Formula (N), each R^{5a} is independently oxo, deuterium, halogen, —CN, —OR^{b}, C₁-C₆ alkyl, C₁-C₆ haloalkyl, cycloalkyl, or heterocycloalkyl. In some embodiments of a compound of Formula (IV), each R^{5a} is independently deuterium or halogen. In some embodiments of a compound of Formula (IV), each R^{5a} is independently halogen. In some embodiments of a compound of Formula (N), each R^{5a} is independently —NR^{b}C(═NR^{x})R^{b} or —NR^{b}C(═NR^{x})NR^{c}R^{d}. In some embodiments of a compound of Formula (IV), each R^{5a} is independently —S(═O)(═NR^{x})R^{b} or —S(═O)(═NR^{x})NR^{c}R^{d}.

In some embodiments of a compound of Formula (IV), each R⁶ and R⁷ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{6a}. In some embodiments of a compound of Formula (N), each R⁶ and R⁷ is independently hydrogen, C₁-C₆ alkyl, C₁-C₆ haloalkyl, cycloalkyl, or heterocycloalkyl; wherein the alkyl, cycloalkyl, and heterocycloalkyl are independently optionally substituted with one, two, or three R^{6a}. In some embodiments of a compound of Formula (N), each R⁶ and R⁷ is independently hydrogen, C₁-C₆ alkyl, or C₁-C₆ haloalkyl; wherein the alkyl are independently optionally substituted with one, two, or three R^{6a}. In some embodiments of a compound of Formula (IV), each R⁶ and R⁷ is independently hydrogen or C₁-C₆ alkyl. In some embodiments of a compound of Formula (N), each R⁶ and R⁷ is independently hydrogen, C₁-C₆ alkyl, C₁-C₆ haloalkyl, or C₁-C₆ deuteroalkyl.

In some embodiments of a compound of Formula (N), each R^{6a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (IV), each R^{6a} is independently deuterium or halogen. In some embodiments of a compound of Formula (IV), each R^{6a} is independently halogen. In some embodiments of a compound of Formula (N), each R^{5a} is independently —NR^{b}C(═NR^{x})R^{b} or —NR^{b}C(═NR^{x})NR^{c}R^{d}. In some embodiments of a compound of Formula (IV), each R^{6a} is independently —S(═O)(═NR^{x})R^{b} or —S(═O)(═NR^{x})NR^{c}R^{d}.

In some embodiments of a compound of Formula (N), R⁶ and R⁷ are taken together with the nitrogen atom to which they are attached to form a heterocycloalkyl optionally substituted with one, two, or three R^{6b}.

In some embodiments of a compound of Formula (IV), each R^{6b} is independently oxo, deuterium, halogen, —CN, —OR^{b}, —NR^{c}R^{d}, C₁-C₆ alkyl, C₁-C₆ haloalkyl, C₁-C₆ hydroxyalkyl, cycloalkyl, or heterocycloalkyl. In some embodiments of a compound of Formula (IV), each R^{6b} is independently C₁-C₆ alkyl or C₁-C₆ haloalkyl. In some embodiments of a compound of Formula (IV), each R^{6b} is independently C₁-C₆ alkyl.

In some embodiments of a compound of Formula (IV), R^{x} is hydrogen, —NO₂, or —CN. In some embodiments of a compound of Formula (IV), R^{x} is —NO₂ or —CN. In some embodiments of a compound of Formula (IV), R^{x} is —CN.

Diclosed herein is a compound of Formula (V), or a pharmaceutically acceptable salt, solvate, or stereoisomer thereof:

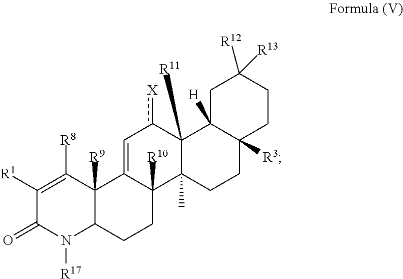

wherein:
R¹ is hydrogen, deuterium, halogen, —CN, —OR^{b}, —NR^{c}R^{d}, —NR^{b}S(═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R³ is halogen, —CN, —OR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)1V, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3a};
each R^{3a} is independently oxo, deuterium, halogen, —CN, —S(═O)R⁴, —S(═O)₂R⁴, —NO₂, —NR⁶R⁷, —S(═O)₂NR⁶R⁷, —C(═O)R⁴, —OC(═O)OR⁵, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3b};
each R^{3b} is independently deuterium, halogen, —CN, —OR^{b}, —NR^{c}R^{d}, —C(═O)R^{a}, —C(═O)OR^{b}, —C(═O)NR⁶R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{4a};
each R^{4a} is independently oxo, deuterium, halogen, —CN, —OR^{b}, —NR⁶R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR⁸)NR^{c}R^{d}, —S(═O)(═NR⁸)R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;

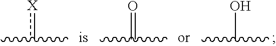

R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R¹¹ are taken together to form an optionally substituted cycloalkyl or an optionally substituted heterocycloalkyl;
R¹² and R¹³ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹² and R¹³ are taken together to form a cycloalkyl or a heterocycloalkyl; each substituted with 0-6 R¹⁴;
each R¹⁴ is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
R¹⁷ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

In some embodiments of a compound of Formula (V), R¹ is —CN, C₁-C₆ alkyl, C₁-C₆ haloalkyl, or C₁-C₆ deuteroalkyl. In some embodiments of a compound of Formula (V), R¹ is —CN or C₁-C₆ haloalkyl. In some embodiments of a compound of Formula (V), R¹ is C₁-C₆ haloalkyl. In some embodiments of a compound of Formula (V), R¹ is —CN.

In some embodiments of a compound of Formula (V), R⁸ is hydrogen or deuterium. In some embodiments of a compound of Formula (V), R⁸ is hydrogen.

In some embodiments of a compound of Formula (V), R⁹ is C₁-C₆ alkyl or C₂-C₆ alkynyl. In some embodiments of a compound of Formula (V), R⁹ is C₂-C₆ alkynyl. In some embodiments of a compound of Formula (V), R⁹ is C₁-C₆ alkyl.

In some embodiments of a compound of Formula (V), R¹⁰ is C₁-C₆ alkyl or C₂-C₆ alkynyl. In some embodiments of a compound of Formula (V), R¹⁰ is C₂-C₆ alkynyl. In some embodiments of a compound of Formula (V), R¹⁰ is C₁-C₆ alkyl.

In some embodiments of a compound of Formula (V), R⁹ is C₁-C₆ alkyl and R¹⁰ is C₂-C₆ alkynyl. In some embodiments of a compound of Formula (V), R¹⁰ is C₁-C₆ alkyl and R⁹ is C₂-C₆ alkynyl.

In some embodiments of a compound of Formula (V),

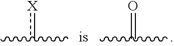

In some embodiments of a compound of Formula (V),

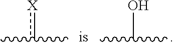

In some embodiments of a compound of Formula (V), R¹¹ is hydrogen, halogen, or —OH. In some embodiments of a compound of Formula (V), is hydrogen or —OH. In some embodiments of a compound of Formula (V), R¹¹ is hydrogen.

In some embodiments of a compound of Formula (V), R¹² and R¹³ are independently hydrogen, C₁-C₆ alkyl, or C₁-C₆ deuteroalkyl. In some embodiments of a compound of Formula (V), R¹² and R¹³ are independently hydrogen or C₁-C₆ alkyl. In some embodiments of a compound of Formula (V), R¹² and R¹³ are independently hydrogen or C₁-C₆ deuteroalkyl. In some embodiments of a compound of Formula (V), R¹² and R¹³ are independently C₁-C₆ alkyl.

In some embodiments of a compound of Formula (V), R¹³ and R¹³ are taken together to form a cycloalkyl. In some embodiments of a compound of Formula (V), R¹² and R¹³ are taken together to form a cycloalkyl substituted with 1-4 deuterium. In some embodiments of a compound of Formula (V), R¹² and R¹³ are taken together to form a cycloalkyl substituted with 1 or 2 deuterium. In some embodiments of a compound of Formula (V), R¹² and R¹³ are taken together to form a cycloalkyl substituted with 2-4 deuteriums.

In some embodiments of a compound of Formula (V), R¹² and R¹³ are taken together to form a heterocycloalkyl. In some embodiments of a compound of Formula (V), R¹² and R¹³ are taken together to form a heterocycloalkyl substituted with 1-4 deuterium. In some embodiments of a compound of Formula (V), R² and R¹³ are taken together to form a heterocycloalkyl substituted with 1 or 2 deuterium. In some embodiments of a compound of Formula (V), R¹² and R¹³ are taken together to form a heterocycloalkyl substituted with 2-4 deuteriums.

In some embodiments of a compound of Formula (V), R¹⁷ is hydrogen, C₁-C₆ alkyl, C₁-C₆ haloalkyl, or C₁-C₆ deuteroalkyl. In some embodiments of a compound of Formula (V), R¹⁷ is C₁-C₆ alkyl.

In some embodiments of a compound of Formula (V), R³ is halogen, —CN, —OR⁵, —NR⁶R⁷, —C(═O)R⁴, —OC(═O)R⁴, —C(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂R⁴, —NR⁵C(═O)R⁴, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₁-C₆ hydroxyalkyl, C₁-C₆ heteroalkyl, or heteroaryl; wherein the alkyl, alkenyl, and heteroaryl are independently optionally substituted with one, two, or three R^{3a}. In some embodiments of a compound of Formula (V), R³ is —NR⁵C(═NR^{x})R⁵ or —NR⁵C(=NR^{x})NR⁶R⁷. In some embodiments of a compound of Formula (V), R³ is —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵.

In some embodiments of a compound of Formula (V), R³ is C₁-C₆ alkyl optionally substituted with one, two, or three R^{3a}. In some embodiments of a compound of Formula (V), R³ is C₁-C₆ alkyl.

In some embodiments of a compound of Formula (V), R³ is —C(═O)OR⁵.

In some embodiments of a compound of Formula (V), each R^{3a} is independently deuterium, halogen, —CN, —OR⁵, —S(═O)R⁴, —S(═O)₂R⁴, —OC(═O)R⁴, —C(═O)OR⁵, —OC(═O)OR⁵, —C(═O)NR⁶R⁷, —OC(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, cycloalkyl, or heterocycloalkyl.

In some embodiments of a compound of Formula (V), each R^{3a} is independently deuterium, halogen, —CN, —OR⁵, —S(═O)₂R⁴, —S(═O)₂R⁴, —OC(═O)R⁴, —C(═O)OR⁵, —OC(═O)OR⁵, —C(═O)NR⁶R⁷, —OC(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵. In some embodiments of a compound of Formula (V), each R^{3a} is independently deuterium, halogen, —CN, —OR⁵, —OC(═O)R⁴, —C(═O)OR⁵, —C(═O)NR⁶R⁷, —B(OR⁵)₂, —S(═O)(═NR^{x})R⁵, C₁-C₆ heteroalkyl, heterocycloalkyl, or heteroaryl. In some embodiments of a compound of Formula (V), each R³′ is independently —OC(═O)R⁴, —C(═O)OR⁵, —OC(═O)OR⁵, —C(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵. In some embodiments of a compound of Formula (V), each R^{3a} is independently —OC(═O)R⁴, —C(═O)OR⁵, −OC(═O)OR⁵, or —C(═O)NR⁶R⁷. In some embodiments of a compound of Formula (V), each R^{3a} is independently —P(═O)(R⁴)₂, —P(═O)(OR⁵)₂, or —B(OR⁵)₂. In some embodiments of a compound of Formula (V), each R^{3a} is independently —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵.

In some embodiments of a compound of Formula (V), each R^{3b} is independently deuterium, halogen, —CN, —OR^{b}, C₁-C₆ alkyl, C₁-C₆ haloalkyl, or C₁-C₆ deuteroalkyl. In some embodiments of a compound of Formula (V), each R^{3b} is independently deuterium, halogen, C₁-C₆ alkyl, C₁-C₆ haloalkyl, or C₁-C₆ deuteroalkyl.

In some embodiments of a compound of Formula (V), each R⁴ is independently C₁-C₆ alkyl, C₂-C₆ alkenyl, C₂-C₆ alkynyl, C₁-C₆ haloalkyl, C₁-C₆ heteroalkyl, cycloalkyl, or heterocycloalkyl; wherein the alkyl, alkenyl, alkynyl, cycloalkyl, and heterocycloalkyl are independently optionally substituted with one, two, or three R^{4a}. In some embodiments of a compound of Formula (V), each R⁴ is independently C₁-C₆ alkyl or cycloalkyl; wherein the alkyl and cycloalkyl are independently optionally substituted with one, two, or three R^{4a}. In some embodiments of a compound of Formula (V), each R⁴ is independently C₁-C₆ alkyl optionally substituted with one, two, or three R^{4a}. In some embodiments of a compound of Formula (V), each R⁴ is independently C₁-C₆ alkyl. In some embodiments of a compound of Formula (V), each R⁴ is independently C₁-C₆ alkyl, C₁-C₆ haloalkyl, or C₁-C₆ deuteroalkyl.

In some embodiments of a compound of Formula (V), each R^{4a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (V), each R^{4a} is independently deuterium or halogen. In some embodiments of a compound of Formula (V), each R^{4a} is independently halogen.

In some embodiments of a compound of Formula (V), each R⁵ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{5a}. In some embodiments of a compound of Formula (V), each R⁵ is independently hydrogen, C₁-C₆ alkyl, or cycloalkyl; wherein the alkyl and cycloalkyl are independently optionally substituted with one, two, or three R^{5a}. In some embodiments of a compound of Formula (V), each R⁵ is independently hydrogen or C₁-C₆ alkyl optionally substituted with one, two, or three R^{5a}. In some embodiments of a compound of Formula (V), each R⁵ is independently C₁-C₆ alkyl optionally substituted with one, two, or three R^{5a}. In some embodiments of a compound of Formula (V), each R⁵ is hydrogen or C₁-C₆ alkyl. In some embodiments of a compound of Formula (V), each R⁵ is independently hydrogen, C₁-C₆ alkyl, C₁-C₆ haloalkyl, or C₁-C₆ deuteroalkyl.

In some embodiments of a compound of Formula (V), two R⁵ are taken together to form an optionally substituted heterocycloalkyl.

In some embodiments of a compound of Formula (V), each R^{5a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (V), each R^{5a} is independently deuterium or halogen. In some embodiments of a compound of Formula (V), each R^{5a} is independently halogen. In some embodiments of a compound of Formula (V), each R^{5a} is independently —NR^{b}C(═NR^{x})R^{b} or —NR^{b}C(═NR^{x})NR^{c}R^{d}. In some embodiments of a compound of Formula (V), each R^{5a} is independently —S(═O)(═NR^{x})R^{b} or —S(═O)(═NR^{x})NR^{c}R^{d}.

In some embodiments of a compound of Formula (V), each R⁶ and R⁷ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{6a}. In some embodiments of a compound of Formula (V), each R⁶ and R⁷ is independently hydrogen, C₁-C₆ alkyl, C₁-C₆ haloalkyl, cycloalkyl, or heterocycloalkyl; wherein the alkyl, cycloalkyl, and heterocycloalkyl are independently optionally substituted with one, two, or three R^{6a}. In some embodiments of a compound of Formula (V), each R⁶ and R⁷ is independently hydrogen, C₁-C₆ alkyl, or C₁-C₆ haloalkyl; wherein the alkyl are independently optionally substituted with one, two, or three R^{6a}. In some embodiments of a compound of Formula (V), each R⁶ and R⁷ is independently hydrogen or C₁-C₆ alkyl. In some embodiments of a compound of Formula (V), each R⁶ and R⁷ is independently hydrogen, C₁-C₆ alkyl, C₁-C₆ haloalkyl, or C₁-C₆ deuteroalkyl.

In some embodiments of a compound of Formula (V), each R^{6a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (V), each R^{h}a is independently deuterium or halogen. In some embodiments of a compound of Formula (V), each R^{6a} is independently halogen. In some embodiments of a compound of Formula (V), each R^{6a} is independently —NR^{b}C(═NR^{x})R^{b} or —NR^{b}C(═NR^{x})NR^{c}R^{d}. In some embodiments of a compound of Formula (V), each R^{6a} is independently —S(═O)(═NR^{x})R^{b} or —S(═O)(═NR^{x})NR^{c}R^{d}.

In some embodiments of a compound of Formula (V), R⁶ and R⁷ are taken together with the nitrogen atom to which they are attached to form a heterocycloalkyl optionally substituted with one, two, or three R^{6b}.

In some embodiments of a compound of Formula (V), each R^{6b} is independently oxo, deuterium, halogen, —CN, —OR^{b}, —NR^{c}R^{d}, C₁-C₆ alkyl, C₁-C₆ haloalkyl, C₁-C₆ hydroxyalkyl, cycloalkyl, or heterocycloalkyl. In some embodiments of a compound of Formula (V), each R^{6b} is independently C₁-C₆ alkyl or C₁-C₆ haloalkyl. In some embodiments of a compound of Formula (V), each R^{6b} is independently C₁-C₆ alkyl.

In some embodiments of a compound of Formula (V), R^{x} is hydrogen, —NO₂, or —CN. In some embodiments of a compound of Formula (V), R^{x} is —NO₂ or —CN. In some embodiments of a compound of Formula (V), R^{x} is —CN.

Diclosed herein is a compound of Formula (VI), or a pharmaceutically acceptable salt, solvate, or stereoisomer thereof:

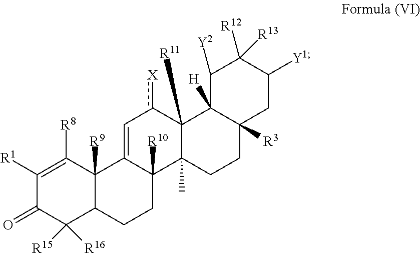

wherein:
R¹ is hydrogen, deuterium, halogen, —CN, —OR^{b}, —NR^{c}R^{d}, —NR^{b}S(═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R³ is halogen, —CN, —OR⁵, —SR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3a};
each R^{3a} is independently oxo, deuterium, halogen, —CN, —OR⁵, —SR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3b};
each R^{3b} is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{4a};
each R^{4a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(=NR^{x})R^{b}, —NR⁶C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;

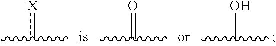

Y¹ and Y² are independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R¹¹ are taken together to form an optionally substituted cycloalkyl or an optionally substituted heterocycloalkyl;
R¹² is hydrogen, deuterium, —OR^{b}, —NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; provided that R¹² is not —CH₃;
R¹³ is —OR¹⁹, —NR²⁰R²¹, —S(═O)₂NR²⁰R²¹, —C(═O)R¹⁸, —OC(═O)R¹⁸, —C(═O)OR¹⁹, —OC(═O)OR¹⁹, —C(═O)NR²⁰R²¹, —OC(═O)NR²⁰R²¹, —NR¹⁹C(═O)NR²⁰R²¹, —NR¹⁹C(═O)OR¹⁹, —NR¹⁹S(═O)₂NR²⁰R²¹, —NR¹⁹S(═O)₂R¹⁸, —NR¹⁹C(═O)R¹⁸, C₁-C₆ alkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{13a};
each R^{13a} is independently oxo, deuterium, halogen, —CN, —SR¹⁹, —S(═O)R¹⁸, —S(═O)₂R¹⁸, —NO₂, —NR²⁰R²¹, —S(═O)₂NR²⁰R²¹, —C(═O)R¹⁸, —OC(═O)R¹⁸, —C(═O)OR¹⁹, —OC(═O)OR¹⁹, —C(═O)NR²⁰R²¹, —OC(═O)NR²⁰R²¹, —NR¹⁹C(═O)NR²⁰R²¹, —NR¹⁹C(═O)OR¹⁹, —NR¹⁹S(═O)₂NR²⁰R²¹, —NR¹⁹S(═O)₂R¹⁸, —NR¹⁹C(═O)R¹⁸, —NR¹⁹C(═O)OR¹⁹, C₁-C₆ alkyl, C₂-C₆ alkenyl, C₂-C₆ alkynyl, C₁-C₆ haloalkyl, C₁- C₆ deuteroalkyl, C₁-C₆ hydroxyalkyl, C₁-C₆ aminoalkyl, or C₁-C₆ heteroalkyl;
each R¹⁸ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{18a};
each R^{18a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C_{I}—C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R¹⁹ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{19a};
each R^{19a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C_{I}—C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R²⁰ and R²¹ are independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{20a};
each R^{20a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R²⁰ and R²¹ are taken together with the nitrogen atom to which they are attached to form a heterocycloalkyl optionally substituted with one, two, or three R^{20b};
each R^{20b} is independently oxo, deuterium, halogen, —CN, —OR^{b}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R¹⁵ and R¹⁶ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹⁵ and R¹⁶ are taken together to form a cycloalkyl or a heterocycloalkyl; each substituted with 0-6 R²;
each R² is independently deuterium, halogen, —CN, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

In some embodiments of a compound of Formula (VI), Y¹ and Y² are independently hydrogen, deuterium, halogen, —OR^{b}, C₁-C₆ alkyl, C₁-C₆ haloalkyl, or C₁-C₆ deuteroalkyl. In some embodiments of a compound of Formula (VI), Y¹ and Y² are independently hydrogen, deuterium, halogen, or —OR^{b}. In some embodiments of a compound of Formula (VI), Y¹ and Y² are independently hydrogen, deuterium, —OR^{b}, or C₁-C₆ alkyl. In some embodiments of a compound of Formula (VI), Y¹ and Y² are independently hydrogen.

In some embodiments of a compound of Formula (VI), R¹² is hydrogen, deuterium, —OR^{b}, —NR^{c}R^{d}, C₁-C₆ alkyl, C₁-C₆ haloalkyl, C₁-C₆ deuteroalkyl, C₁-C₆ hydroxyalkyl, or cycloalkyl; provided that R¹² is not —CH₃. In some embodiments of a compound of Formula (VI), R¹² is hydrogen, deuterium, C₁-C₆ alkyl, C₁-C₆ haloalkyl, or C₁-C₆ deuteroalkyl; provided that R¹² is not —CH₃. In some embodiments of a compound of Formula (VI), R¹² is hydrogen or deuterium. In some embodiments of a compound of Formula (VI), R¹² is hydrogen. In some embodiments of a compound of Formula (VI), R¹² is hydrogen or C₁-C₆ alkyl; provided that R¹² is not —CH₃. In some embodiments of a compound of Formula (VI), R¹² is C₁-C₆ alkyl; provided that R¹² is not —CH₃.

In some embodiments of a compound of Formula (VI), R¹³ _{is —NR}²⁰R²¹ , —S(═O)₂NR²⁰R²¹, —C(═O)R¹⁸, —C(═O)OR¹⁹, —C(═C)NR²⁰R²¹, —NR¹⁹C(═O )OR¹⁹, —NR¹⁹S(═O)₂NR²⁰R²¹, —NR¹⁹S(═O)₂R¹⁸, —NR¹⁹C(═O)R¹⁸, C₁-C₆ alkyl, C₁-C₆ deuteroalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{13a}. In some embodiments of a compound of Formula (VI), R¹³ is —C(═O)R¹⁸, —C(═O)OR¹⁹, —C(═O)NR²⁰R²¹, —NR¹⁹S(═O)₂NR²⁰R²¹, —NR¹⁹S(═O)₂R¹⁸, —NR¹⁹C(═O)R¹⁸, C₁-C₆ alkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{13a}. In some embodiments of a compound of Formula (VI), R¹³ is —C(═O)R¹⁸, —C(═O)OR¹⁹, —C(═O)NR²⁰R²¹, —NR¹⁹S(═O)₂R¹⁸, or —NR¹⁹C(═O)R¹⁸. In some embodiments of a compound of Formula (VI), R¹³ is —C(═O)NR²⁰R²¹, —NR¹⁹S(═O)₂NR²⁰R²¹, —NR¹⁹S(═O)₂R¹⁸, or —NR¹⁹C(═O)R¹⁸. In some embodiments of a compound of Formula (VI), R¹³ is —C(═O)NR²⁰R²¹. In some embodiments of a compound of Formula (VI), R¹³ is —NR¹⁹C(═O)R¹⁸. In some embodiments of a compound of Formula (VI), R¹³ is C₁-C₆ alkyl optionally substituted with one, two, or three R^{13a}. In some embodiments of a compound of Formula (VI), R¹³ is heteroaryl optionally substituted with one, two, or three R^{13a}.

In some embodiments of a compound of Formula (VI), each R^{13a} is independently deuterium, halogen, —CN, —OR¹⁹, —NR²⁰R²¹, —S(═O)₂NR²⁰R²¹, —C(═O)R¹⁸, —C(═O)OR¹⁹, —C(═O)NR²⁰R²¹, —NR¹⁹C(═O) NR²⁰R²¹ , —NR¹⁹C(═O)OR¹⁹, —NR¹⁹S(═O)₂NR²⁰R²¹, —NR¹⁹S(═O)₂R¹⁸, —NR¹⁹C(═O)R¹⁸, —NR¹⁹C(═O)OR¹⁹, C₁-C₆ alkyl, C₁-C₆ haloalkyl, C₁-C₆ deuteroalkyl, C₁-C₆ hydroxyalkyl, C₁-C₆ aminoalkyl, or C₁-C₆ heteroalkyl. In some embodiments of a compound of Formula (VI), each R^{13a} is independently deuterium, halogen, —CN, —OR¹⁹, —NR²⁰R²¹, C₁-C₆ alkyl, C₁-C₆ haloalkyl, C₁-C₆ deuteroalkyl, C₁-C₆ hydroxyalkyl, C₁-C₆ aminoalkyl, or C₁-C₆ heteroalkyl. In some embodiments of a compound of Formula (VI), each R^{13a} is independently deuterium, halogen, —CN, —OR¹⁹, —NR²⁰R²¹, C₁-C₆ alkyl, C₁-C₆ haloalkyl, or C₁-C₆ deuteroalkyl. In some embodiments of a compound of Formula (VI), each R^{13a} is independently —NR¹⁹S(═O)₂R¹⁸ or —NR¹⁹C(═O)R¹⁸.

Diclosed herein is a compound of Formula (VII), or a pharmaceutically acceptable salt, solvate, or stereoisomer thereof:

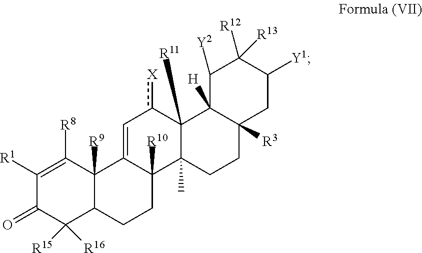

wherein:
R¹ is hydrogen, deuterium, halogen, —CN, —OR^{b}, —NR^{c}R^{d}, —NR^{b}S(═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R³ is halogen, —CN, —OR⁵, —SR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR₅C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3a};
each R^{3a} is independently oxo, deuterium, halogen, —CN, —OR⁵, —S(═O)R⁴, —S(═O)₂R⁴, —NO₂, —NR⁶R⁷, —S(═O)₂NR⁶R⁷, —C(═O)OR⁵, —OC(═O)OR⁵, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR₅C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3b};
each R^{3b} is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three lea;
each R^{4a} is independently oxo, deuterium, halogen, —CN, —OR^{b}, —NR^{c}R^{d}, —C(═O)R^{a}, —C(═O)OR^{b}, —C(═O)NR^{e}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;

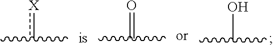

Y¹ and Y² are independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl; provided that one of Y¹ or Y² is not hydrogen;
R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R¹¹ are taken together to form an optionally substituted cycloalkyl or an optionally substituted heterocycloalkyl;
R¹² is hydrogen, deuterium, —OR^{b}, —NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R¹³ is —OR¹⁹, —NR²⁰R²¹, —S(═O)₂NR²⁰R²¹, —C(═O)R¹⁸, —OC(═O)R¹⁹, —C(═O)OR¹⁹, —OC(═O)OR¹⁹, —C(═O)NR²⁰R²¹, —OC(═O)NR²⁰R²¹, —NR¹⁹C(═)NR²⁰R²¹, —NR¹⁹C(═O)OR¹⁹, —NR¹⁹SS(═O)₂NR²⁰R²¹, —NR¹⁹S(═O)₂R¹⁸, —NR¹⁹C(═O)R¹⁸, C₁-C₆ alkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{13a};
each R^{13a} is independently oxo, deuterium, halogen, —CN, —OR″, —SR¹⁹, —S(═O)R¹⁸, —S(═O)2R¹⁸, —NO2, —NR²⁰R²¹, —S(═O)₂NR²⁰R²¹, —C(═O)R¹⁸, —OC(═O)OR¹⁹, —OC(═O)OR¹⁹, —C(═O)NR²⁰R²¹, —OC(═O)NR²⁰R²¹, —NR¹⁹C(═O)NR²⁰R²¹, —NR¹⁹C(═O)OR¹⁹, —NR¹⁹S(═O)₂NR²⁰R²¹, —NR¹⁹S(═O)₂R¹⁸, —NR¹⁹C(═O)R¹⁸, —NR¹⁹C(═O)OR¹⁹, C₁-C₆ alkyl, C₂-C₆ alkenyl, C₂-C₆ alkynyl, C₁-C₆ haloalkyl, C₁- C₆ deuteroalkyl, C₁-C₆ hydroxyalkyl, C₁-C₆ aminoalkyl, C₁-C₆ heteroalkyl;
each R¹⁸ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{18a};
each R^{18a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R¹⁹ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{19a};
each R^{19a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R²⁰ and R²¹ are independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{20a};
each R^{20a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C_{I}—C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R²⁰ and R²¹ are taken together with the nitrogen atom to which they are attached to form a heterocycloalkyl optionally substituted with one, two, or three R^{20b};
each R^{20b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C_{I}—C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R¹⁵ and R¹⁶ are independently hydrogen, —OR^{b}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
or R¹⁵ and R¹⁶ are taken together to form a cycloalkyl or a heterocycloalkyl; each substituted with 0-6 R²;
each R² is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

In some embodiments of a compound of Formula (VII), Y¹ and Y² are independently hydrogen, deuterium, halogen, —OR^{b}, C₁-C₆ alkyl, C₁-C₆ haloalkyl, or C₁-C₆ deuteroalkyl; provided that one of Y¹ or Y² is not hydrogen. In some embodiments of a compound of Formula (VII), Y¹ and Y² are independently hydrogen, deuterium, halogen, or —OR^{b}; provided that one of Y¹ or Y² is not hydrogen. In some embodiments of a compound of Formula (VII), Y¹ and Y² are independently hydrogen, deuterium, —OR^{b}, or C₁-C₆ alkyl; provided that one of Y¹ or Y² is not hydrogen.

In some embodiments of a compound of Formula (VII), R¹² is hydrogen, deuterium, —OR^{b}, —NR^{c}R^{d}, C₁-C₆ alkyl, C₁-C₆ haloalkyl, C₁-C₆ deuteroalkyl, C₁-C₆ hydroxyalkyl, or cycloalkyl. In some embodiments of a compound of Formula (VII), R¹² is hydrogen, deuterium, C₁-C₆ alkyl, C₁-C₆ haloalkyl, or C₁-C₆ deuteroalkyl. In some embodiments of a compound of Formula (VII), R¹² is hydrogen or deuterium. In some embodiments of a compound of Formula (VII), R¹² is hydrogen. In some embodiments of a compound of Formula (VII), R¹² is hydrogen or C₁-C₆ alkyl. In some embodiments of a compound of Formula (VII), R¹² is C₁-C₆ alkyl.

In some embodiments of a compound of Formula (VII), R¹³ is —NR²⁰R²¹, —S(═O)₂NR²⁰R²¹, —C(═O)R¹⁸, —C(═O)OR¹⁹, —C(═O)NR²⁰R²¹, —NR²⁰R²¹, —NR¹⁹C(═O)OR¹⁹, —NR¹⁹S(═O)₂NR²⁰R²¹, —NR¹⁹S(═O)₂R¹⁸, —NR¹⁹C(═O)R¹⁸, C₁-C₆ alkyl, C₁-C₆ deuteroalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{13a}. In some embodiments of a compound of Formula (VII), R¹³ is —C(═O)R¹⁸, —C(═O)OR¹⁹, —C(═O)NR²⁰R²¹, —NR¹⁹S(═O)₂NR²⁰R²¹, —NR¹⁹S(═O)₂R¹⁸, —NR¹⁹C(═O)R¹⁸, C₁-C₆ alkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{13a}. In some embodiments of a compound of Formula (VII), R¹³ is —C(═O)R¹⁸, —C(═O)OR¹⁹, —C(═O)NR²⁰R²¹, —NR¹⁹S(═O)₂NR²⁰R²¹, —NR¹⁹S(═O)₂R¹⁸, or —NR¹⁹C(═O)R¹⁸. In some embodiments of a compound of Formula (VII), R¹³ is —C(═O)NR²⁰R²¹, —NR¹⁹S(═O)₂NR²⁰R²¹, —NR¹⁹S(═O)₂R¹⁸, or —NR¹⁹C(═O)R¹⁸. In some embodiments of a compound of Formula (VII), R¹³ is —C(═O)NR²⁰R²¹. In some embodiments of a compound of Formula (VII), R¹³ is —NR¹⁹C(═O)R¹⁸. In some embodiments of a compound of Formula (VII), R¹³ is C₁-C₆ alkyl optionally substituted with one, two, or three R^{13a}. In some embodiments of a compound of Formula (VII), R¹³ is heteroaryl optionally substituted with one, two, or three R^{13a}.

In some embodiments of a compound of Formula (VII), each R^{13a} is independently deuterium, halogen, —CN, —OR¹⁹, —NR²⁰R²¹, —S(═O)₂NR²⁰R²¹, —C(═O)R¹⁸, —C(═O)OR¹⁹, —C(═O)NR²⁰R²¹, —NR¹⁹C(═O)NR²⁰R²¹, —NR¹⁹C(═O)OR¹⁹, —NR¹⁹S(═O)₂NR²⁰R²¹, —NR¹⁹S(═O)₂R¹⁸, —NR¹⁹C(═O)R¹⁸, —NR¹⁹C(═O)OR¹⁹, C₁-C₆ alkyl, C₁-C₆ haloalkyl, C₁-C₆ deuteroalkyl, C₁-C₆ hydroxyalkyl, C₁-C₆ aminoalkyl, or C₁-C₆ heteroalkyl. In some embodiments of a compound of Formula (VII), each R^{13a} is independently deuterium, halogen, —CN, —OR¹⁹, —NR²⁰R²¹, C₁-C₆ alkyl, C₁-C₆ haloalkyl, C₁-C₆ deuteroalkyl, C₁-C₆ hydroxyalkyl, C₁-C₆ aminoalkyl, or C₁-C₆ heteroalkyl. In some embodiments of a compound of Formula (VII), each R^{13a} is independently deuterium, halogen, —CN, —OR¹⁹, —NR²⁰R²¹, C₁-C₆ alkyl, C₁-C₆ haloalkyl, or C₁-C₆ deuteroalkyl. In some embodiments of a compound of Formula (VII), each R^{13a} is independently —NR¹⁹S(═O)₂R¹⁸ or —NR¹⁹C(═O)R¹⁸.

Diclosed herein is a compound of Formula (VIII), or a pharmaceutically acceptable salt, solvate, or stereoisomer thereof:

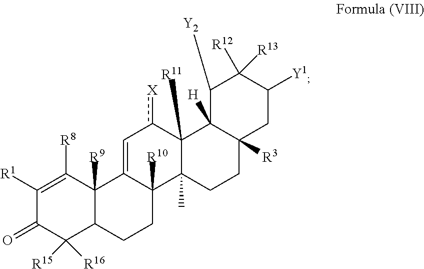

wherein:
R¹ is hydrogen, deuterium, halogen, —CN, —OR^{b}, —NR^{c}R^{d}, —NR^{b}S(═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C_{I}—C₆ haloalkyl, C₁-C₆ deuteroalkyl, C₁-C₆ hydroxyalkyl, C₁-C₆ aminoalkyl, or C₁-C₆ heteroalkyl;
R³ is halogen, —CN, —SR⁵, —S(═O)R⁴, —S(═O)₂R⁴, —NO₂, —NR⁶R⁷, —S(═O)₂NR⁶R⁷, —C(═O)R⁴, —OC(═O)R⁴, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{a})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3a};
each R^{3a} is independently oxo, deuterium, halogen, —CN, —OR⁵, —SR⁵, —S(═O)R⁴, —S(═O)₂R⁴, —NO₂, —NR⁶R⁷, —S(═O)₂NR⁶R⁷, —C(═O)R⁴, —OC(═O)OR⁵, —C(═O)OR⁵, —OC(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3b};
each R^{3b} is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{4a};
each R^{4a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(=NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;

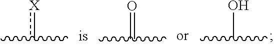

Y¹ and Y² are independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C_{I}—C₆ haloalkyl, C₁-C₆ deuteroalkyl, C₁-C₆ hydroxyalkyl, C₁-C₆ aminoalkyl, or C₁-C₆ heteroalkyl;
R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R¹¹ are taken together to form an optionally substituted cycloalkyl or an optionally substituted heterocycloalkyl;
R¹² is hydrogen, deuterium, —OR^{b}, —NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R¹³ is —CN, —OR¹⁹, —S(═O)₂NR²⁹R²¹, —OC(═O)R¹⁸, —OC(═O)OR¹⁹, —OC(═O)NR²⁰R²¹, —NR¹⁹C(═O)OR¹⁹, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, heteroaryl, —CH₂(cycloalkyl), —CH₂(heterocycloalkyl), —CH₂(aryl), or —CH₂(heteroaryl); wherein the alkyl, cycloalkyl, heterocycloalkyl, aryl, and heteroaryl are independently optionally substituted with one, two, or three R^{13a};
each R^{13a} is independently oxo, deuterium, halogen, —CN, —OR¹⁹, —SR¹⁹, —S(═O)R¹⁸, —S(═O)₂R¹⁸, —NO₂, —NR²⁰R²¹, —S(═O)₂NR²⁰R²¹, —C(═O)R¹⁸, —OC(═O)R¹⁸, —C(═O)OR¹⁹, —OC(═O)OR¹⁹, —C(═O)NR²⁰R²¹, —OC(═O)NR²⁰R²¹, —NR¹⁹C(═O)OR¹⁹, C₁-C₆ alkyl, C₂-C₆ alkenyl, C₂-C₆ alkynyl, C₁-C₆ haloalkyl, C₁- C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R¹⁸ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{18a};
each R^{18a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R¹⁹ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{19a};
each R^{19a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R²⁰ and R²¹ are independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{20a};
each R^{20a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R²⁰ and R²¹ are taken together with the nitrogen atom to which they are attached to form a heterocycloalkyl optionally substituted with one, two, or three R^{20b};
each R^{20b} is independently oxo, deuterium, halogen, —CN, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R¹⁵ and R¹⁶ are independently hydrogen, —OR^{b}, 13 NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹⁵ and R¹⁶ are taken together to form a cycloalkyl or a heterocycloalkyl; each substituted with 0-6 R²;
each R² is independently deuterium, halogen, —CN, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

In some embodiments of a compound of Formula (VIII), Y¹ and Y² are independently hydrogen, deuterium, halogen, —OR^{b}, C₁-C₆ alkyl, C₁-C₆ haloalkyl, or C₁-C₆ deuteroalkyl. In some embodiments of a compound of Formula (VIII), Y¹ and Y² are independently hydrogen, deuterium, halogen, or —OR^{b}. In some embodiments of a compound of Formula (VIII), Y¹ and Y² are independently hydrogen, deuterium, —OR^{b}, or C₁-C₆ alkyl. In some embodiments of a compound of Formula (VIII), Y¹ and Y² are hydrogen.

In some embodiments of a compound of Formula (VIII), R¹² is hydrogen, deuterium, —OR^{b}, —NR^{c}R^{d}, C₁-C₆ alkyl, C₁-C₆ haloalkyl, C₁-C₆ deuteroalkyl, C₁-C₆ hydroxyalkyl, or cycloalkyl. In some embodiments of a compound of Formula (VIII), R¹² is hydrogen, deuterium, C₁-C₆ alkyl, C₁-C₆ haloalkyl, or C₁-C₆ deuteroalkyl. In some embodiments of a compound of Formula (VIII), R¹² is hydrogen or deuterium. In some embodiments of a compound of Formula (VIII), R¹² is hydrogen. In some embodiments of a compound of Formula (VIII), R¹² is hydrogen or C₁-C₆ alkyl. In some embodiments of a compound of Formula (VIII), R¹² is C₁-C₆ alkyl.

In some embodiments of a compound of Formula (VIII), R¹³ is —CN.

In some embodiments of a compound of Formula (VIII), R¹³ is C₁-C₆ hydroxyalkyl optionally substituted with one, two, or three R^{13a}. In some embodiments of a compound of Formula (VIII), R¹³ is cycloalkyl, heterocycloalkyl, aryl, or heteroaryl; wherein the cycloalkyl, heterocycloalkyl, aryl, and heteroaryl are independently optionally substituted with one, two, or three R^{13a}. In some embodiments of a compound of Formula (VIII), R¹³ is heteroaryl optionally substituted with one, two, or three R^{13a}.

In some embodiments of a compound of Formula (VIII), R¹³ is —CN, heterocycloalkyl, heteroaryl, or —CH₂(heteroaryl); wherein the heterocycloalkyl and heteroaryl are independently optionally substituted with one, two, or three R^{13a}. In some embodiments of a compound of Formula (VIII), R¹³ is heterocycloalkyl or heteroaryl; wherein the heterocycloalkyl and heteroaryl are independently optionally substituted with one, two, or three R^{13a}. In some embodiments of a compound of Formula (VIII), R¹³ is heteroaryl optionally substituted with one, two, or three R^{13a}. In some embodiments of a compound of Formula (VIII), R¹³ is heterocycloalkyl optionally substituted with one, two, or three R^{13a}.

In some embodiments of a compound of Formula (VIII), each R^{13a} is independently deuterium, halogen, —CN, —OR¹⁹, —NR²⁰R²¹, —S(═O)₂NR²⁰R²¹, —C(═O)R¹⁸, —C(═O)OR¹⁹, —C(═O)NR²⁰R²¹, —NR¹⁹C(═O)OR¹⁹, C₁-C₆ alkyl, C₁-C₆ haloalkyl, C₁-C₆ deuteroalkyl, C₁-C₆ hydroxyalkyl, C₁-C₆ aminoalkyl, or C₁-C₆ heteroalkyl. In some embodiments of a compound of Formula (VIII), each R^{13a} is independently deuterium, halogen, —CN, —OR¹⁹, —NR²⁰R²¹, C₁-C₆ alkyl, C₁-C₆ haloalkyl, C₁-C₆ deuteroalkyl, C₁-C₆ hydroxyalkyl, C₁-C₆ aminoalkyl, or C₁-C₆ heteroalkyl. In some embodiments of a compound of Formula (VIII), each R^{13a} is independently deuterium, halogen, —CN, —OR¹⁹, —NR²⁰R²¹, C₁-C₆ alkyl, C₁-C₆ haloalkyl, or C₁-C₆ deuteroalkyl.

In some embodiments of a compound of Formula (VIII), each R^{13a} is independently oxo, —OR¹⁹, —NR²⁰R²¹, —C(═O)R¹⁸, —C(═O)OR¹⁹, —(═O)NR²⁰R²¹, C₁-C₆ alkyl, C₁-C₆ haloalkyl, C₁-C₆ deuteroalkyl, C₁-C₆ hydroxyalkyl, C₁-C₆ aminoalkyl, C₁-C₆ heteroalkyl, or aryl. In some embodiments of a compound of Formula (VIII), each R^{13a} is independently oxo, —OR¹⁹, —NR²⁰R²¹, —C(═O)NR²⁰R²¹, C₁-C₆ alkyl, C₁-C₆ haloalkyl, C₁-C₆ hydroxyalkyl, or aryl. In some embodiments of a compound of Formula (VIII), each R^{13a} is independently —OR¹⁹, —NR²⁰NR³¹, or —C(═O)NR²⁰R²¹. In some embodiments of a compound of Formula (VIII), each R^{13a} is independently —OR¹⁹ or —C(═O)NR²⁰R²¹. In some embodiments of a compound of Formula (VIII), each R^{13a} is independently —OR¹⁹.

In some embodiments of a compound of Formula (VI)-(VIII), R¹ is —CN, C₁-C₆ alkyl, C₁-C₆ haloalkyl, or C₁-C₆ deuteroalkyl. In some embodiments of a compound of Formula (VI)-(VIII), R¹ is —CN or C₁-C₆ haloalkyl. In some embodiments of a compound of Formula (VI)-(VIII), R¹ is C₁-C₆ haloalkyl. In some embodiments of a compound of Formula (VI)-(VIII), R¹ is —CN.

In some embodiments of a compound of Formula (VI)-(VIII), R⁸ is hydrogen or deuterium. In some embodiments of a compound of Formula (VI)-(VIII), R⁸ is hydrogen.

In some embodiments of a compound of Formula (VI)-(VIII), R⁹ is C₁-C₆ alkyl or C₂-C₆ alkynyl. In some embodiments of a compound of Formula (VI)-(VIII), R⁹ is C₁-C₆ alkyl.

In some embodiments of a compound of Formula (VI)-(VIII), R¹⁰ is C₁-C₆ alkyl or C₂-C₆ alkynyl. In some embodiments of a compound of Formula (VI)-(VIII), R¹⁰ is C₁-C₆ alkyl.

In some embodiments of a compound of Formula (VI)-(VIII), R⁹ is C₁-C₆ alkyl and RI° is C₂-C₆ alkynyl. In some embodiments of a compound of Formula (VI)-(VIII), R¹⁰ is C₁-C₆ alkyl and R⁹ is C₂-C₆ alkynyl.

In some embodiments of a compound of Formula (VI)-(VIII),

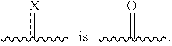

In some embodiments of a compound of Formula (VI)-(VIII),

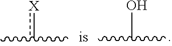

In some embodiments of a compound of Formula (VI)-(VIII), R¹¹ is hydrogen, halogen, or —OH. In some embodiments of a compound of Formula (VI)-(VIII), R¹¹ is hydrogen or —OH. In some embodiments of a compound of Formula (VI)-(VIII), R¹¹ is hydrogen.

In some embodiments of a compound of Formula (VI)-(VIII), each R¹⁸ is independently C₁-C₆ alkyl, C₂-C₆ alkenyl, C₂-C₆ alkynyl, C₁-C₆ haloalkyl, C₁-C₆ heteroalkyl, cycloalkyl, or heterocycloalkyl; wherein the alkyl, alkenyl, alkynyl, cycloalkyl, and heterocycloalkyl are independently optionally substituted with one, two, or three R^{18a}. In some embodiments of a compound of Formula (VI)-(VIII), each R¹⁸ is independently C₁-C₆ alkyl or cycloalkyl; wherein the alkyl and cycloalkyl are independently optionally substituted with one, two, or three R^{18a}. In some embodiments of a compound of Formula (VI)-(VIII), each R¹⁸ is independently C₁-C₆ alkyl optionally substituted with one, two, or three R^{18a}. In some embodiments of a compound of Formula (VI)-(VIII), each R¹⁸ is independently C₁-C₆ alkyl. In some embodiments of a compound of Formula (VI)-(VIII), each R¹⁸ is independently C₁-C₆ alkyl, C₁-C₆ haloalkyl, or C₁-C₆ deuteroalkyl.

In some embodiments of a compound of Formula (VI)-(VIII), each R^{18a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (VI)-(VIII), each R^{18a} is independently deuterium or halogen. In some embodiments of a compound of Formula (VI)-(VIII), each R^{18a} is independently halogen.

In some embodiments of a compound of Formula (VI)-(VIII), each R¹⁹ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{19a}. In some embodiments of a compound of Formula (VI)-(VIII), each R¹⁹ is independently hydrogen, C₁-C₆ alkyl, or cycloalkyl; wherein the alkyl and cycloalkyl are independently optionally substituted with one, two, or three R^{19a}. In some embodiments of a compound of Formula (VI)-(VIII), each R¹⁹ is independently hydrogen or C₁-C₆ alkyl optionally substituted with one, two, or three R^{19a}. In some embodiments of a compound of Formula (VI)-(VIII), each R¹⁹ is independently C₁-C₆ alkyl optionally substituted with one, two, or three R^{19a}. In some embodiments of a compound of Formula (VI)-(VIII), each R¹⁹ is hydrogen or C₁-C₆ alkyl. In some embodiments of a compound of Formula (VI)-(VIII), each R¹⁹ is independently hydrogen, C₁-C₆ alkyl, C₁-C₆ haloalkyl, or C₁-C₆ deuteroalkyl.

In some embodiments of a compound of Formula (VI)-(VIII), each R^{19a} is independently oxo, deuterium, halogen, —CN, —OR^{b}, C₁-C₆ alkyl, C₁-C₆ haloalkyl, cycloalkyl, or heterocycloalkyl. In some embodiments of a compound of Formula (VI)-(VIII), each R^{19a} is independently deuterium or halogen. In some embodiments of a compound of Formula (VI)-(VIII), each R^{19a} is independently halogen.

In some embodiments of a compound of Formula (VI)-(VIII), each R²⁰ and R²¹ are independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{20a}. In some embodiments of a compound of Formula (VI)-(VIII), each R²⁰ and R²¹ are independently hydrogen, C₁-C₆ alkyl, C₁-C₆ haloalkyl, cycloalkyl, or heterocycloalkyl; wherein the alkyl, cycloalkyl, and heterocycloalkyl are independently optionally substituted with one, two, or three R^{6a}. In some embodiments of a compound of Formula (VI)-(VIII), each R²⁰ and R²¹ are independently hydrogen, C₁-C₆ alkyl, or C₁-C₆ haloalkyl; wherein the alkyl are independently optionally substituted with one, two, or three R. In some embodiments of a compound of Formula (VI)-(VIII), each R²⁰ and R²¹ are independently hydrogen or C₁-C₆ alkyl. In some embodiments of a compound of Formula (VI)-(VIII), each R²⁰ and R²¹ are independently hydrogen, C₁-C₆ alkyl, C₁-C₆ haloalkyl, or C₁-C₆ deuteroalkyl.

In some embodiments of a compound of Formula (VI)-(VIII), each R^{20a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (VI)-(VIII), each R^{20a} is independently deuterium or halogen. In some embodiments of a compound of Formula (VI)-(VIII), each R^{20a} is independently halogen. In some embodiments of a compound of Formula (VI)-(VIII), each R^{20a} is independently —NR^{b}C(═NR^{x})R^{b} or —NR^{b}C(═NR^{x})NR^{c}R^{d}. In some embodiments of a compound of Formula (VI)-(VIII), each R^{20a} is independently —S(═O)(═NR^{x})R^{b} or —S(═O)(═NR^{x})NR^{c}R^{d}.

In some embodiments of a compound of Formula (VI)-(VIII), R²⁰ and R²¹ are taken together with the nitrogen atom to which they are attached to form a heterocycloalkyl optionally substituted with one, two, or three R^{20b}.

In some embodiments of a compound of Formula (VI)-(VIII), each R^{20b} is independently oxo, deuterium, halogen, —CN, —OR^{b}, —NR^{c}R^{d}, C₁-C₆ alkyl, C₁-C₆ haloalkyl, C₁-C₆ hydroxyalkyl, cycloalkyl, or heterocycloalkyl. In some embodiments of a compound of Formula (VI)-(VIII), each R^{20b} is independently C₁-C₆ alkyl or C₁-C₆ haloalkyl. In some embodiments of a compound of Formula (VI)-(VIII), each R^{20b} is independently C₁-C₆ alkyl.

In some embodiments of a compound of Formula (VI)-(VIII), R³ is halogen, —CN, —OR⁵, —NR⁶R⁷, —C(═O)R⁴, —OC(═O)R⁴, —C(═O)OR⁵, —C(═O)NR⁶R⁷, —OC(═O)NR⁶R⁷, —NR⁵C(═O)NR⁶R⁷, —NR⁵C(═O)OR⁵, —NR⁵S(═O)₂R⁴, —NR⁵C(═O)R⁴, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₁-C₆ hydroxyalkyl, C₁-C₆ heteroalkyl, or heteroaryl; wherein the alkyl, alkenyl, and heteroaryl are independently optionally substituted with one, two, or three R^{3a}. In some embodiments of a compound of Formula (VI)-(VIII), R³ is —NR⁵C(═NR^{x})R⁵ or —NR⁵C(═NR^{x})NR⁶R⁷. In some embodiments of a compound of Formula (VI)-(VIII), R³ is —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵.

In some embodiments of a compound of Formula (VI)-(VIII), R³ is C₁-C₆ alkyl or —C(═O)OR⁵. In some embodiments of a compound of Formula (VI)-(VIII), R³ is C₁-C₆ alkyl optionally substituted with one, two, or three R^{3a}. In some embodiments of a compound of Formula (VI)-(VIII), R³ is C₁-C₆ alkyl. In some embodiments of a compound of Formula (VI)-(VIII), R³ is —C(═O)OR⁵.

In some embodiments of a compound of Formula (VI)-(VIII), each R^{3a} is independently deuterium, halogen, —CN, —OR⁵, —S(═O)R⁴, —S(═O)₂R⁴, —OC(═O)R⁴, —C(═O)OR⁵, —OC(═O)OR⁵, —C(═O)NR⁶R⁷, —OC(═O)NR⁶R⁷, —P(═O)(R⁴)₂, —P(═O)(OR⁵)₂, —B(OR⁵)₂, —NR⁵C(=NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, cycloalkyl, or heterocycloalkyl. In some embodiments of a compound of Formula (VI)-(VIII), each R^{3a} is independently deuterium, halogen, —CN, —S(═O)R⁴, —S(═O)₂R⁴, —OC(═O)R⁴, —C(═O)OR⁵, —OC(═O)OR⁵, —C(═O)NR⁶R⁷, —OC(═O)NR⁶R⁷, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(=NW)R⁵, —S(═O)(=NR^{x})NR⁶R⁷, —NR⁵S(═O)₂NR⁵C(═O)R⁵, or —NR⁵S(═O)(═NR^{x})R⁵. In some embodiments of a compound of Formula (VI)-(VIII), each R^{3a} is independently deuterium, halogen, —CN, —OC(═O)R⁴, —C(═O)OR⁵, —C(═O)NR⁶R⁷, —B(OR⁵)₂, —S(═O)(═NR^{x})R⁵, C₁-C₆ heteroalkyl, heterocycloalkyl, or heteroaryl. In some embodiments of a compound of Formula (VI)-(VIII), each R^{3a} is independently —OC(═O)R⁴, —C(═O)OR⁵, —OC(═O)OR⁵, —C(═O)NR⁶R⁷, —P(═O)(R⁴)₂, —P(═O)(OR⁵)₂, —B(OR⁵)₂, —NR^{x}(═NR^{x})R⁵, —NR⁵C(═NR^{x})NR⁶R⁷, —S(═O)(═NR^{x})R⁵, —S(═O)(═NR^{x})NR⁵R⁷, —NR⁵S(═O)₂NR⁵C(═O)R⁵, or —NR⁵S(═O)(═NR^{x})R⁵. In some embodiments of a compound of Formula (VI)-(VIII), each R^{3a} is independently —OC(═O)R⁴, —C(═O)OR⁵, —OC(═O)OR⁵, or —C(═O)NR⁶R⁷. In some embodiments of a compound of Formula (VI)-(VIII), each R^{3a} is independently —P(═O)(R⁴)₂, —P(═O)(OR⁵)₂, or —B(OR⁵)₂. In some embodiments of a compound of Formula (VI)-(VIII), each R^{3a} is independently —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, or —NR⁵S(═O)(═NR^{x})R⁵.

In some embodiments of a compound of Formula (VI)-(VIII), each R^{3b} is independently deuterium, halogen, —CN, —OR^{b}, C₁-C₆ alkyl, C₁-C₆ haloalkyl, or C₁-C₆ deuteroalkyl. In some embodiments of a compound of Formula (VI)-(VIII), each R^{3b} is independently deuterium, halogen, C₁-C₆ alkyl, C₁-C₆ haloalkyl, or C₁-C₆ deuteroalkyl.

In some embodiments of a compound of Formula (VI)-(VIII), each R⁴ is independently C₁-C₆ alkyl, C₂-C₆ alkenyl, C₂-C₆ alkynyl, C₁-C₆ haloalkyl, C₁-C₆ heteroalkyl, cycloalkyl, or heterocycloalkyl; wherein the alkyl, alkenyl, alkynyl, cycloalkyl, and heterocycloalkyl are independently optionally substituted with one, two, or three R^{4a}. In some embodiments of a compound of Formula (VI)-(VIII), each R⁴ is independently C₁-C₆ alkyl or cycloalkyl; wherein the alkyl and cycloalkyl are independently optionally substituted with one, two, or three R^{4a}. In some embodiments of a compound of Formula (VI)-(VIII), each R⁴ is independently C₁-C₆ alkyl optionally substituted with one, two, or three R^{4a}. In some embodiments of a compound of Formula (VI)-(VIII), each R⁴ is independently C₁-C₆ alkyl. In some embodiments of a compound of Formula (VI)-(VIII), each R⁴ is independently C₁-C₆ alkyl, C₁-C₆ haloalkyl, or C₁-C₆ deuteroalkyl.

In some embodiments of a compound of Formula (VI)-(VIII), each R^{4a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (VI)-(VIII), each R^{4a} is independently deuterium or halogen. In some embodiments of a compound of Formula (VI)-(VIII), each R^{4a} is independently halogen.

In some embodiments of a compound of Formula (VI)-(VIII), each R⁵ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{5a}. In some embodiments of a compound of Formula (VI)-(VIII), each R⁵ is independently hydrogen, C₁-C₆ alkyl, or cycloalkyl; wherein the alkyl and cycloalkyl are independently optionally substituted with one, two, or three R^{5a}. In some embodiments of a compound of Formula (VI)-(VIII), each R⁵ is independently hydrogen or C₁-C₆ alkyl optionally substituted with one, two, or three R^{5a}. In some embodiments of a compound of Formula (VI)-(VIII), each R⁵ is independently C₁-C₆ alkyl optionally substituted with one, two, or three R^{5a}. In some embodiments of a compound of Formula (VI)-(VIII), each R⁵ is hydrogen or C₁-C₆ alkyl. In some embodiments of a compound of Formula (VI)-(VIII), each R⁵ is independently hydrogen, C₁-C₆ alkyl, C₁-C₆ haloalkyl, or C₁-C₆ deuteroalkyl.

In some embodiments of a compound of Formula (VI)-(VIII), two R⁵ are taken together to form an optionally substituted heterocycloalkyl.

In some embodiments of a compound of Formula (VI)-(VIII), each R^{5a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (VI)-(VIII), each R^{5a} is independently deuterium or halogen. In some embodiments of a compound of Formula (VI)-(VIII), each R^{5a} is independently halogen. In some embodiments of a compound of Formula (VI)-(VIII), each R^{5a} is independently —NR^{b}C(═NR^{x})R^{b} or —NR^{b}C(═NR^{x})NR^{c}R^{d}. In some embodiments of a compound of Formula (VI)-(VIII), each R^{5a} is independently —S(═O)(═NR^{x})R^{b} or —S(═O)(═NR^{x})NR^{c}R^{d}.

In some embodiments of a compound of Formula (VI)-(VIII), each R⁶ and R⁷ is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three R^{6a}. In some embodiments of a compound of Formula (VI)-(VIII), each R⁶ and R⁷ is independently hydrogen, C₁-C₆ alkyl, C₁-C₆ haloalkyl, cycloalkyl, or heterocycloalkyl; wherein the alkyl, cycloalkyl, and heterocycloalkyl are independently optionally substituted with one, two, or three R^{6a}. In some embodiments of a compound of Formula (VI)-(VIII), each R⁶ and R⁷ is independently hydrogen, C₁-C₆ alkyl, or C₁-C₆ haloalkyl; wherein the alkyl are independently optionally substituted with one, two, or three R^{6a}. In some embodiments of a compound of Formula (VI)-(VIII), each R⁶ and R⁷ is independently hydrogen or C₁-C₆ alkyl. In some embodiments of a compound of Formula (VI)-(VIII), each R⁶ and R7 is independently hydrogen, C₁-C₆ alkyl, C₁-C₆ haloalkyl, or C₁-C₆ deuteroalkyl.

In some embodiments of a compound of Formula (VI)-(VIII), each R^{6a} is independently oxo, deuterium, halogen, —CN, —OR^{b}, —NR^{c}R^{d}, C₁-C₆ alkyl, C₁-C₆ haloalkyl, cycloalkyl, or heterocycloalkyl. In some embodiments of a compound of Formula (VI)-(VIII), each R^{h}a is independently deuterium or halogen. In some embodiments of a compound of Formula (VI)-(VIII), each R^{6a} is independently halogen. In some embodiments of a compound of Formula (VI)-(VIII), each R^{5a} is independently —NR^{b}C(═NR^{x})R^{b} or —NR^{b}C(═NR^{x})NR^{c}R^{d}. In some embodiments of a compound of Formula (VI)-(VIII), each R^{6a} is independently —S(═O)(=NR^{x})R^{b} or —S(═O)(═NR^{x})NR^{c}R^{d}.

In some embodiments of a compound of Formula (VI)-(VIII), R⁶ and R⁷ are taken together with the nitrogen atom to which they are attached to form a heterocycloalkyl optionally substituted with one, two, or three R^{6b}.

In some embodiments of a compound of Formula (VI)-(VIII), each R^{6b} is independently oxo, deuterium, halogen, —CN, —OR^{b}, —NR^{c}R^{d}, C₁-C₆ alkyl, C₁-C₆ haloalkyl, C₁-C₆ hydroxyalkyl, cycloalkyl, or heterocycloalkyl. In some embodiments of a compound of Formula (VI)-(VIII), each R^{6b} is independently C₁-C₆ alkyl or C₁-C₆ haloalkyl. In some embodiments of a compound of Formula (VI)-(VIII), each R^{6b} is independently C₁-C₆ alkyl.

In some embodiments of a compound of Formula (VI)-(VIII), R^{x} is hydrogen, —NO₂, or —CN. In some embodiments of a compound of Formula (VI)-(VIII), R^{x} is —NO₂ or —CN. In some embodiments of a compound of Formula (VI)-(VIII), R^{x} is —CN.

In some embodiments of a compound of Formula (VI)-(VIII), R¹⁵ and R¹⁶ are independently hydrogen, C₁-C₆ alkyl, or C₁-C₆ deuteroalkyl. In some embodiments of a compound of Formula (VI)-(VIII), R¹⁵ and R¹⁶ are independently hydrogen or C₁-C₆ alkyl. In some embodiments of a compound of Formula (VI)-(VIII), R¹⁵ and R¹⁶ are independently hydrogen or C₁-C₆ deuteroalkyl. In some embodiments of a compound of Formula (VI)-(VIII), R¹⁵ and R¹⁶ are independently C₁-C₆ alkyl.

In some embodiments of a compound of Formula (VI)-(VIII), R¹⁵ and R¹⁶ are taken together to form a cycloalkyl. In some embodiments of a compound of Formula (VI)-(VIII), R¹⁵ and R¹⁶ are taken together to form a cycloalkyl substituted with 1-4 deuterium. In some embodiments of a compound of Formula (VI)-(VIII), R¹⁵ and R¹⁶ are taken together to form a cycloalkyl substituted with 1 or 2 deuterium. In some embodiments of a compound of Formula (VI)-(VIII), R¹⁵ and R¹⁶ are taken together to form a cycloalkyl substituted with 2-4 deuteriums.

In some embodiments of a compound of Formula (VI)-(VIII), R¹⁵ and R¹⁶ are taken together to form a heterocycloalkyl. In some embodiments of a compound of Formula (VI)-(VIII), R¹⁵ and R¹⁶ are taken together to form a heterocycloalkyl substituted with 1-4 deuterium. In some embodiments of a compound of Formula (VI)-(VIII), R¹⁵ and R¹⁶ are taken together to form a heterocycloalkyl substituted with 1 or 2 deuterium. In some embodiments of a compound of Formula (VI)-(VIII), R¹⁵ and R¹⁶ are taken together to form a heterocycloalkyl substituted with 2-4 deuteriums.

In some embodiments of a compound of Formula (I)-(VIII), each R^{a} is independently C₁-C₆ alkyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl; wherein the alkyl, alkenyl, alkynyl, cycloalkyl, and heterocycloalkyl are independently optionally substituted with one, two, or three deuterium, halogen, —OH, —NH₂, or C₁-C₆ alkyl. In some embodiments of a compound of Formula (I)-(VIII), each R^{a} is independently C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three deuterium, halogen, —OH, —NH₂, or C₁-C₆ alkyl. In some embodiments of a compound of Formula (I)-(VIII), each R^{a} is independently C₁-C₆ alkyl, C₁-C₆ haloalkyl, or C₁-C₆ deuteroalkyl; wherein the alkyl are independently optionally substituted with one, two, or three deuterium, halogen, —OH, —NH₂, or C₁-C₆ alkyl. In some embodiments of a compound of Formula (I)-(VIII), each R^{a} is independently C₁-C₆ alkyl optionally substituted with one, two, or three deuterium, halogen, —OH, —NH₂, or C₁-C₆ alkyl. In some embodiments of a compound of Formula (I)-(VIII), each R^{a} is independently C₁-C₆ alkyl.

In some embodiments of a compound of Formula (I)-(VIII), each R^{b} is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three deuterium, halogen, —OH, —NH₂, or C₁-C₆ alkyl. In some embodiments of a compound of Formula (I)-(VIII), each R^{b} is independently hydrogen, C₁-C₆ alkyl, C₁-C₆ haloalkyl, or C₁-C₆ deuteroalkyl; wherein the alkyl are independently optionally substituted with one, two, or three deuterium, halogen, —OH, —NH₂, or C₁-C₆ alkyl. In some embodiments of a compound of Formula (I)-(VIII), each R^{b} is independently hydrogen or C₁-C₆ alkyl optionally substituted with one, two, or three deuterium, halogen, —OH, —NH₂, or C₁-C₆ alkyl. In some embodiments of a compound of Formula (I)-(VIII), each R^{b} is independently hydrogen or C₁-C₆ alkyl. In some embodiments of a compound of Formula (I)-(VIII), each R^{b} is independently hydrogen. In some embodiments of a compound of Formula (I)-(VIII), each R^{b} is independently C₁-C₆ alkyl.

In some embodiments of a compound of Formula (I)-(VIII), each R^{c} and R^{d} is independently hydrogen, C₁-C₆ alkyl, C₁-C₆ haloalkyl, C₁-C₆ deuteroalkyl, cycloalkyl, or heterocycloalkyl; wherein the alkyl, cycloalkyl, and heterocycloalkyl are independently optionally substituted with one, two, or three deuterium, halogen, —OH, —NH₂, or C₁-C₆ alkyl. In some embodiments of a compound of Formula (I)-(VIII), each R^{c} and R^{d} is independently hydrogen, C₁-C₆ alkyl, C₁-C₆ haloalkyl, or C₁-C₆ deuteroalkyl; wherein the alkyl are independently optionally substituted with one, two, or three deuterium, halogen, —OH, —NH₂, or C₁-C₆ alkyl. In some embodiments of a compound of Formula (I)-(VIII), each W and R^{d} is independently hydrogen or C₁-C₆ alkyl optionally substituted with one, two, or three deuterium, halogen, —OH, —NH₂, or C₁-C₆ alkyl. In some embodiments of a compound of Formula (I)-(VIII), each R^{c} and R^{d} is independently hydrogen or C₁-C₆ alkyl. In some embodiments of a compound of Formula (I)-(VIII), each R^{b} is independently hydrogen. In some embodiments of a compound of Formula (I)-(VIII), each R^{c} and R^{d} is independently C₁-C₆ alkyl.

In some embodiments of a compound of Formula (I)-(VIII), R^{c} and R^{d} are taken together with the nitrogen atom to which they are attached to form a heterocycloalkyl optionally substituted with one, two, or three deuterium, halogen, —OH, —NH₂, or C₁-C₆ alkyl.

Disclosed herein is a compound, or a pharmaceutically acceptable salt, solvate, or stereoisomer thereof, selected from the group consisting of:

| Ex. | Structure |
|---|---|
| Con- trol | 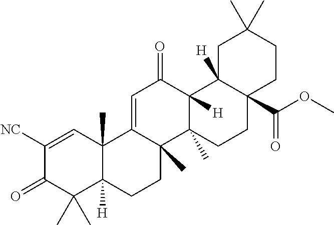 |
| 1 | 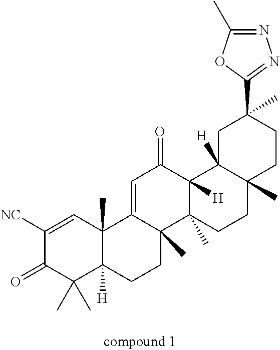 |
| 2 | 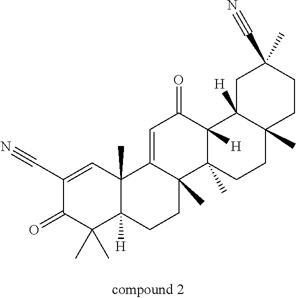 |
| 3 | 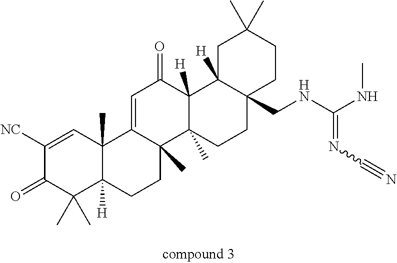 [SMILES: CNC(=NC#N)NC[C@]12CCC(C)(C)C[C@H]1[C@H]1C(=O)C=C3[C@@]4(C)C=C(C#N)C(=O)C(C)(C)[C@@H]4CC[C@@]3(C)[C@]1(C)CC2] |
| 4 | 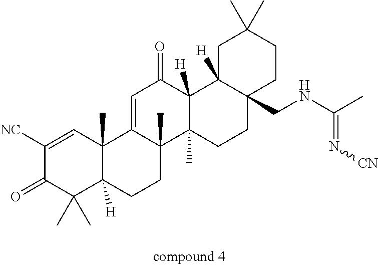 |
| 5 | 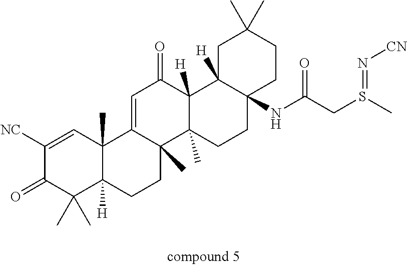 [SMILES: C/S(CC(=O)N[C@]12CCC(C)(C)C[C@H]1[C@H]1C(=O)C=C3[C@@]4(C)C=C(C#N)C(=O)C(C)(C)[C@@H]4CC[C@@]3(C)[C@]1(C)CC2)=N\C#N] |
| 6 | 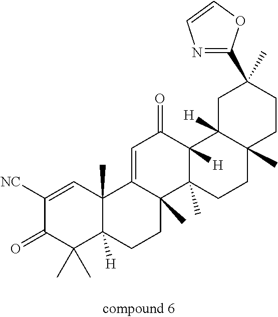 |
| 7 | 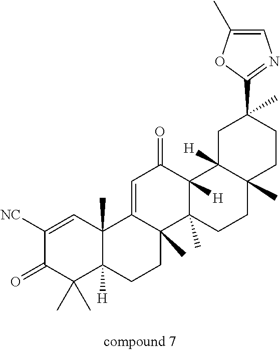 |
| 8 | 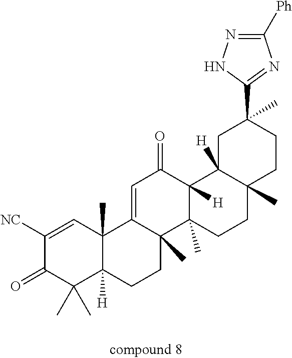 |
| 9 | 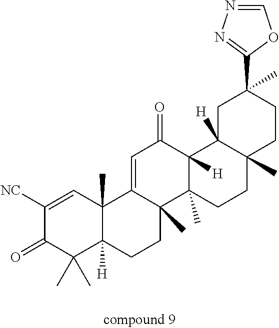 |
| 10 | 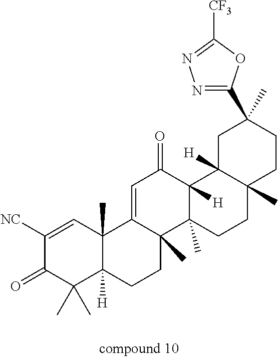 |
| 11 | 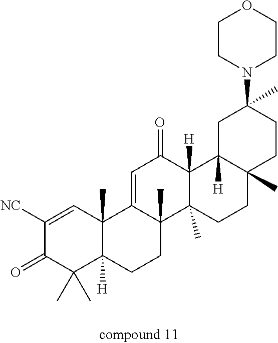 |
| 12 | 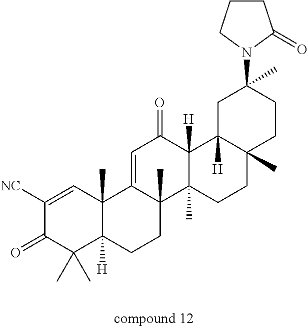 |
| 13 | 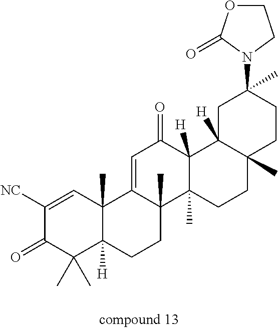 |
| 14 | 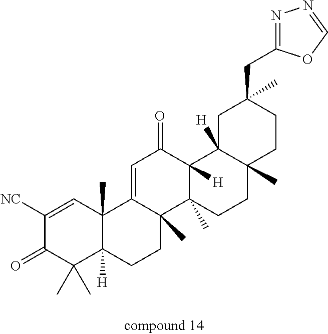 |
| 15 | 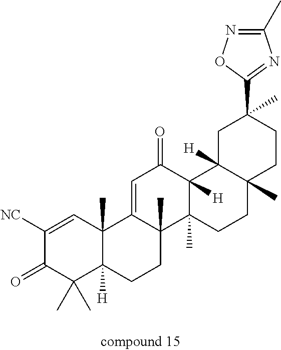 |
| 16 | 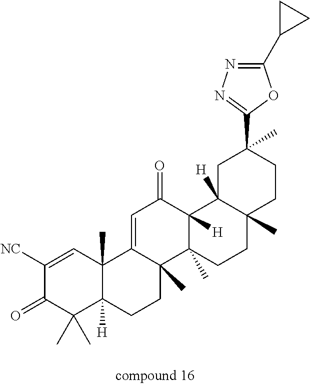 |
| 17 | 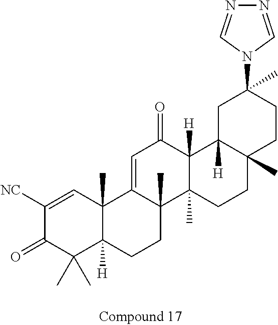 |
| 18 | 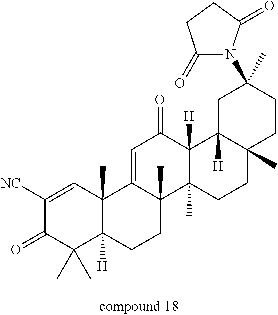 |
| 19 | 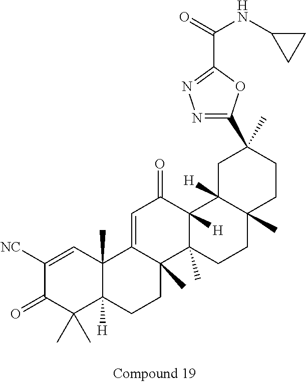 |
| 20 | 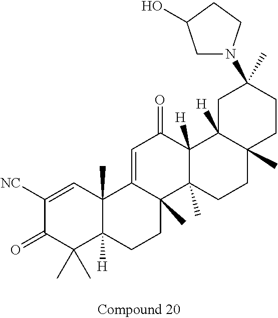 [SMILES: CC1(C)C(=O)C(C#N)=C[C@]2(C)C3=CC(=O)[C@@H]4[C@@H]5C[C@@](C)(N6CCC(O)C6)CC[C@]5(C)CC[C@@]4(C)[C@]3(C)CC[C@@H]12] |
| 21 | 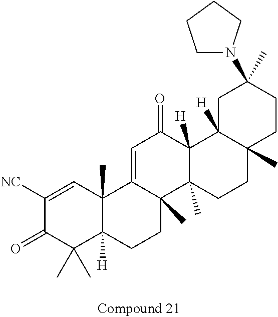 [SMILES: CC1(C)C(=O)C(C#N)=C[C@]2(C)C3=CC(=O)[C@@H]4[C@@H]5C[C@@](C)(N6CCCC6)CC[C@]5(C)CC[C@@]4(C)[C@]3(C)CC[C@@H]12] |
| 23 | 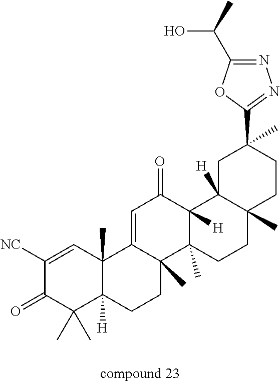 |
| 24 | 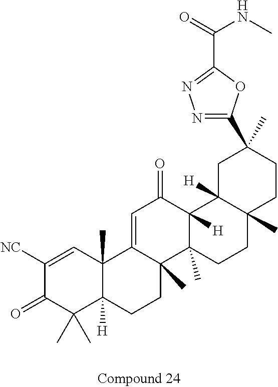 |
| 25 | 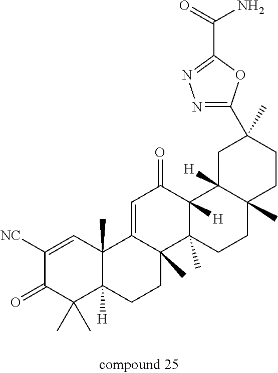 |
| 26 | 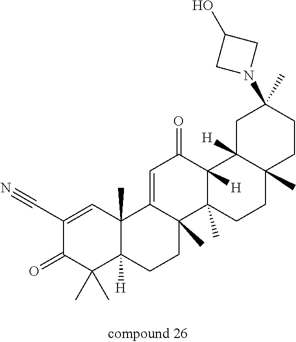 |
| 27 | 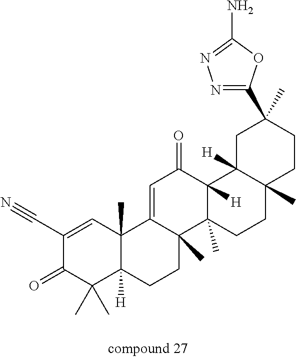 |

Disclosed herein is a compound, or a pharmaceutically acceptable salt, solvate, or stereoisomer thereof, selected from the group consisting of:

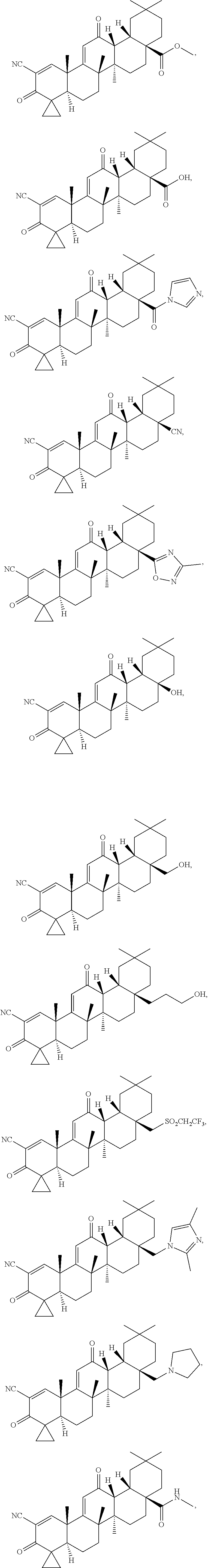

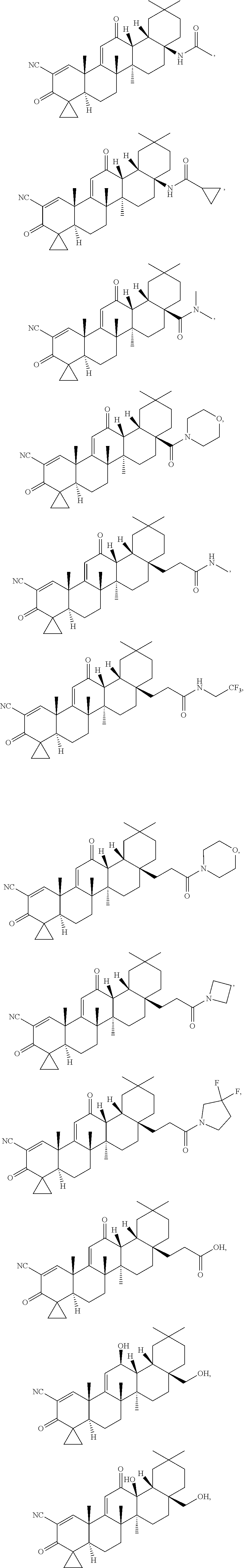

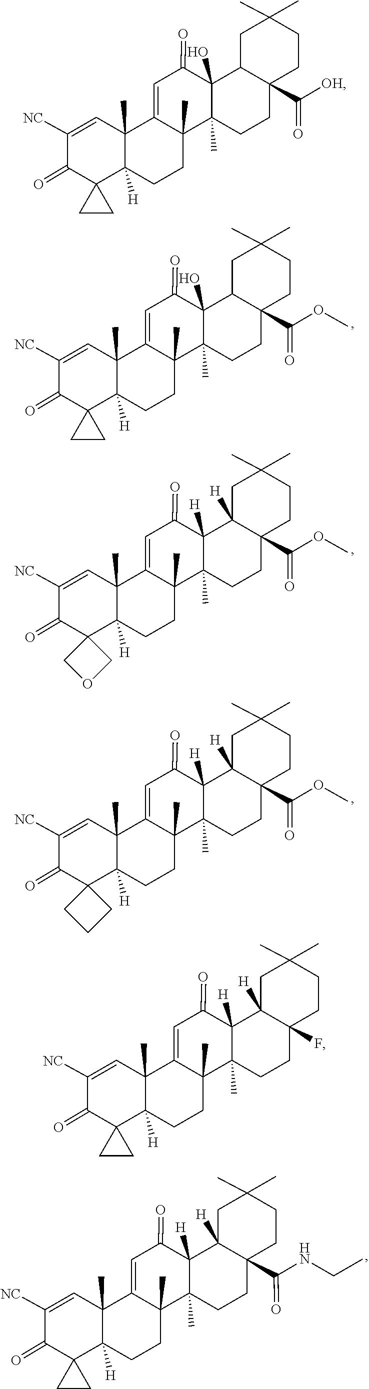

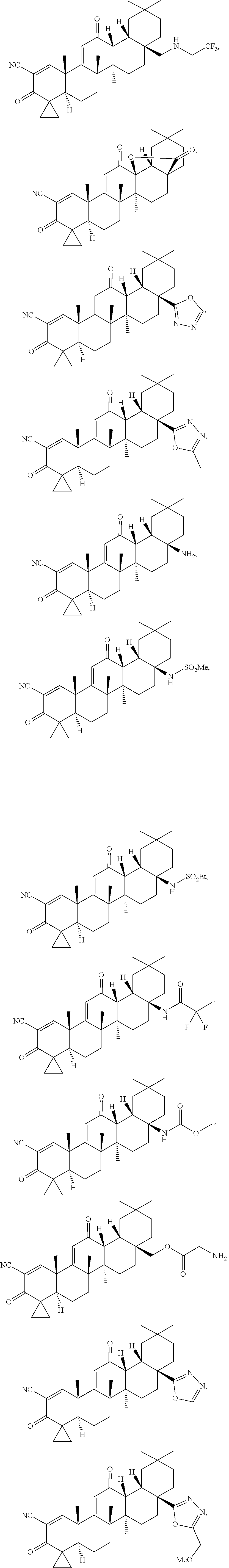

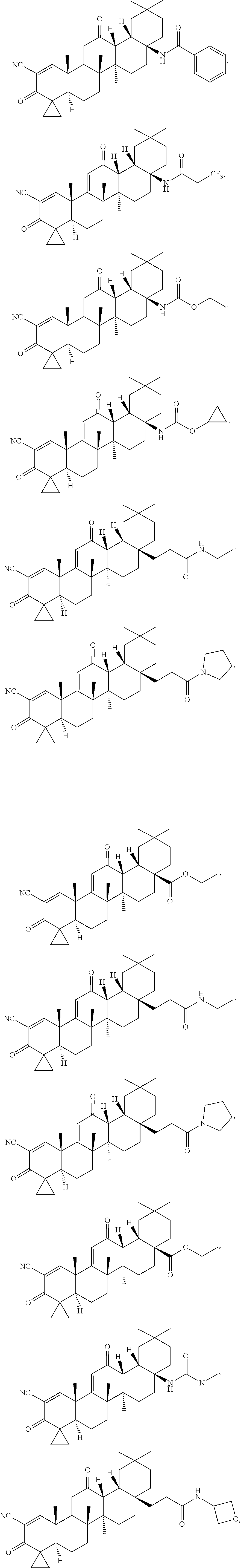

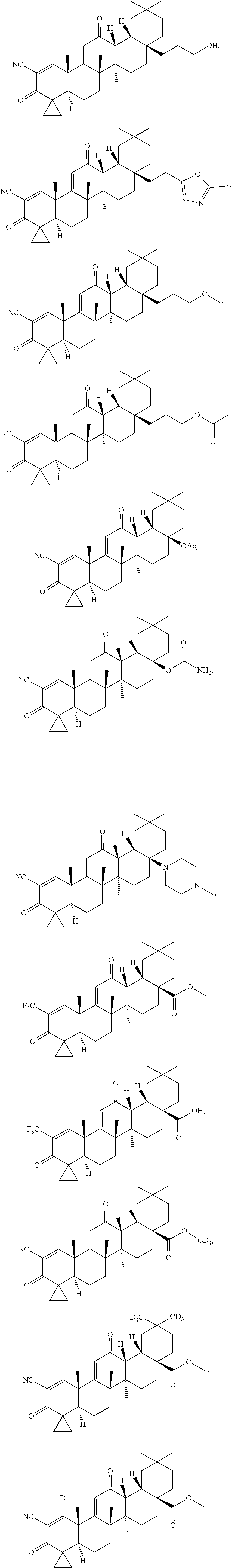

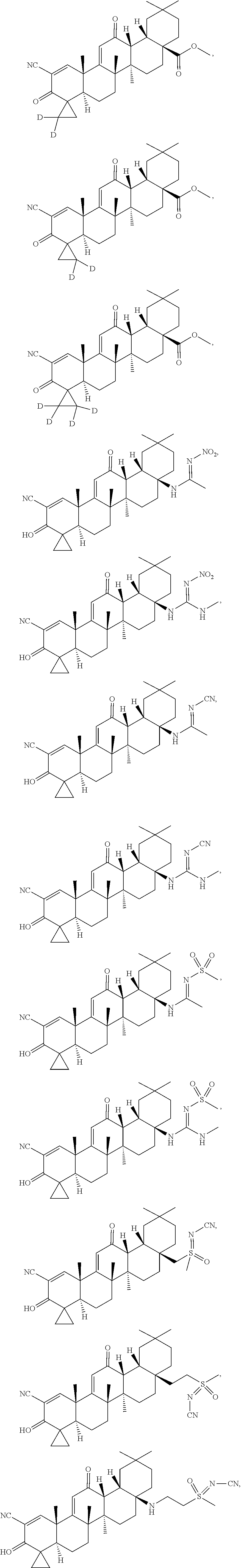

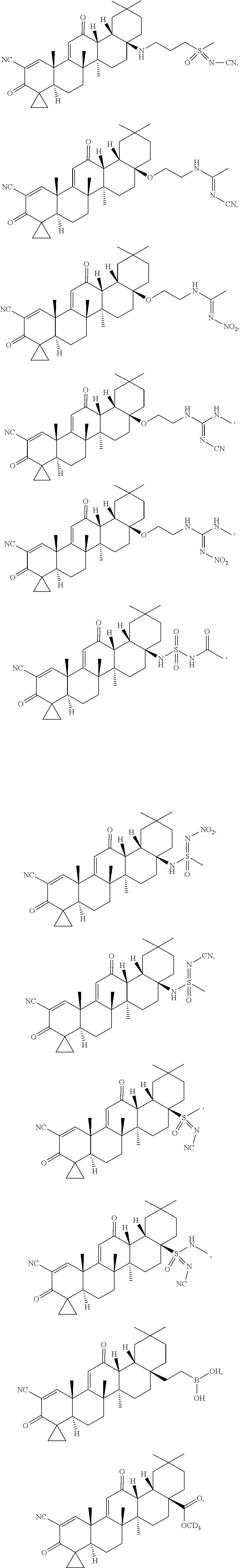

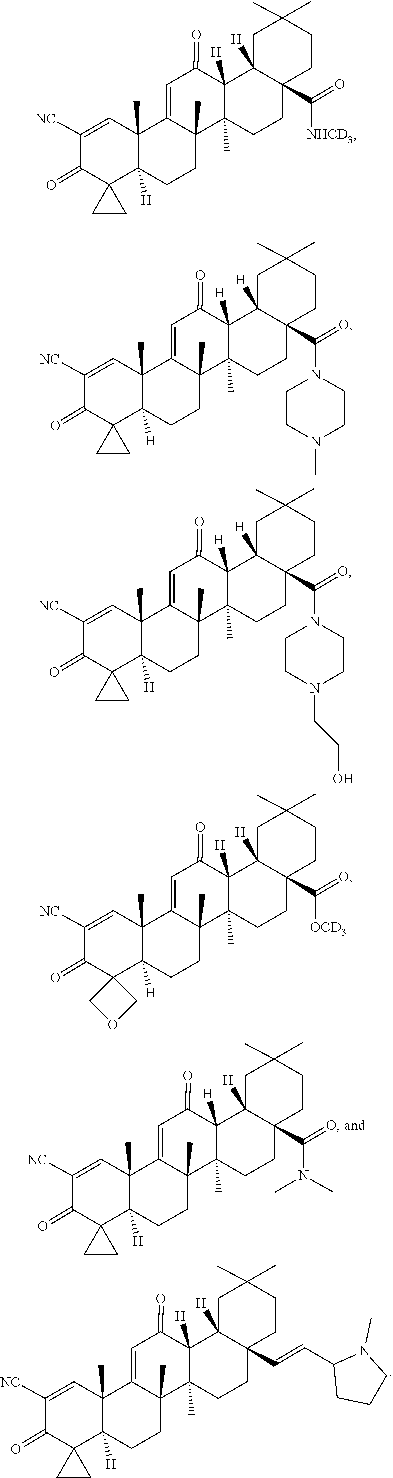

Disclosed herein is a compound, or a pharmaceutically acceptable salt, solvate, or stereoisomer thereof, selected from the group consisting of:

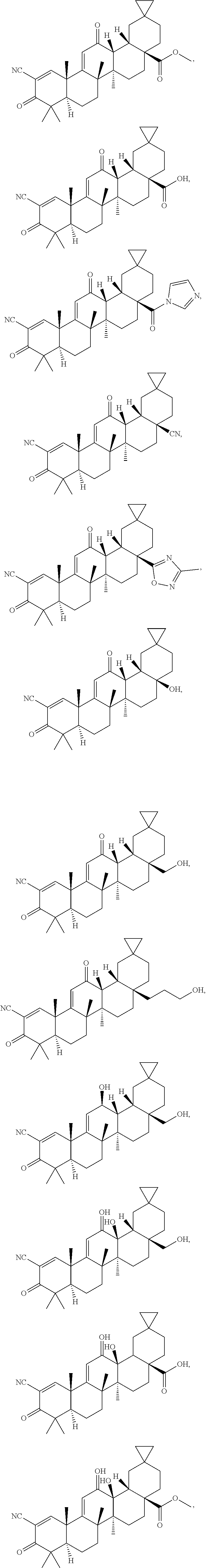

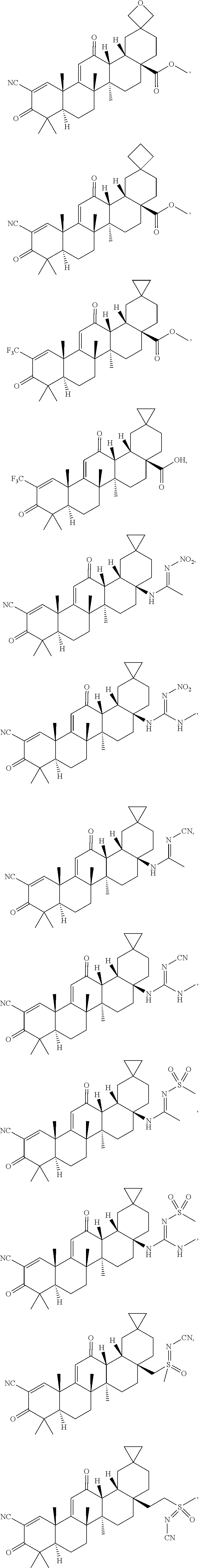

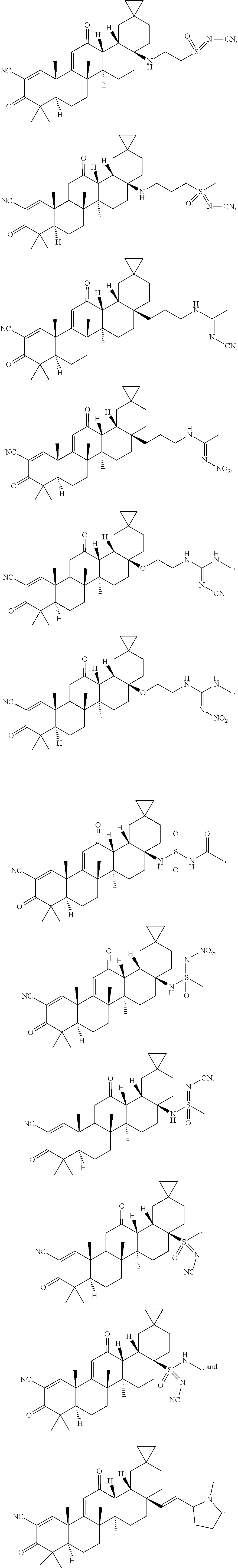

Disclosed herein is a compound, or a pharmaceutically acceptable salt, solvate, or stereoisomer thereof, selected from the group consisting of:

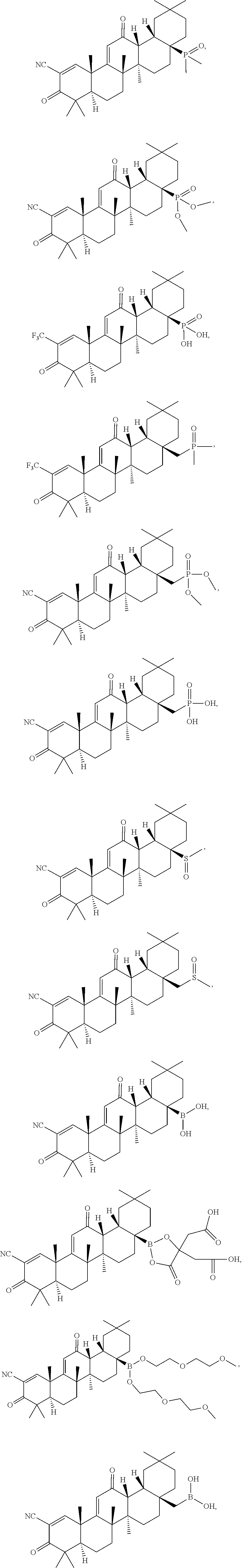

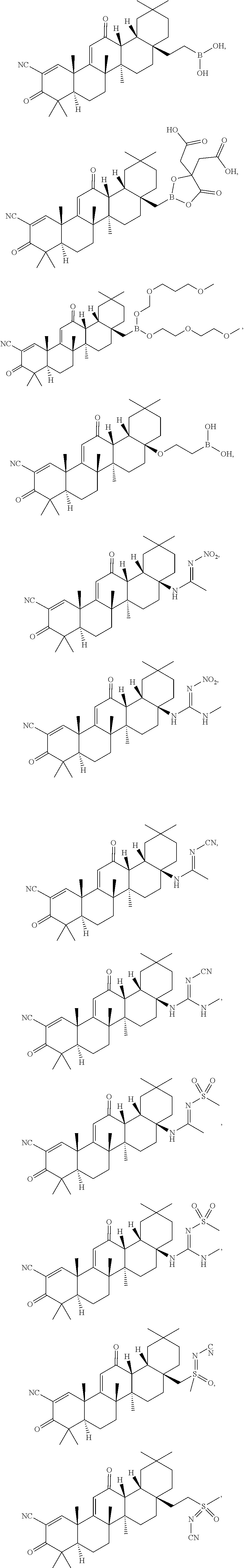

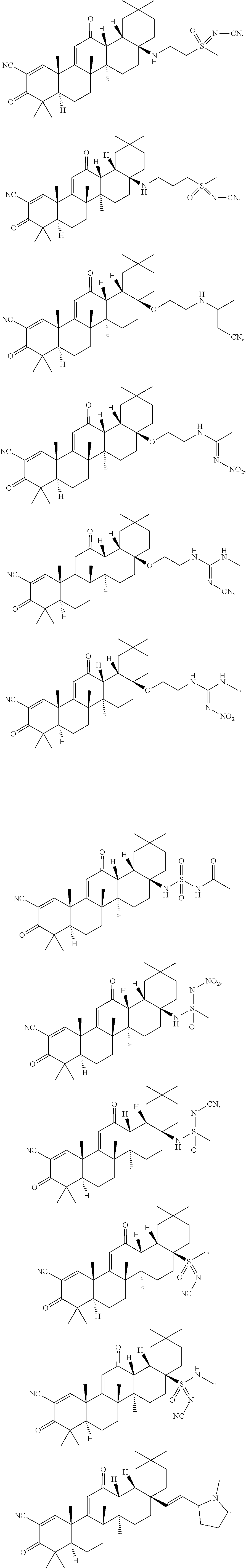

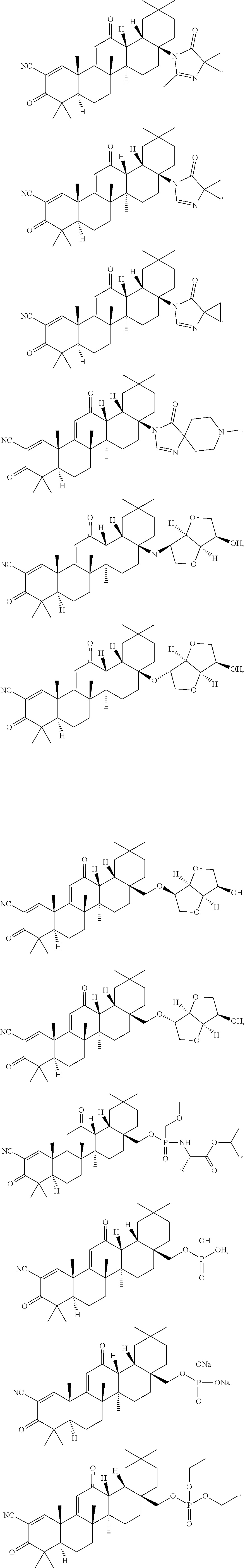

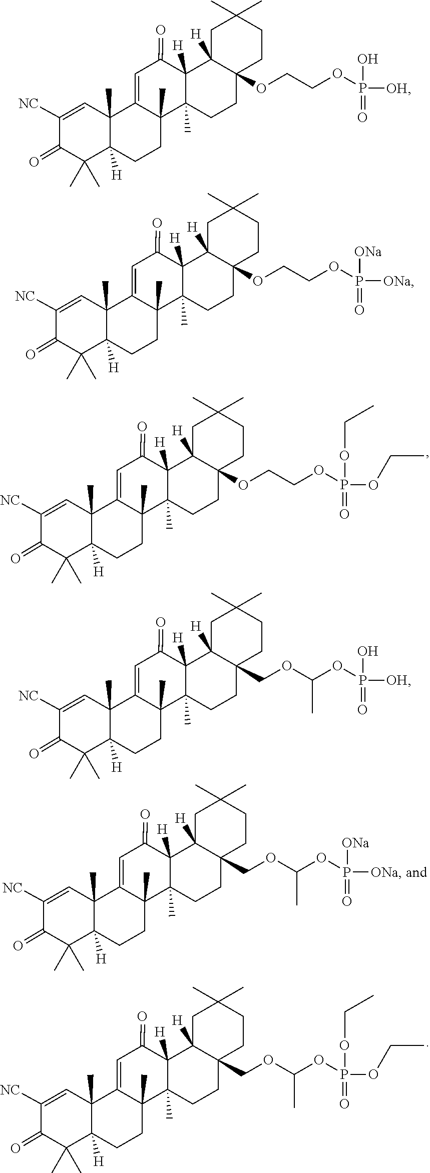

Disclosed herein is a compound, or a pharmaceutically acceptable salt, solvate, or stereoisomer thereof, selected from the group consisting of:

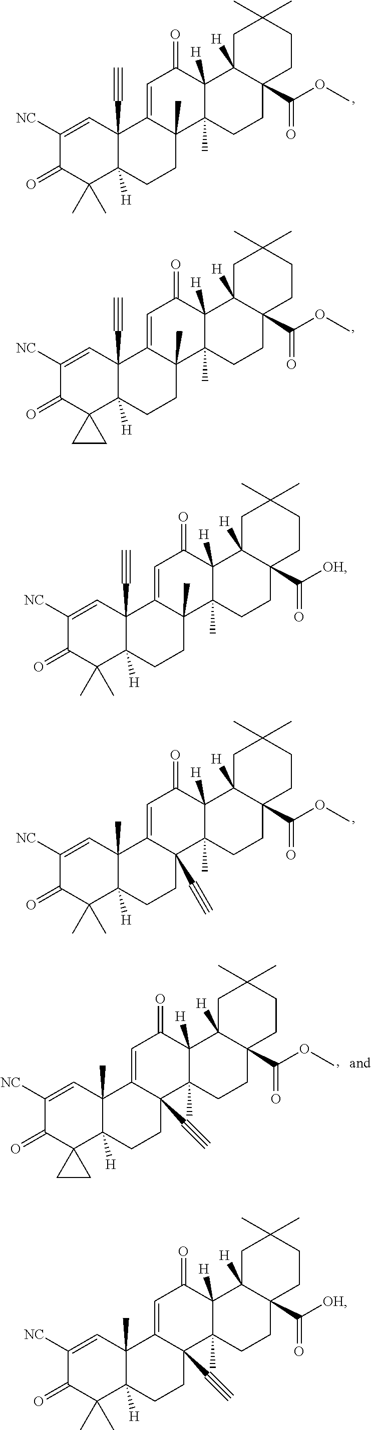

Disclosed herein is a compound, or a pharmaceutically acceptable salt, solvate, or stereoisomer thereof, selected from the group consisting of:

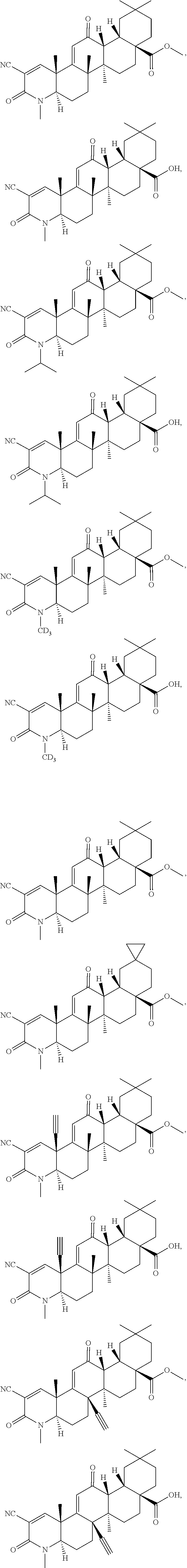

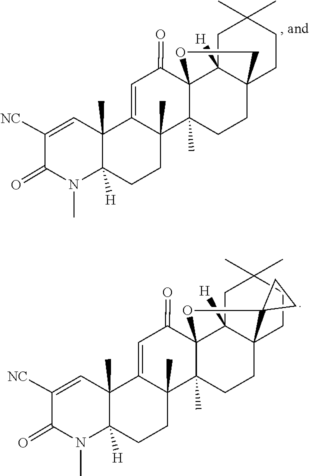

Disclosed herein is a compound, or a pharmaceutically acceptable salt, solvate, or stereoisomer thereof, selected from the group consisting of:

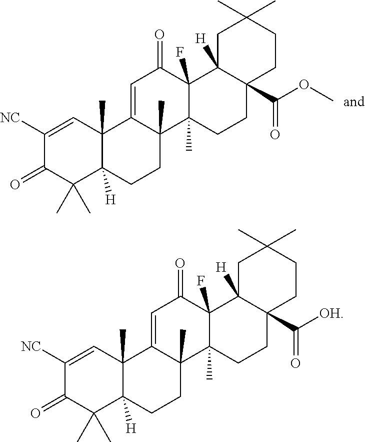

Disclosed herein is a compound, or a pharmaceutically acceptable salt, solvate, or stereoisomer thereof, selected from the group consisting of:

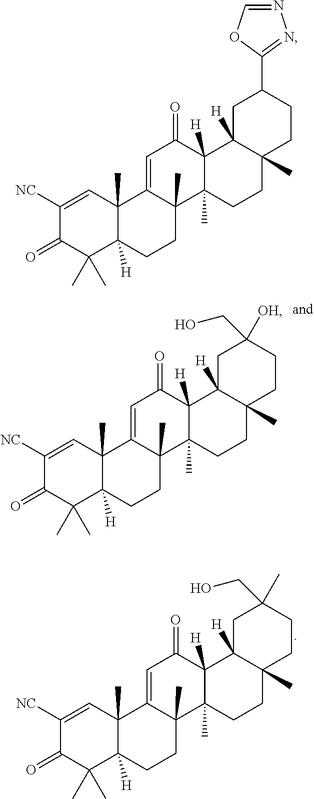

Disclosed herein is a compound, or a pharmaceutically acceptable salt, solvate, or stereoisomer thereof, selected from the group consisting of:

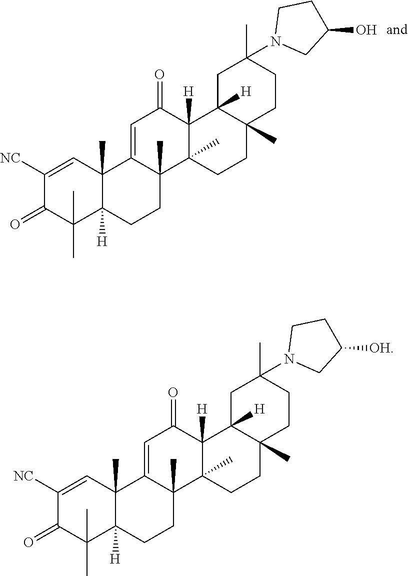

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are notable for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3 -phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quatemization of any basic nitrogen-containing groups they contain. In some embodiments, water or oil-soluble or dispersible products are obtained by such quatemization.

### Solvates

In some embodiments, the compounds described herein exist as solvates. This disclosure provides for methods of treating diseases by administering such solvates. This disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Accordingly, provided herein is a pharmaceutical composition comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal, epidural, or intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involves the administration of about 0.1 μg to about 50 mg of at least one compound described herein per kg body weight of the subject. For a 70 kg patient, dosages of about 10 μg to about 200 mg of the compound disclosed herein would be more commonly used, depending on a subject's physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In some embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 100 mg, about 150 mg, about 200 mg, about 250 mg, about 500 mg, about 750 mg, or about 1000 mg per day.

### Methods of Treatment

The compounds disclosed herein, or pharmaceutically acceptable salts, solvates, or stereoisomers thereof, are useful in the treatment or prevention of inflammation or diseases associated with inflammation.

Inflammation is a biological process that provides resistance to infectious or parasitic organisms and the repair of damaged tissue. Inflammation is commonly characterized by localized vasodilation, redness, swelling, and pain, the recruitment of leukocytes to the site of infection or injury, production of inflammatory cytokines such as TNF-α and IL-1, and production of reactive oxygen or nitrogen species such as hydrogen peroxide, superoxide, and peroxynitrite. In later stages of inflammation, tissue remodeling, angiogenesis, and scar formation (fibrosis) may occur as part of the wound healing process. Under normal circumstances, the inflammatory response is regulated and temporary and is resolved in an orchestrated fashion once the infection or injury has been dealt with adequately. However, acute inflammation can become excessive and life-threatening if regulatory mechanisms fail. Alternatively, inflammation can become chronic and cause cumulative tissue damage or systemic complications.

Many serious and intractable human diseases involve dysregulation of inflammatory processes, including diseases such as cancer, atherosclerosis, and diabetes, which were not traditionally viewed as inflammatory conditions. In the case of cancer, the inflammatory processes are associated with tumor formation, progression, metastasis, and resistance to therapy. Atherosclerosis, long viewed as a disorder of lipid metabolism, is now understood to be primarily an inflammatory condition, with activated macrophages playing an important role in the formation and eventual rupture of atherosclerotic plaques. Activation of inflammatory signaling pathways has also been shown to play a role in the development of insulin resistance, as well as in the peripheral tissue damage associated with diabetic hyperglycemia. Excessive production of reactive oxygen species and reactive nitrogen species such as superoxide, hydrogen peroxide, nitric oxide, and peroxynitrite is a hallmark of inflammatory conditions. Evidence of dysregulated peroxynitrite production has been reported in a wide variety of diseases.

Autoimmune diseases such as rheumatoid arthritis, lupus, psoriasis, and multiple sclerosis involve inappropriate and chronic activation of inflammatory processes in affected tissues, arising from dysfunction of self vs. non-self recognition and response mechanisms in the immune system. In neurodegenerative diseases such as Alzheimer's and Parkinson's diseases, neural damage is correlated with activation of microglia and elevated levels of pro-inflammatory proteins such as inducible nitric oxide synthase (iNOS). Chronic organ failure such as renal failure, heart failure, liver failure, and chronic obstructive pulmonary disease is closely associated with the presence of chronic oxidative stress and inflammation, leading to the development of fibrosis and eventual loss of organ function. Oxidative stress in vascular endothelial cells, which line major and minor blood vessels, can lead to endothelial dysfunction and is believed to be an important contributing factor in the development of systemic cardiovascular disease, complications of diabetes, chronic kidney disease, and other forms of organ failure, and a number of other aging-related diseases including degenerative diseases of the central nervous system and the retina.

Many other disorders involve oxidative stress and inflammation in affected tissues, including inflammatory bowel disease; inflammatory skin diseases; mucositis related to radiation therapy and chemotherapy; eye diseases such as uveitis, glaucoma, macular degeneration, and various forms of retinopathy; transplant failure and rejection; ischemia-reperfusion injury; chronic pain; degenerative conditions of the bones and joints including osteoarthritis and osteoporosis; asthma and cystic fibrosis; seizure disorders; and neuropsychiatric conditions including schizophrenia, depression, bipolar disorder, post-traumatic stress disorder, attention deficit disorders, autism-spectrum disorders, and eating disorders such as anorexia nervosa. Dysregulation of inflammatory signaling pathways is believed to be a major factor in the pathology of muscle wasting diseases including muscular dystrophy and various forms of cachexia.

A variety of life-threatening acute disorders also involve dysregulated inflammatory signaling, including acute organ failure involving the pancreas, kidneys, liver, or lungs, myocardial infarction or acute coronary syndrome, stroke, septic shock, trauma, severe burns, and anaphylaxis.

Many complications of infectious diseases also involve dysregulation of inflammatory responses. Although an inflammatory response can kill invading pathogens, an excessive inflammatory response can also be quite destructive and in some cases can be a primary source of damage in infected tissues. Furthermore, an excessive inflammatory response can also lead to systemic complications due to overproduction of inflammatory cytokines such as TNF-α and IL-1. This is believed to be a factor in mortality arising from severe influenza, severe acute respiratory syndrome, and sepsis.

The aberrant or excessive expression of either iNOS or cyclooxygenase-2 (COX-2) has been implicated in the pathogenesis of many disease processes. NO is a potent mutagen and nitric oxide can activate COX-2. Furthermore, there is a marked increase in iNOS in rat colon tumors induced by the carcinogen azoxymethane.

In one aspect, compounds disclosed herein are characterized by their ability to inhibit the production of nitric oxide in macrophage-derived RAW 264.7 cells induced by exposure to γ-interferon. They are further characterized by their ability to induce the expression of antioxidant proteins such as NQO1 and reduce the expression of pro-inflammatory proteins such as COX-2 and inducible nitric oxide synthase (iNOS). These properties are relevant to the treatment of a wide array of diseases and disorders involving oxidative stress and dysregulation of inflammatory processes including cancer, complications from localized or total-body exposure to ionizing radiation, mucositis resulting from radiation therapy or chemotherapy, autoimmune diseases, cardiovascular diseases including atherosclerosis, ischemia-reperfusion injury, acute and chronic organ failure including renal failure and heart failure, respiratory diseases, diabetes and complications of diabetes, severe allergies, transplant rejection, graft-versus-host disease, neurodegenerative diseases, diseases of the eye and retina, acute and chronic pain, degenerative bone diseases including osteoarthritis and osteoporosis, inflammatory bowel diseases, dermatitis and other skin diseases, sepsis, burns, seizure disorders, and neuropsychiatric disorders.

In another aspect, compounds disclosed herein are used in preventing or treating tissue damage or organ failure, acute and chronic, resulting from oxidative stress exacerbated by inflammation. Examples of diseases that fall in this category include: heart failure, liver failure, transplant failure and rejection, renal failure, pancreatitis, fibrotic lung diseases (cystic fibrosis, COPD, and idiopathic pulmonary fibrosis, among others), diabetes (including complications), atherosclerosis, ischemia- reperfusion injury, glaucoma, stroke, autoimmune disease, autism, macular degeneration, and muscular dystrophy.

In some embodiments, the compounds disclosed herein are generally applied to the treatment of inflammatory conditions, such as sepsis, dermatitis, autoimmune disease, and osteoarthritis. In one aspect, the compounds disclosed herein are used to treat inflammatory pain and/or neuropathic pain, for example, by inducing Nrf2 and/or inhibiting NF-KB.

In some embodiments, the compounds disclosed herein are used in the treatment and prevention of diseases such as cancer, inflammation, Alzheimer's disease, Parkinson's disease, multiple sclerosis, autism, amyotrophic lateral sclerosis, Huntington's disease, autoimmune diseases such as rheumatoid arthritis, lupus, Crohn's disease, and psoriasis, inflammatory bowel disease, other diseases whose pathogenesis is believed to involve excessive production of either nitric oxide or prostaglandins, and pathologies involving oxidative stress alone or oxidative stress exacerbated by inflammation.

In some embodiments, the compounds disclosed herein are used in the treatment and prevention of diseases such as NASH. In some embodiments, the compounds disclosed herein are used in the treatment and prevention of diseases such as irritable bowel disease. In some embodiments, the compounds disclosed herein are used in the treatment and prevention of diabetic nephropathy. In some embodiments, the compounds disclosed herein are used in the treatment and prevention of chronic kidney disease.

In one aspect, the compounds disclosed herein are used to control the production of pro-inflammatory cytokines within the cell by selectively activating regulatory cysteine residues (RCRs) on proteins that regulate the activity of redox-sensitive transcription factors. Activation of RCRs by cyPGs has been shown to initiate a pro-resolution program in which the activity of the antioxidant and cytoprotective transcription factor Nrf2 is potently induced and the activities of the pro-oxidant and pro-inflammatory transcription factors NF-κB and the STATs are suppressed. In some embodiments, this increases the production of antioxidant and reductive molecules (NQO1, HO-1, SOD1, γ-GCS) and decreases oxidative stress and the production of pro- oxidant and pro-inflammatory molecules (iNOS, COX-2, TNF-α). In some embodiments, the compounds disclosed herein cause the cells that host the inflammatory event to revert to a non-inflammatory state by promoting the resolution of inflammation and limiting excessive tissue damage to the host.

### Combination Therapy

In certain instances, the compound described herein, or a pharmaceutically acceptable salt, solvate, or stereoisomer thereof, is administered in combination with a second therapeutic agent.

In some embodiments, the benefit experienced by a patient is increased by administering one of the compounds described herein with a second therapeutic agent (which also includes a therapeutic regimen) that also has therapeutic benefit.

In one specific embodiment, a compound described herein, or a pharmaceutically acceptable salt, solvate, or stereoisomer thereof, is co-administered with a second therapeutic agent, wherein the compound described herein, or a pharmaceutically acceptable salt, solvate, or stereoisomer thereof, and the second therapeutic agent modulate different aspects of the disease, disorder, or condition being treated, thereby providing a greater overall benefit than administration of either therapeutic agent alone.

In any case, regardless of the disease, disorder, or condition being treated, the overall benefit experienced by the patient is simply additive of the two therapeutic agents or the patient experiences a synergistic benefit.

In certain embodiments, different therapeutically effective dosages of the compounds disclosed herein will be utilized in formulating a pharmaceutical composition and/or treatment regimen when the compounds disclosed herein are administered in combination with a second therapeutic agent. Therapeutically effective dosages of drugs and other agents for use in combination treatment regimens are optionally determined by means similar to those set forth herein for the compounds described herein. Furthermore, the methods of prevention/treatment described herein encompass the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt, solvate, or stereoisomer thereof, is initiated prior to, during, or after treatment with a second agent described herein, and continues until any time during treatment with the second agent or after termination of treatment with the second agent. It also includes treatments in which a compound described herein, or a pharmaceutically acceptable salt, solvate, or stereoisomer thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought is modified in accordance with a variety of factors (e.g. the disease, disorder, or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated, and so forth. In additional embodiments, when co-administered with a second therapeutic agent, the compound provided herein is administered either simultaneously with the second therapeutic agent, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt, solvate, or stereoisomer thereof, as well as combination therapies, are administered before, during, or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month, or about 5 years.

In some embodiments, the compound described herein, or a pharmaceutically acceptable salt, solvate, or stereoisomer thereof, is administered in combination with an adjuvant. In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced).

### EXAMPLES

### Intermediate 1: (2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicene-2-carboxylic acid

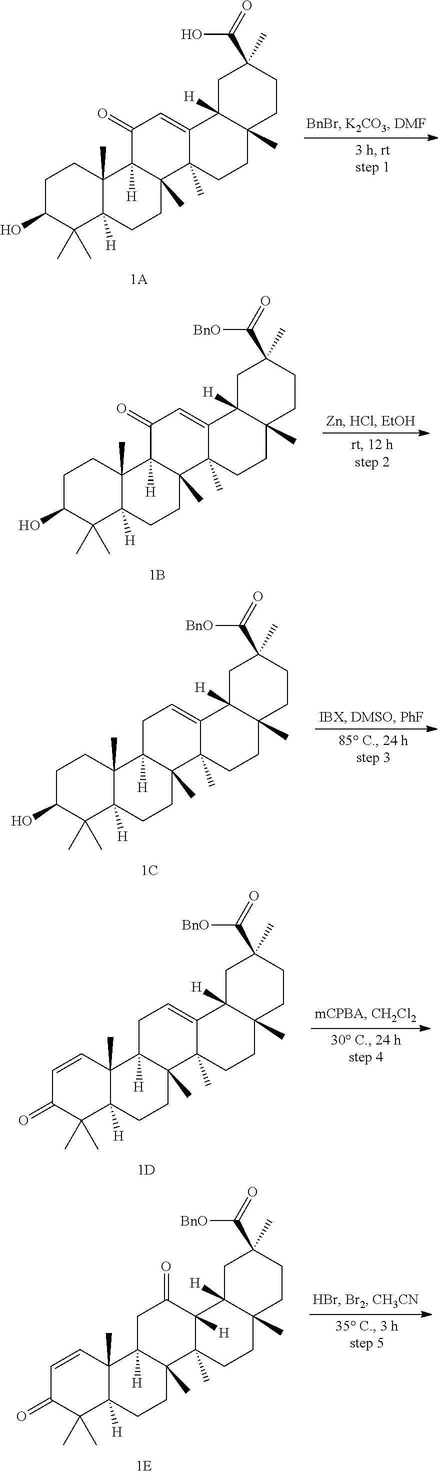

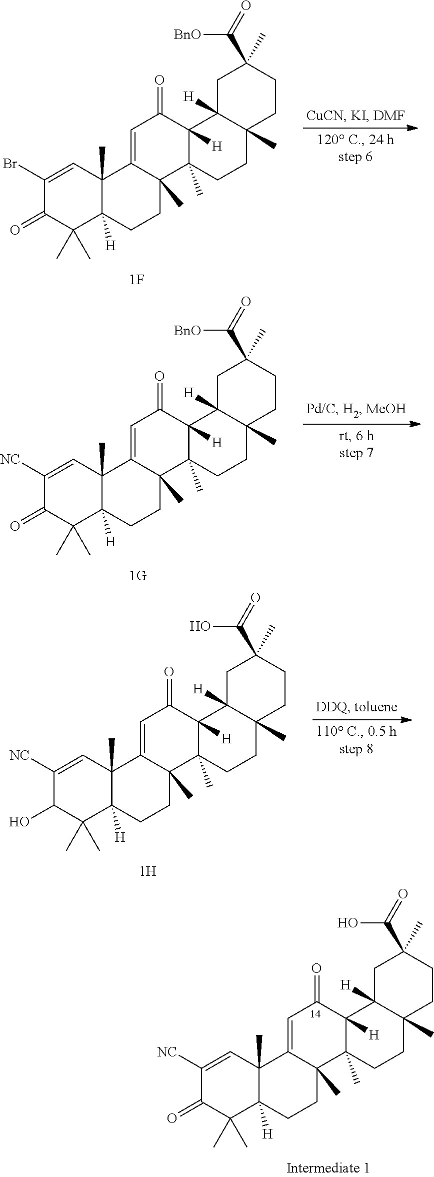

### Step 1: benzyl (2S,4aS,6aS,6bR,8aR,10S,12aS,12bR,14bR)-10-hydroxy-2,4a,6a,6b,9,9,12a-heptamethyl-13-oxo-1,2,3,4,4a,5,6,6a,6b,7,8,8a,9,10,11,12,12a,12b,13,14b-icosahydropicene-2-carboxylate (1B)

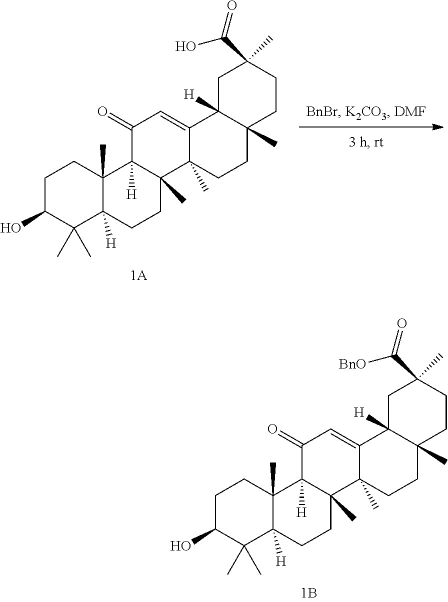

To a stirred solution of glycyrrhetnic acid (1A) (470 g, 1.0 mol) in dimethyl formamide (3 L) was added powdered potassium carbonate (414 g, 3.0 mol) at 0° C. followed by slow addition of benzyl bromide (205 g, 1.2 mol). After completion of addition, the reaction mixture was allowed to warm to room temperature and stirred for 12 h. The reaction was quenched by addition of water (5 L) and filtered. The residue was washed with water (2 L) and dried on vacuum to obtain 1B (500 g, 89%) as a white solid, which was used directly for the next step without further purifications.

### Step 2: benzyl (2S,4aS,6aS,6bR,8aR,10S,12aR,12bR,14bR)-10-hydroxy-2,4a,6a,6b,9,9,12a-heptamethyl-1,2,3,4,4a,5,6,6a,6b,7,8,8a,9,10,11,12,12a,12b,13,14b-icosahydropicene-2-carboxylate (1C)

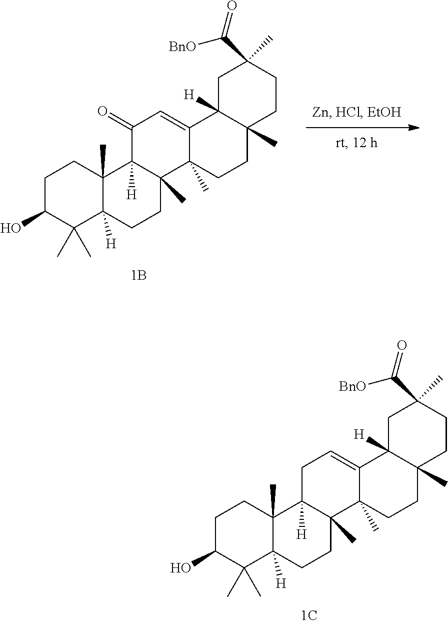

Compound (1B) (250 g, 446 mmol) and zinc powder (583 g, 8.9 mol) were dissolved in EtOH (3 L), then concentrated hydrochloric acid (1.5 L) was added to the mixture slowly at 0° C. After completion of addition, the reaction was stirred for 12 h at room temperature. Then the reaction mixture was filtered, extracted with methylene chloride (3 L×3), and the organics were dried over Na₂SO₄ and the solvent was removed to give crude product, which was purified by flash chromatography to afford 1C (180 g, 74%) as a white solid. ¹H NMR (400 MHz, CDCl₃) δ 7.42 7.27 (m, 5H), 5.23 5.04 (m, 3H), 3.24-3.19 (m, 1H), 2.06-1.93 (m, 2H), 1.93-1.82 (m, 4H), 1.82-1.70 (m, 1H), 1.69 1.49 (m, 7H), 1.49-1.36 (m, 3H), 1.37-1.21 (m, 5H), 1.14 (s, 3H), 1.13 (s, 3H), 0.99 (s, 3H), 0.95 (s, 3H), 0.93 (s, 3H), 0.92-0.82 (m, 2H), 0.79 (s, 3H), 0.74 (s, 3H).

### Step 3: benzyl (2S,4aS,6aS,6bR,8aR,12aR,12bR,14bR)-2,4a,6a,6b,9,9,12a-heptamethyl-10-oxo-1,2,3,4,4a,5,6,6a,6b,7,8,8a,9,10,12a,12b,13,14b-octadecahydropicene-2-carboxylate (1D)

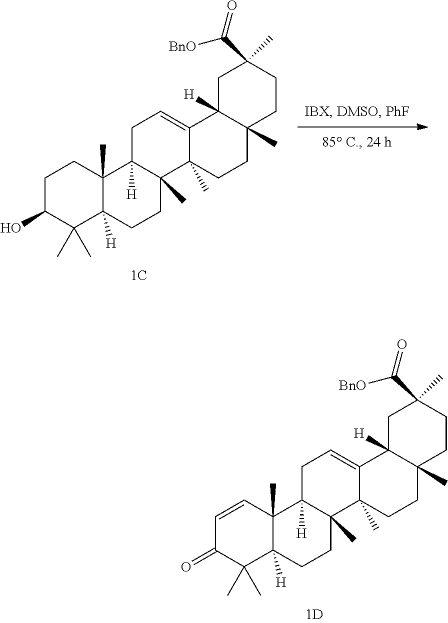

To a stirred suspension of 1C (55 g, 100 mmol) obtained above in anhydrous dimethyl sulfoxide (400 mL) was added iodoxybenzoic acid (112 g, 0.4 mol) and fluorobenzene (25 mL). The resulting suspension was heated to 85° C. under nitrogen for 24 hours. After the completion of the reaction, it was quenched with 20% aqueous sodium thiosulfate (500 mL). The resulting mixture was extracted with methylene chloride (4×500 mL), the combined organic extracts were washed with saturated NaHCO₃ (500 mL) and brine (500 mL), and dried over Na₂SO₄. The solvent was removed to give the crude product as yellowish solid, which was subjected to flash column chromatography to give pure 1D (43 g, 78%) as a white solid.

### Step 4: benzyl (2S,4aS,6aR,6bR,8aR,12aR,12bR,14aR,14bS)-2,4a,6a,6b,9,9,12a-heptamethyl- 10,14-dioxo-1,2,3,4,4a,5,6,6a,6b,7,8,8a,9,10,12a,12b,13,14,14a,14b-icosahydropicene-2-carboxylate (1E)

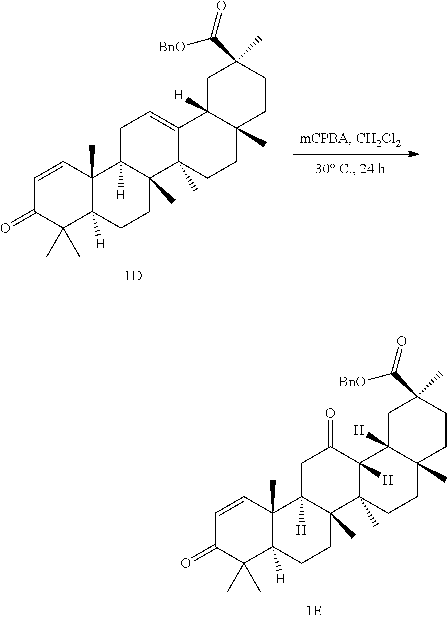

To a stirred solution of 1D (43 g, 78 mmol) in methylene chloride (400 mL) was slowly added m-chloroperbenzoic acid (20.4 g, 85% purity, 100 mmol) at 0° C. After the completion of addition, the reaction was allowed to warm to room temperature and kept stirring for 24 hours. After the completion of the reaction, the reaction mixture was diluted with methylene chloride (300 mL), and the resulting mixture was washed with 20% aqueous sodium thiosulfate (2×300 mL), 10% potassium carbonate (2×300 mL), and brine (300 mL). The organics were dried over Na₂SO₄ and the solvent was removed to give crude product 1E as yellowish solid, which was used directly for the next step without further purifications.

### Step 5: benzyl (2S,4aS,6aR,6bS,8aR,12aR,14aR,14bS)-11-bromo-2,4a,6a,6b,9,9,12a-heptamethyl-10,14-dioxo-1,2,3,4,4a,5,6,6a,6b,7,8,8a,9,10,12a,14,14a,14b-octadecahydropicene-2-carboxylate (1F)

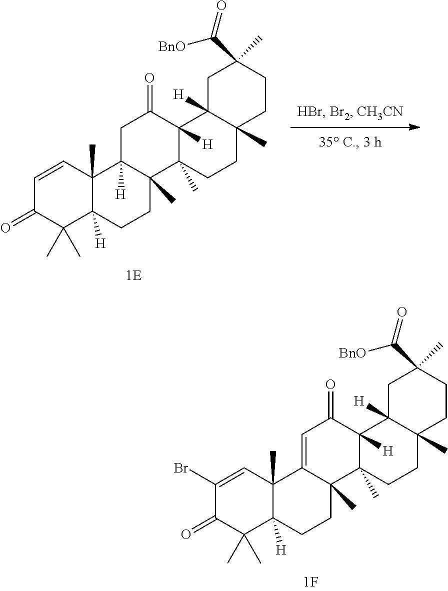

To the solution of 1E obtained above in acetic acid (200 mL) was added dropwise hydrobromic acid (4.5 mL, 39 mmol) at room temperature. The reaction mixture was then heated to 35° C., and bromine (10 mL, 0.2 mol) was thus added dropwise. The resulting reaction mixture was kept stirring for another 3 h. After completion of the reaction, the acid was removed under vacuum. And the residue was then quenched with 20% aqueous sodium thiosulfate (200 mL), and extracted with methylene chloride (4×200 mL). The combined organic extracts were washed with saturated sodium bicarbonate (2×200 mL), brine (1×200 mL), and dried over Na₂SO₄. The residue was subjected to flash column chromatography to give pure 1F (14.8 g, 30% from 1D) as a yellowish solid. LC-MS (ESI) m/z: 635.3/637.3 [M+H]⁺. ¹H NMR (400 MHz, CDCl₃) δ 7.83 (s, 1H), 7.50 (d, J=7.2 Hz, 2H), 7.43 7.28 (m, 3H), 6.04 (s, 1H), 5.20 (q, J=12.5 Hz, 2H), 3.04 (d, J=4.7 Hz, 1H), 2.37 2.25 (m, 1H), 2.14-2.08 (m, 1H), 2.05 1.90 (m, 2H), 1.90-1.65 (m, 5H), 1.59-1.50 (m, 2H), 1.48 (s, 3H), 1.46 (s, 3H), 1.36 1.28 (m, 2H), 1.26 (s, 3H), 1.26-1.20 (m, 1H), 1.19 (s, 3H), 1.13 (s, 3H), 1.10-1.06(m, 1H), 1.01 (s, 3H), 0.97-0.91 (m, 1H), 0.90 (s, 3H).

### Step 6: benzyl (2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicene-2-carboxylate (1G)

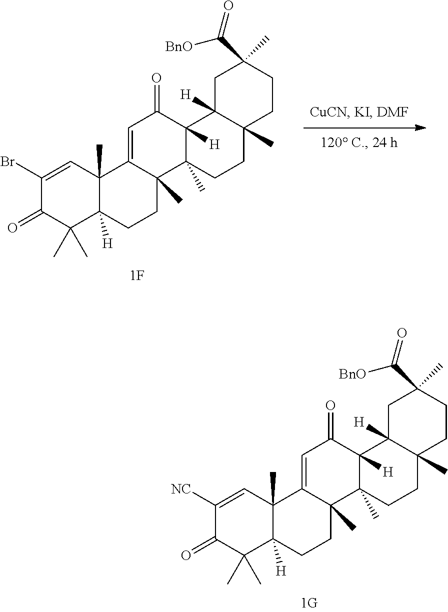

To a stirred solution of 1F (14.8 g, 23.3 mmol) in anhydrous dimethyl formamide (150 mL) was added copper (I) cyanide (3.1 g, 35 mmol) and potassium iodide (0.77 g, 4.7 mmol), and the resulting reaction mixture was heated to 120° C. for 24 h. After the completion of reaction, it was cooled to room temperature, quenched with water (300 mL), and diluted with ethyl acetate (300 mL). The organic phase was washed with saturated NaHCO₃ (2×200 mL), brine (200 mL), and dried over Na₂SO₄. Removal of solvent and the residue was subjected to flash column chromatography to give pure 1G (10.4 g, 77%) as a yellowish solid. LC-MS (ESI) m/z: 582.4 [M+H]⁺

### Step 7: (2S,4aS,6aR,6bS,8aR,12aS,14aR,14bS)-11-cyano-10-hydroxy-2,4a,6a,6b,9,9,12a-heptamethyl-14-oxo-1,2,3,4,4a,5,6,6a,6b,7,8,8a,9,12,12a,14,14a,14b-octadecahydropicene-2-carboxylic acid (1H)

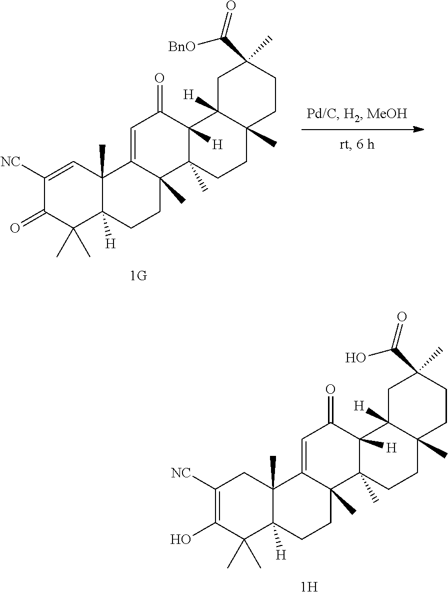

To a stirred solution of 1G (10.4 g, 18 mmol) dissolved in THF(100 mL), palladium carbon (2 g) was added and the mixture was stirred for 6 h under hydrogen atmosphere. After completion of the reaction, the mixture was filtered and concentrated under reduced pressure to obtain 1H as a brown solid, which was used directly for the next step without further purifications. LC-MS (ESI) m/z: 494.3 [M+H]⁺

### Step 7: (2S,4aS,6aR,6bS,8aR,12aS,14aR,14bS)-11-cyano-2,4a,6a,6b,9,9,12a-heptamethyl- 10,14-dioxo-1,2,3,4,4a,5,6,6a,6b,7,8,8a,9,10,12a,14,14a,14b-octadecahydropicene-2-carboxylic acid (intermediate 1)

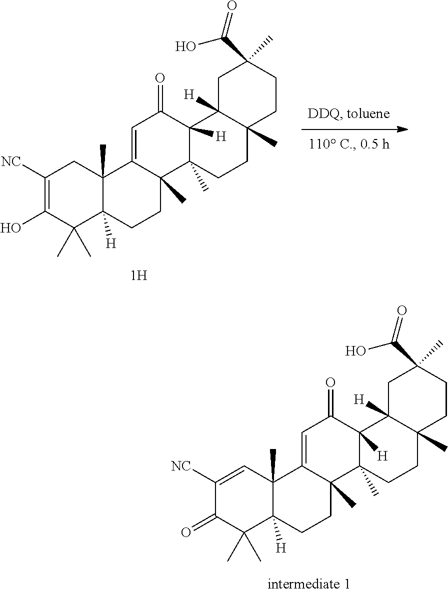

A solution of 1H obtained above and DDQ (4.1 g, 18 mmol) in dry toluene (80 mL) was heated under reflux for 30 min. After completion of the reaction, the mixture was filtered and concentrated under reduced pressure. The residue was subjected to flash column chromatography to give pure intermediate 1 (10.4 g, 77%) as a yellowish solid. LC-MS (ESI) m/z: 492.3 [M+H]⁺. ¹H NMR (400 MHz, CDCl₃) δ 8.07 (s, 1H), 6.06 (s, 1H), 3.18 (d, J=4.5 Hz, 1H), 2.26-2.19 (m, 2H), 2.03-1.70 (m, 6H), 1.70-1.53 (m, 3H), 1.52 (s, 3H), 1.50 (s, 3H), 1.43 1.30 (m, 2H), 1.30 1.23 (m, 5H), 1.20 (s, 3H), 1.18 (s, 3H), 1.13-1.08(m, 1H), 1.02 (s, 3H), 0.99 (s, 3H).

### Intermediate 2: tert-butyl(((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)carbamate

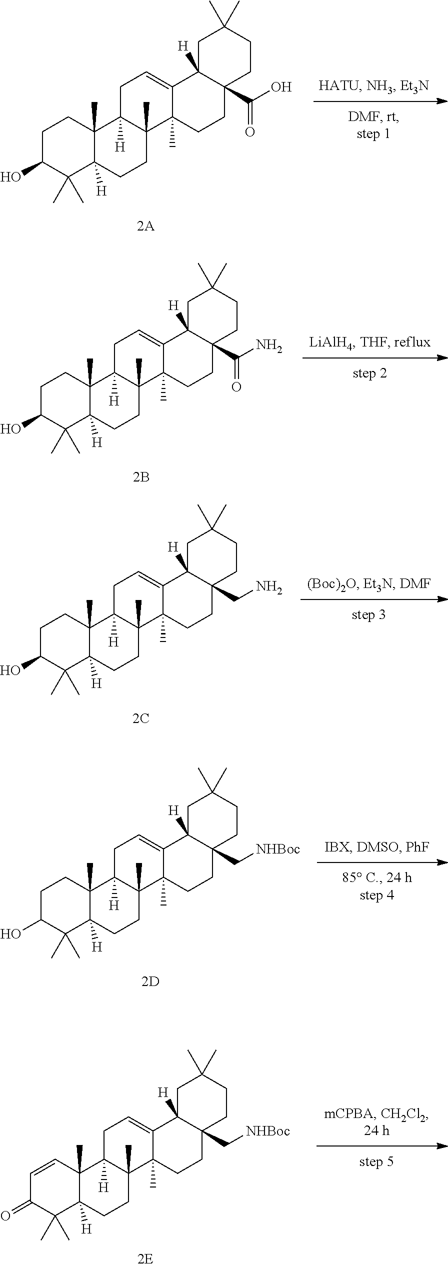

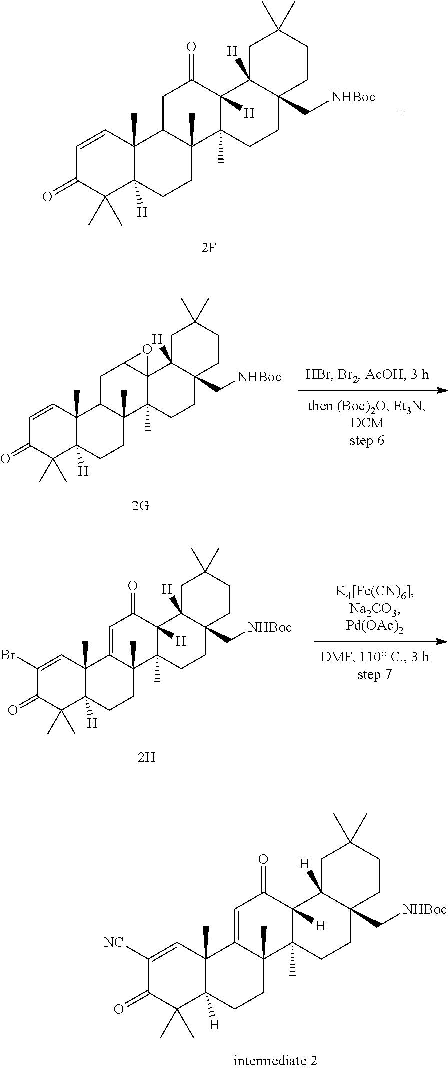

### Step 1: (4aS,6aS,6bR,8aR,10S,12aR,12bR,14bS)-10-hydroxy-2,2,6a,6b,9,9,12a-heptamethyl-1,2,3,4,4a,5,6,6a,6b,7,8,8a,9,10,11,12,12a,12b,13,14b-icosahydropicene-4a-carboxamide(2B)

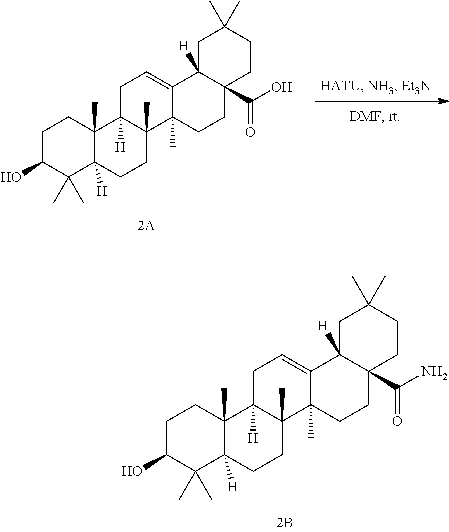

To a solution of oleanolic acid 2A (92 g, 0.2 mol) in DMF (500 mL) were successively added HATU (91.2 g, 0.24 mol) and Et₃N (41 g, 0.4 mol). The reaction mixture was stirred at room temperature for 0.5 h, then the mixture was added NH₃ (42 mL, 7M in MeOH) and stirred at room temperature for 3 h. The mixture was diluted with water (1 L) and filtered. The residue was washed with water (1 L) and dried on vacuum to obtain 2B as a white solid, which was used directly for the next step without further purifications. LC-MS (ESI) m/z 456.4 [M+H]⁺

### Step 2: (3S,4aR,6aR,6bS,8aS,12aS,14aR,14bR)-8a-(aminomethyl)-4,4,6a,6b,11,11,14b- heptamethyl-1,2,3,4,4a,5,6,6a,6b,7,8,8a,9,10,11,12,12a,14,14a,14b-icosahydropicen-3-ol (2C)

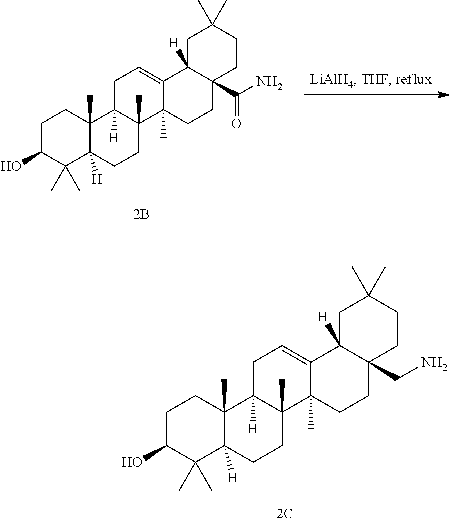

To a solution of 2B (90 g, 197 mmol) in THF (600 mL) were added LiAlH₄ (30 g, 0.79 mol) in portion under 0° C. Then the reaction mixture was heated to 60° C. for 4 h. After the completion of the reaction, it was allowed to cool to 0° C., the mixture was quenched successively with water (30 mL), 15% NaOH (60 mL) and water (90 mL). The mixture was filtered and filtrate was dried over Na₂SO₄. The solvent was removed to give the crude product 2C as white solid, which was used directly for the next step without further purifications. LC-MS (ESI) m/z 442.4 [M+FI]⁺

### Step 3: tert-butyl(((4aS,6aS,6bR,8aR,12aR,12bR,14bS)-10-hydroxy-2,2,6a,6b,9,9,12a- heptamethyl-1,2,3,4,4a,5,6,6a,6b,7,8,8a,9,10,11,12,12a,12b,13,14b-icosahydropicen-4a-yl)methyl)carbamate (2D)

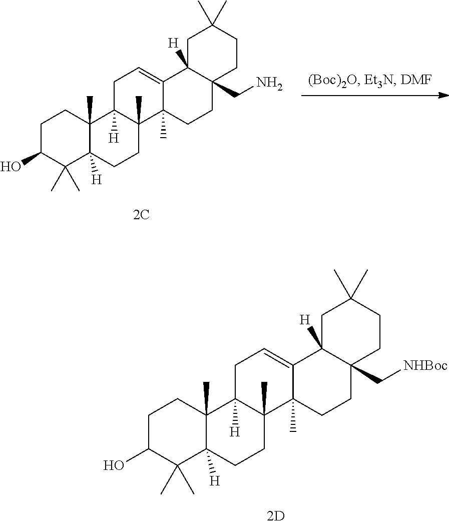

To a stirred suspension of 2C (80 g, 0.18 mol) obtained above in DCM (500 mL) was added Et₃N (36.4 g, 0.36 mol). The resulting suspension was added dropwise Di-tert-butyl pyrocarbonate (47 g in 200 ml DCM). After the completion of the reaction, the solvent was removed to give the crude product, which was subjected to flash column chromatography to give pure 2D (72 g, 73%) as a white solid.

### Step 4: tert-butyl(04aS,6aS,6bR,8aR,12aR,12bR,14bS)-2,2,6a,6b,9,9,12a-heptamethyl-10-oxo-1,2,3,4,4a,5,6,6a,6b,7,8,8a,9,10,12a,12b,13,14b-octadecahydropicen-4a-yl)methyl)carbamate (2E)

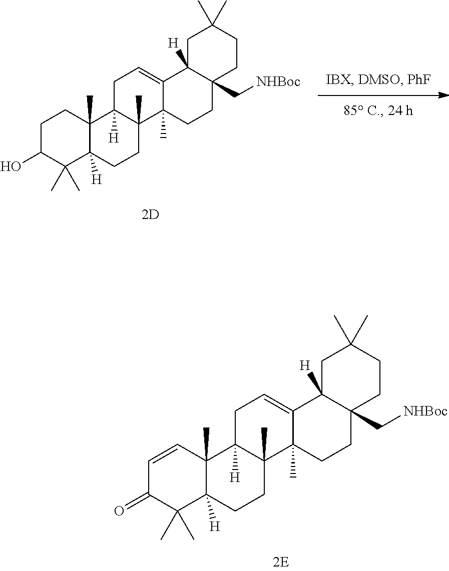

To a stirred suspension of 2D (36 g, 66 mmol) obtained above in anhydrous dimethyl sulfoxide (250 mL) was added iodoxybenzoic acid (55.8 g, 0.2 mol) and fluorobenzene (15 mL). The resulting suspension was heated to 85° C. under nitrogen for 24 h. After the completion of the reaction, it was quenched with 20% aqueous sodium thiosulfate (300 mL). The resulting mixture was extracted with methylene chloride (4×300 mL), the combined organic extracts were washed with saturated NaHCO₃ (300 mL) and brine (300 mL), and dried over Na₂SO₄. The solvent was removed to give the crude product, which was subjected to flash column chromatography to give pure 2E (27 g, 76%) as a white solid. LC-MS (ESI) m/z 438.4 [M-Boc+H]⁺

### Step 5: tert-butyl(((4aS,6aR,6bR,8aR,12aR,14aR,14bS)-2,2,6a,6b,9,9,12a-heptamethyl-10,14-dioxo-1,3,4,5,6,6a,6b,7,8,8a,9,10,12a,12b,13,14,14a,14b-octadecahydropicen-4a(2H)-yl)methyl)carbamate (2F)

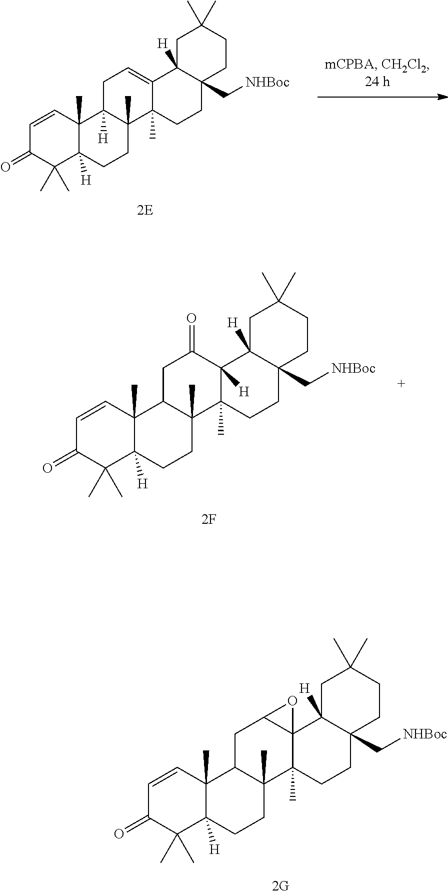

To a stirred solution of 2E (27 g, 50 mmol) in methylene chloride (300 mL) was slowly added mchloroperbenzoic acid (14.2 g, 70 mmol) at 0° C. After the completion of addition, the reaction was allowed to warm to room temperature and kept stirring for 24 hours. After the completion of the reaction, the reaction mixture was diluted with methylene chloride (300 mL), and the resulting mixture was washed with 20% aqueous sodium thiosulfate (3×200 mL), 10% potassium carbonate (2×200 mL), and brine (200 mL). The organics were dried over Na₂SO₄ and the solvent was removed to give crude mixture of 2F and 2G as yellowish solid, which was used directly for the next step without further purifications.

### Step 6: tert-butyl(((4aS,6aR,6bS,8aR,12aR,14aR,14bS)-11-bromo-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)carbamate (2H)

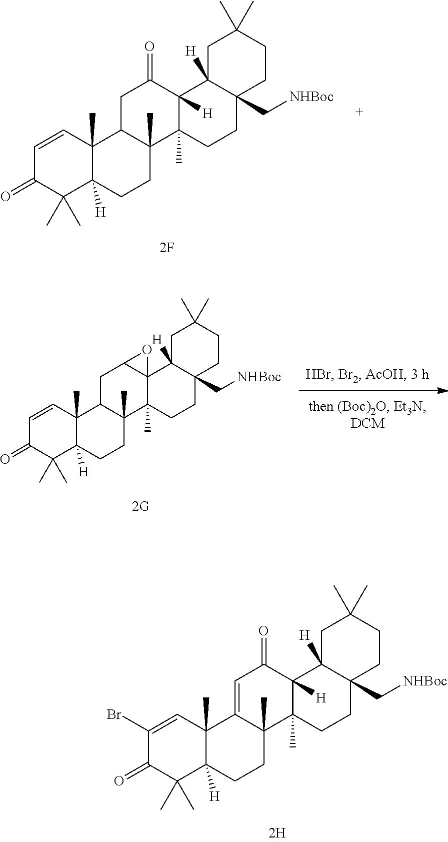

To the solution of 2F and 2G obtained above in acetic acid (150 mL) was added dropwise hydrobromic acid (2.9 mL, 25 mmol) at room temperature. The reaction mixture was then heated to 35° C., and bromine (6.2 mL, 0.12 mol) was thus added dropwise. The resulting reaction mixture was kept stirring for another 3 h. After completion of the reaction, the acid was removed under vacuum. And the residue was then quenched with 20% aqueous sodium thiosulfate (100 mL), and extracted with methylene chloride (4×100 mL). The combined organic extracts were washed with saturated sodium bicarbonate (2×200 mL), brine (1×200 mL), and dried over Na₂SO₄. The solvent was added Et₃N (10.1 g, 0.1 mol). The resulting suspension was added dropwise Di-tert-butyl pyrocarbonate (15.3 g in 100 ml DCM). After the completion of the reaction, the solvent was removed to give the crude product, which was subjected to flash column chromatography to give pure 2H (14.1 g) as a yellowish solid. LC-MS (ESI) m/z=574.2/576.2 [M-tBu+H]⁺.

### Step 7: tert-butyl(((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)carbamate (intermediate 2)

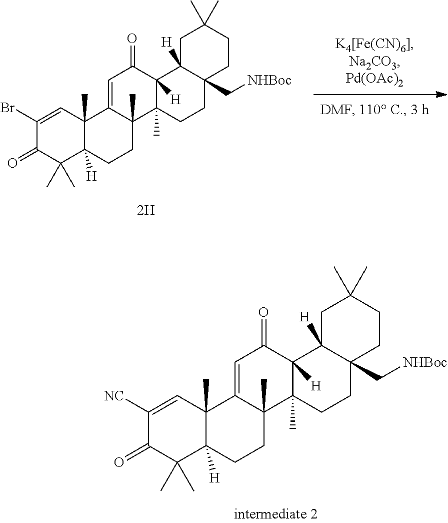

To a solution 2H (0.63 g, 1 mmol) in DMF (20 mL) under argon was successively added K₄[Fe(CN)₆] (0.18 g, 0.5 mmol), sodium Carbonate (0.98 g, 3.0 mmol) and then Pd(OAc)₂ (58 mg, 0.1 mmol). The reaction mixture was heated at 110° C. for 3 h. After cooling to room temperature, the mixture was diluted with water and EtOAc. The organic layer was separated and the aqueous layer extracted with EtOAc. The combined organic layers were then washed with water and brine, dried over Na₂SO₄, filtered, and concentrated in vacuo. The residue was purified by flash chromatography to afford intermediate 2 (0.19 g, 35%) as a yellow solid. LC-MS (ESI) m/z 521.2 [M-tBu+H]⁺. ¹H NMR (400 MHz, CDCl₃) δ 8.03 (s, 1H), 5.97 (s, 1H), 4.62 (s, 1H), 3.28-3.24 (m, 1H), 3.18 (d, J=4.1 Hz, 1H), 3.10-3.04 (m, 1H), 2.23 (s, 1H), 2.04-1.90 (m, 2H), 1.90-1.66 (m, 4H), 1.63-1.57 (m, 2H), 1.56 (s, 3H), 1.55-1.51 (m, 1H), 1.51 (s, 3H), 1.450-1.46 (m, 1H), 1.42 (s, 9H), 1.36-1.30 (m, 1H), 1.29 (s, 2H), 1.28 1.24 (m, 3H), 1.24-1.19 (m, 1H), 1.19-1.04 (m, 2H), 1.14-1.03 (m, 1H), 1.01 (s, 3H), 0.93 (s, 3H), 0.87 (s, 3H).

### Intermediate 3: (4aR,6aS,6bR,8aS,11S,12aS,12bR,14bS)-11-amino-4,4,6a,6b,8a,11,14b-heptamethyl-3,13-dioxo-3,4,4a,5,6,6a,6b,7,8,8a,9,10,11,12,12a,12b,13,14b-octadecahydropicene-2-carbonitrile

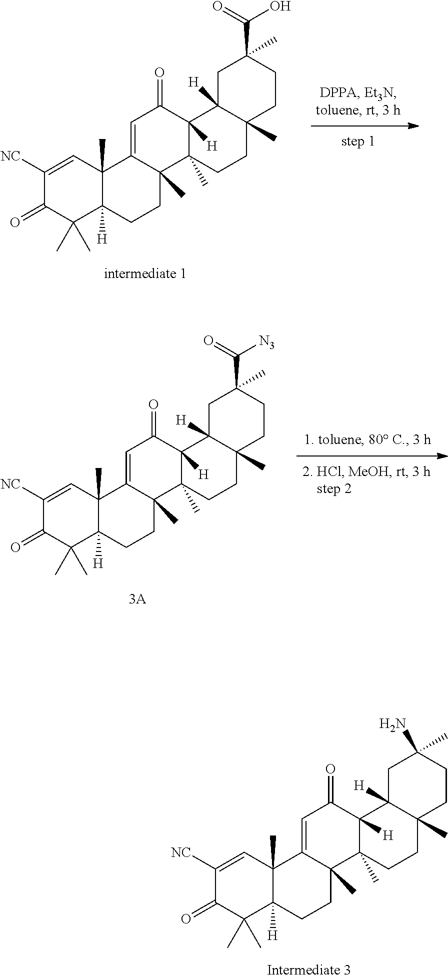

### Step 1: (2S,4aS,6aR,6bS,8aR,12aS,14aR,14bS)-11-cyano-2,4a,6a,6b,9,9,12a-heptamethyl -10,14-dioxo-1,2,3,4,4a,5,6,6a,6b,7,8,8a,9,10,12a,14,14a,14b-octadecahydropicene-2-carbonyl azide (3A)

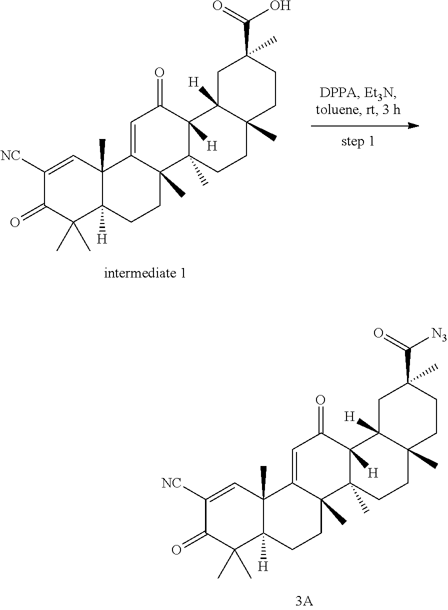

To a stirred solution of intermediate 1 (2.0 g, 4.1 mmol), DPPA (3.0 g, 12 mmol) in toluene (10 mL) at 0° C. was added Et₃N (4.1 g, 41.0 mmol) under nitrogen. The resulting solution was stirred at room temperature for 3 hours. Upon consumption of the starting material, the reaction mixture was diluted with EtOAc and washed with brine (20 mL). The organic layer was dried (Na₂SO₄), filtered and concentrated under reduced pressure. The crude product was purified by flash silica chromatography to afford 3A (1.4 g, 67%) as a white solid.

### Step 2: (4aR,6aS,6bR,8aS,11S,12aS,12bR,14bS)-11-amino-4,4,6a,6b,8a,11,14b-heptamethyl -3,13-dioxo-3,4,4a,5,6,6a,6b,7,8,8a,9,10,11,12,12a,12b,13,14b-octadecahydropicene-2-carbonitrile (intermediate 3)

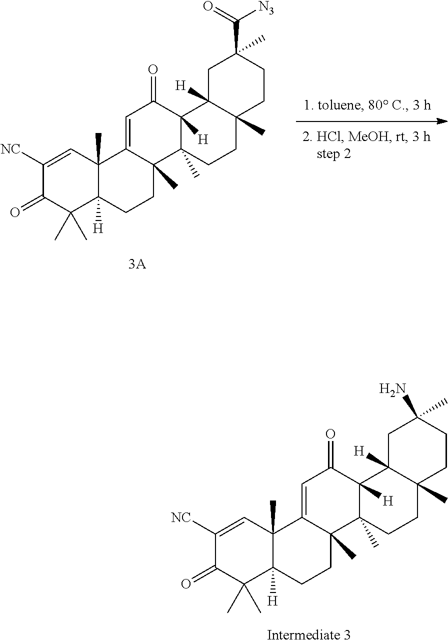

A solution of 3A (1.4 g, 2.7 mmol) in toluene (10 mL) was heated to 80° C. The resulting solution was stirred at 80° C. for 3 h. The reaction mixture was concentrated under reduced pressure. The crude product was dissolved in MeOH (10 mL), and was added concentrated hydrochloric acid (2 mL) at 0° C., The resulting solution was stirred at room temperature for 3 hours. Upon consumption of the starting material, the reaction mixture was diluted with EtOAc (20 mL) and washed with saturated NaHCO₃ (20 mL). The organic layer was dried (Na₂SO₄), filtered and concentrated under reduced pressure. The crude product was purified by flash silica chromatography to afford intermediate 3 (1.2 g, 91%) as a white solid. LC-MS (ESI): m/z=463.3 [M+H]⁺.

### Example 1: (4aR,6aS,6bR,8aS,11S,12aS,12bR,14bS)-4,4,6a,6b,8a,11,14b-heptamethyl-11-(5-methyl-1,3,4-oxadiazol-2-yl)-3,13-dioxo-3,4,4a,5,6,6a,6b,7,8,8a,9,10,11,12,12a,12b,13,14b-octadecahydropicene-2-carbonitrile (compound 1)

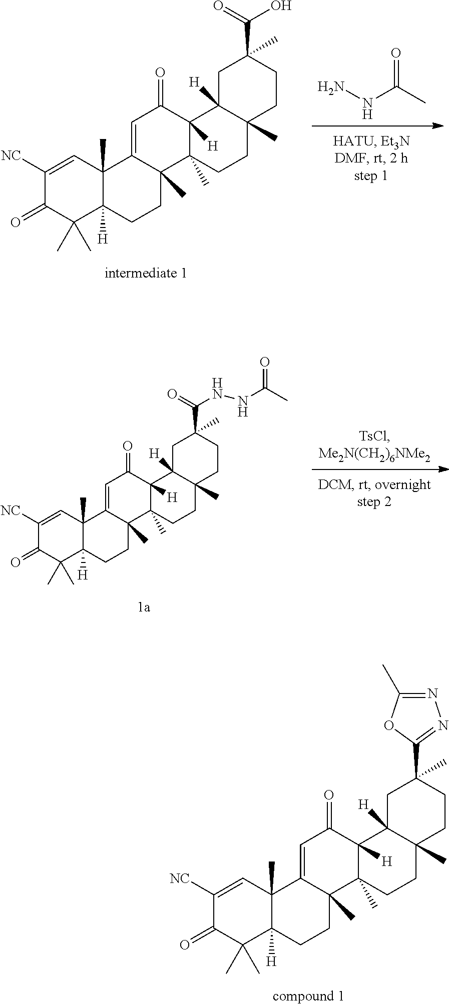

### Step 1: (2S,4aS,6aR,6bS,8aR,12aS,14aR,14bS)-N′-acetyl-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicene-2-carbohydrazide (1a)

To a solution of intermediate 1 (5.5 g, 11 mmol) in DMF (56 mL) was added HATU (5.1 g, 13 mmol), Triethylamine (9.4 mL, 68 mmol) and Acetohydrazide (0.99 g, 13 mmol). The reaction mixture was stirred at room temperature for 2 h, washed with water (150 mL), and extracted with DCM (2×150 mL). The combined organic layers were dried over Na₂SO₄, concentrated in vacuo. The residue was purified by flash chromatography to afford la (5.0 g, 82%) as a yellow powder. LC-MS (ESI): m/z=548.4 [M+H]⁺.

### Step 2: (4aR,6aS,6bR,8aS,11S,12aS,12bR,14bS)-4,4,6a,6b,8a,11,14b-heptamethyl-11-(5-methyl-1,3,4-oxadiazol-2-yl)-3,13-dioxo-3,4,4a,5,6,6a,6b,7,8,8a,9,10,11,12,12a,12b,13,14b-octadecahydropicene-2-carbonitrile (compound 1)

To a solution of la (5 g, 9.1 mmol) in DCM (45 mL) was added N¹,N¹,N⁶,N⁶-tetramethylhexane-1,6-diamine (3.1 g, 18.3 mmol) and TsCl (3.5 g, 18.3 mmol). The reaction mixture was stirred at room temperature overnight, washed with water (50 mL), and extracted with EA (2×50 mL). The combined organic layers were dried over Na₂SO₄, concentrated in vacuo. The residue was purified by chromatography to afford compound 1 (3 g, 62%) as a white solid. LC-MS (ESI): m/z=530.3 [M+H]⁺. ¹H NMR (400 MHz, CDCl₃) δ 8.02 (s, 1H), 5.99 (s, 1H), 3.04 (d, 1H), 2.59 (s, 3H), 2.32-2.20 (m, 2H), 2.01-1.68 (m, 10H), 1.62-1.53 (m, 2H), 1.50 (s, 3H), 1.47 (s, 3H), 1.42-1.34 (m, 1H), 1.27 (s, 6H), 1.18 (s, 3H), 1.17-1.09 (m, 1H), 1.02 (s, 3H), 0.92 (s, 3H).

### Example 2: (2S,4aS,6aR,6bS,8aR,12aS,14aR,14bS)-2,4a,6a,6b,9,9,12a-heptamethyl-10,14-dioxo-1,2,3,4,4a,5,6,6a,6b,7,8,8a,9,10,12a,14,14a,14b-octadecahydropicene-2,11-dicarbonitrile (compound 2)

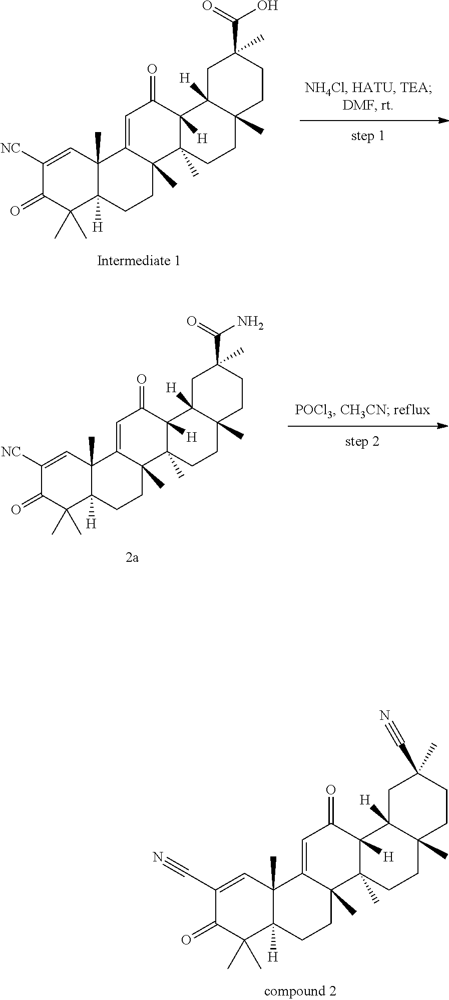

### Step 1: (2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicene-2-carboxamide (2a)

To a solution of intermediate 1 (0.160 g, 0.325 mmol), HATU (0.148 g, 0.390 mmol) and TEA (0.066 g, 0.65 mmol) in DCM (5 mL) was added NH₄Cl (0.052 g, 0.976 mmol) at 25° C. The reaction mixture was stirred for 2 h, washed with water (20 mL), and extracted with DCM (2×10 mL). The combined organic layers were dried over Na₂SO₄, concentrated in vacuo. The residue was used to next step directly.

LC-MS (ESI): m/z=491.4 [M+H]⁺.

### Step 2: (2S,4aS,6aR,6bS,8aR,12aS,14aR,14bS)-2,4a,6a,6b,9,9,12a-heptamethyl- 10,14-dioxo-1,2,3,4,4a,5,6,6a,6b,7,8,8a,9,10,12a,14,14a,14b-octadecahydropicene-2,11-dicarbonitrile (compound 2)

To a solution of 2a (0.12 g, 0.245 mmol) in MeCN (5 mL) was added POCl₃ (0.375 g, 2.45 mmol) at 0° C. The reaction mixture was warmed to room temperature and then refluxed for 2 h, washed with water (20 mL), and extracted with DCM (2×10 mL). The combined organic layers were dried over Na₂SO₄, concentrated in vacuo. The residue was purified by flash chromatography to afford compound 2 (0.034 g, 34.6%) as a white solid. LC-MS (ESI): m/z=473.4 [M+H]⁺.

¹H NMR (400 MHz, CDCl₃) δ 8.04 (s, 1H), 6.04 (s, 1H), 3.17 (d, 1H), 2.41 (d, 1H), 2.12-2.04 (m, 1H), 1.95-1.66 (m, 8H), 1.60 (d, 1H), 1.52 (s, 5H), 1.51-1.48 (m, 1H), 1.46 (d, 1H), 1.42 (d, 1H), 1.32 (d, 4H), 1.26 (s, 3H), 1.18 (s, 3H), 1.16 1.09 (m, 1H), 1.06 (s, 3H), 1.00 (d, 1H), 0.96 (s, 3H).

### Example 3 : 2-cyano-1-(((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-3-methylguanidine (compound 3)

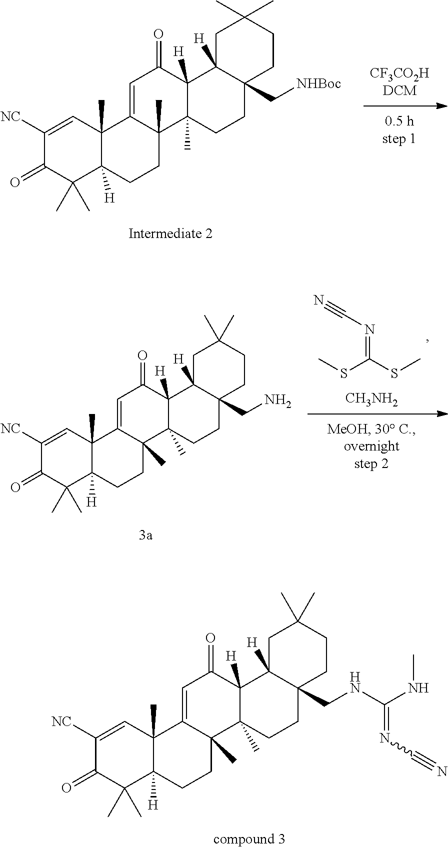

### Step 1: (4aR,6aS,6bR,8aS,12aS,12bR,14bS)-8a-(aminomethyl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile (3a)

A solution of intermediate 2 (0.15 g, 0.26 mmol) and trifluoroacetic acid (3 mL) in DCM (10 mL) was stirred at room temperature for 0.5 h. Solvent was evaporated, and the crude product was partitioned between water and DCM. The aqueous layer was basified with NaHCO₃ and extracted with DCM. Combined organic layers were washed with brine, dried over sodium sulfate, filtered, and evaporated to give 3a (0.12 g, 100%) that was used in the next step without further purification. LC-MS (ESI) m/z 477.3 [M+H]⁺.

### Step 2: 2-cyano-1-(((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)-3-methylguanidine (compound 3)

To a solution of 3a (0.10 g, 0.21 mmol) in MeOH (10 mL) was added bis(methylsulfanyl)methylenecyanamide (44 mg, 0.30 mmol), and stirred overnight at 30° C. Then methanamine in EtOH (1.0 mL, 2.0 mmol) was added to the reaction mixture. After another 20 h, the reaction mixture was concentrated in vacuo. The residue was purified by chromatography on silica gel (PE: EA=1/1 to 1/2) to afford compound 3 (20 mg, 20%) as a pale yellow solid. LC-MS (ESI): m/z=558.4 [M+H]⁺.

### Example 4: N′-cyano-N-(((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)methyl)acetimidamide (compound 4)

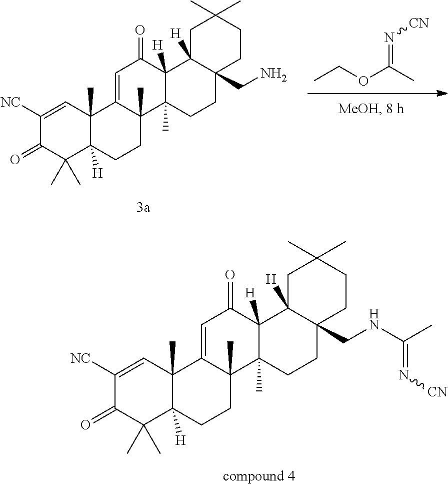

To a solution of 3a (0.12 g, 0.26 mmol) in MeOH (5 mL) was added ethyl N-cyanoacetimidate (0.5 mL). The reaction mixture was stirred for 8 h. After the completion of the reaction, the mixture was concentrated in vacuo. The residue was purified by flash chromatography to afford Compound 4 (35 mg) as a yellow solid. LC-MS (ESI): m/z 543.4 [M+H]⁺. ¹H NMR (400 MHz, CDCl₃) δ 8.05 (s, 1H), 6.01-5.96 (m, 2H), 3.75-3.70 (m, 1H), 3.23-3.18 (m, 1H), 3.10 (d, J=4.6 Hz, 1H), 2.37 (s, 3H), 2.26-2.22 (m, 1H), 2.08-1.67 (m, 6H), 1.63 (s, 3H), 1.60-1.56 (m, 1H), 1.55 (s, 3H), 1.51 (s, 3H), 1.46-1.40 (m, 1H), 1.35-1.28 (m, 2H), 1.27 (s, 3H), 1.19 (s, 3H), 1.14-1.08 (m, 1H), 1.01 (s, 3H), 0.98-0.93 (m, 1H)0.92 (s, 3H), 0.89 (s, 3H).

### Example 5: N-((4aS,6aR,6bS,8aR,12aS,14aR,14bS)-11-cyano-2,2,6a,6b,9,9,12a-heptamethyl- 10,14-dioxo-1,3,4,5,6,6a,6b,7,8,8a,9,10,12a,14,14a,14b-hexadecahydropicen-4a(2H)-yl)-2-((E)-N-cyano-S-methylsulfinimidoyl)acetamide (compound 5)

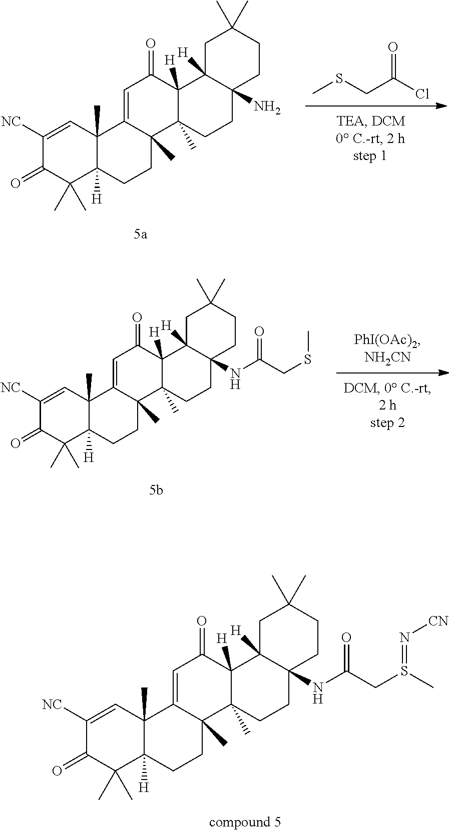

### Step 1: N-((4aS,6aR,6bS,8aR,12aS,14aR,14bS)-11-cyano-2,2,6a,6b,9,9,12a-heptamethyl -10,14 -dioxo-1,3,4,5,6,6a,6b,7,8,8a,9,10,12a,14,14a,14b-hexadecahydropicen-4a(2H)-yl)-2-(methylthio)acetamide (5b)

To a solution of 5a (0.30 g, 0.65 mmol) and TEA (0.20 g, 1.9 mmol) in DCM (5 mL) was added 2-(methylthio)acetyl chloride (0.12 g, 0.97 mmol) at 0° C. The reaction mixture was warmed to room temperature and stirred for 2 h, washed with water (20 mL), and extracted with DCM (2×10 mL). The combined organic layers were dried over Na₂SO₄, concentrated in vacuo. The residue was purified by flash chromatography to afford 5b (0.21 g, 59%) as a white solid. LC-MS (ESI): m/z=551.3 [M+H]⁺.

### Step 2: N-((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-2-((E)-N- cyano-S-methylsulfinimidoyl)acetamide (compound 5)

To a solution of 5a (0.17 g, 0.31 mmol) and NH₂CN (0.20 g, 1.9 mmol) in DCM (3 mL) was added PhI(OAc)₂ (0.10 g, 0.34 mmol) at 0° C. The reaction mixture was warmed to room temperature and stirred for 2 h, washed with water (20 mL), and extracted with DCM (2×10 mL). The combined organic layers were dried over Na₂SO₄, concentrated in vacuo. The residue was purified by flash chromatography to afford compound 5 (0.11 g, 60%) as a white solid. LC-MS (ESI): m/z=591.3 [M+H]⁺.¹H NMR (400 MHz, CDCl₃) δ 8.64 (s, 1H), 7.97 (d, 1H), 6.23 (s, 1H), 4.11 (d, 1H), 4.00 (t, 1H), 3.11 (s, 1H), 2.83-2.50 (m, 4H), 2.07-0.94 (m, 33H), 0.86 (s, 3H).

### Example 6: (4aR,6aS,6bR,8aS,11S,12aS,12bR,14bS)-4,4,6a,6b,8a,11,14b-heptamethyl-11-(oxazol-2-yl)-3,13-dioxo-3,4,4a,5,6,6a,6b,7,8,8a,9,10,11,12,12a,12b,13,14b-octadecahydropicene-2-carbonitrile (compound 6)

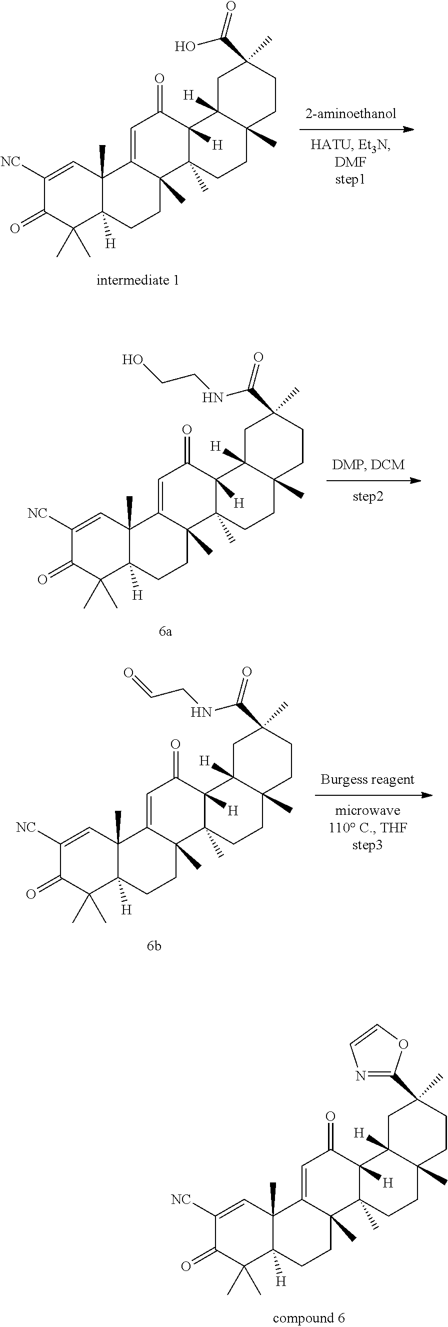

### Step 1: (2S,4aS,6aR,6bS,8aR,12aS,14aR,14bS)-11-cyano-N-(2-hydroxyethyl)-2,4a,6a,6b,9,9, 12a- heptamethyl-10,14-dioxo-1,2,3,4,4a,5,6,6a,6b,7,8,8a,9,10,12a,14,14a,14b-octadecahydropicene-2-carboxamide (6a)

To a solution of intermediate 1 (0.12 g, 0.24 mmol) in DMF (500 mL) were successively added HATU (0.15 g, 0.36 mmol) and Et₃N (41 mg, 0.4 mmol). The reaction mixture was stirred at room temperature for 0.5 h, and the mixture was added 2-aminoethanol (60 mg, 1 mmol) and stirred at room temperature for 3 h. The mixture was diluted with water and EtOAc. The organic layer was separated and the aqueous layer extracted with EtOAc. The combined organic layers were then washed with water and brine, dried over Na₂SO₄, filtered and concentrated in vacuo. The residue was purified by flash chromatography to afford 6a (0.11 g, 84%) as a yellow solid. LC-MS (ESI): m/z 535.4 [M+H]⁺

### Step 2: (2S,4aS,6aR,6bS,8aR,12aS,14aR,14bS)-11-cyano-2,4a,6a,6b,9,9,12a-heptamethyl-10,14-dioxo-N-(2-oxoethyl)-1,2,3,4,4a,5,6,6a,6b,7,8,8a,9,10,12a,14,14a,14b-octadecahydropicene-2-carboxamide (6b)

A solution of 6a (0.11 g, 0.2 mmol) in DCM (10 mL) was added DMP (0.42 g, 1 mmol) and stirred at room temperature for 1 h. The mixture was quenched with aqueous NaHCO₃ and extracted with DCM. Combined organic layers were washed with brine, dried over sodium sulfate, filtered, and evaporated to give 6b (90 mg, 82%) that was used in the next step without further purification. LC-MS (ESI) m/z 533.4 [M+H]³⁰

### Step 3: (4aR,6aS,6bR,8aS,11S,12aS,12bR,14bS)-4,4,6a,6b,8a,11,14b-heptamethyl-11-(oxazol -2-yl)-3,13-dioxo-3,4,4a,5,6,6a,6b,7,8,8a,9,10,11,12,12a,12b,13,14b-octadecahydropicene-2-carbonitrile (compound 6)

A solution of 6b (90 mg, 0.17 mmol) in THF (10 mL) was added Burgess reagent (0.13 g, 0.51 mmol), the mixture was stirred in microwave at 110° C. for 1 h. Then the mixture was concentrated in vacuo. The residue was purified by flash chromatography to afford Compound 6 (32 mg, 37%) as a yellow solid. LC-MS (ESI): m/z 515.4 [M+H]⁺. ¹H NMR (400 MHz, CDCl₃) δ 8.04 (s, 1H), 7.72 (s, 1H), 7.15 (s, 1H), 6.00 (s, 1H), 3.05 (d, J=4.7 Hz, 1H), 2.49-2.21 (m, 3H), 2.02-1.71 (m, 7H), 1.65-1.52 (m, 3H), 1.50 (s, 3H), 1.46 (s, 3H), 1.45-1.35(m, 1H), 1.27 (s, 3H), 1.26 (s, 3H), 1.18 (s, 3H), 1.16-1.09 (m, 1H), 1.04 (s, 3H), 1.03-0.94 (m, 1H), 0.90 (s, 3H).

### Example 7: (4aR,6aS,6bR,8aS,11S,12aS,12bR,14bS)-4,4,6a,6b,8a,11,14b-heptamethyl-11-(5-methyloxazol-2-yl)-3,13-dioxo-3,4,4a,5,6,6a,6b,7,8,8a,9,10,11,12,12a,12b,13,14b-octadecahydropicene-2-carbonitrile (compound 7)

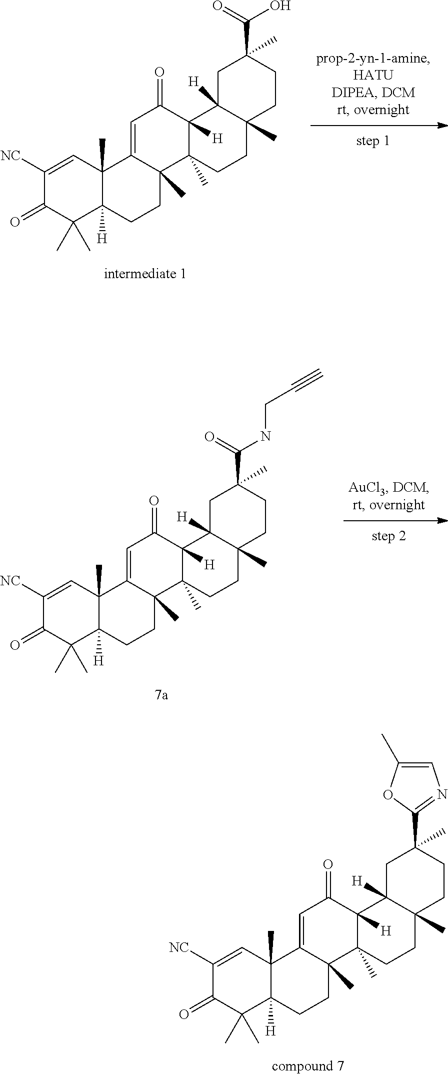

### Step 1: (2S,4aS,6aR,6bS,8aR,12aS,14aR,14bS)-11-cyano-2,4a,6a,6b,9,9,12a-heptamethyl-10,14-dioxo-N- (prop-2-yn-1-yl)-1,2,3,4,4a,5,6,6a,6b,7,8,8a,9,10,12a,14,14a,14b-octadecahydropicene-2-carboxamide (7a)

To a stirred solution of intermediate 1 (0.50 g,1.00 mmol), HATU(0.58g, 1.50 mmol) and prop-2-yn-1-amine (0.07 g, 1.2 mmol) in DCM (10 mL) at room temperature was added DIPEA (0.4 g, 3.0 mmol) under nitrogen. The resulting solution was stirred at room temperature overnight. Upon consumption of the starting material, the reaction mixture was diluted with EtOAc and washed with brine (20 mL). The organic layer was dried (Na₂SO₄), filtered and concentrated under reduced pressure. The crude product was purified by flash silica chromatography to afford 7a (0.48 g, 89%) as a white solid. LC-MS (ESI): m/z =529.3 [M+H]⁺.

### Step 2: (4aR,6aS,6bR,8aS,11S,12aS,12bR,14bS)-4,4,6a,6b,8a,11,14b-heptamethyl-11-(5-methyl oxazol-2-yl)-3,13-dioxo-3,4,4a,5,6,6a,6b,7,8,8a,9,10,11,12,12a,12b,13,14b-octadecahydropicene-2-carbonitrile (compound 7)

To a solution of 7a (0.43 g, 0.81 mmol) in DCM (10 mL) was added AuCl₃ (49 mg, 0.16 mmol) under nitrogen. The resulting solution was stirred at room temperature overnight and concentrated under reduced pressure. The crude product was purified by flash silica chromatography to afford compound 7 (114 mg, 26%) as a white solid. LC-MS (ESI): m/z=529.4 [M+H]⁺. ¹H NMR (400 MHz, CDCl₃) δ 8.02 (s, 1H), 7.00 (d, 1H), 6.03 (s, 1H), 3.05 (d, 1H), 2.43 (m, 4H), 2.27 (m, 1H), 2.04-1.67 (m, 7H), 1.66-1.36 (m, 11H), 1.28 (m, 6H), 1.24 1.10 (m, 4H), 1.04 (m, 4H), 0.91 (s, 3H).

### Example 8: (4aR,6aS,6bR,8aS,11S,12aS,12bR,14bS)-4,4,6a,6b,8a,11,14b-heptamethyl-3,13-dioxo-11-(3-phenyl-1H-1,2,4-triazol-5-yl)-3,4,4a,5,6,6a,6b,7,8,8a,9,10,11,12,12a,12b,13,14b-octadecahydropicene-2carbonitrile (compound 8)

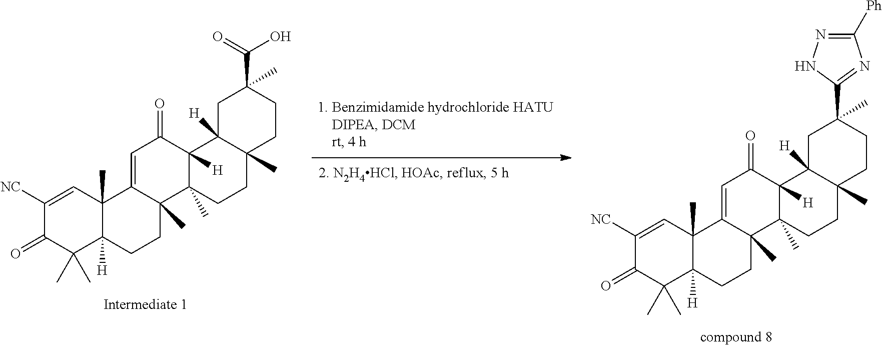

To a stirred solution of intermediate 1 (50 mg,0.10 mmol), HATU (50 mg, 0.13 mmol) and benzimidamide hydrochloride (21 mg, 0.13 mmol) in DCM (4 mL) at room temperature was added DIPEA (53 mg, 0.41mmol) under nitrogen. The resulting solution was stirred at room temperature for 4 hours. Upon consumption of the starting material, hydrazine hydrochloride (56 mg, 0.81 mmol) and acetic acid (12 mg, 0.20 mmol) were added to the reaction mixture that heated to reflux for 5 h. After consumption of intermediate, the reaction mixture was diluted with EtOAc and washed with saturated NaHCO₃ (20 mL). The organic layer was dried (MgSO₄), filtered and concentrated under reduced pressure. The crude product was purified by flash silica chromatography to afford compound 8 (11.7 mg, 20%) as a white solid. LC-MS (ESI): m/z=591.3 [M+H]⁺. ¹H NMR (400 MHz, CDCl₃) δ 8.15 (d, 2H), 8.07 (s, 1H), 7.49-7.33 (m, 3H), 6.12 (s, 1H), 3.12 (d, 1H), 2.23 (s, 1H), 2.05 (m, 3H), 1.89 (d, 2H), 1.83 (s, 3H), 1.68-1.60 (m, 3H), 1.55 (s, 3H), 1.52 (s, 3H), 1.42-1.36 (m, 4H), 1.27 (m, 4H), 1.23-1.11 (m, 6H), 1.05 (s, 3H), 0.90 (s, 3H).

### Example 9: (4aR,6aS,6bR,8aS,11S,12aS,12bR,14bS)-4,4,6a,6b,8a,11,14b-heptamethyl-11-(1,3,4-oxadiazol-2-yl)-3,13-dioxo-3,4,4a,5,6,6a,6b,7,8,8a,9,10,11,12,12a,12b,13,14b-octadecahydropicene-2-carbonitrile (compound 9)

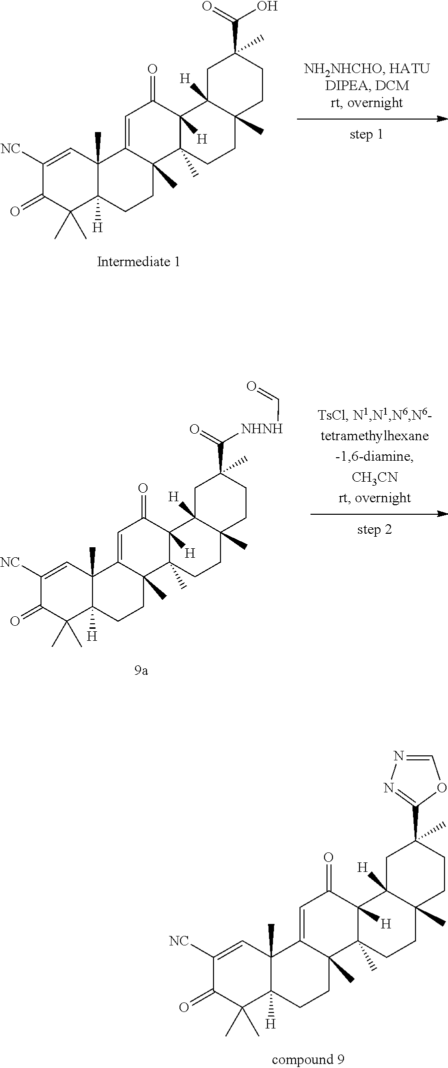

### Step 1: (2S,4aS,6aR,6bS,8aR,12aS,14aR,14bS)-11-cyano-AP-formyl-2,4a,6a,6b,9,9,12a-hepta methyl-10,14-dioxo-1,2,3,4,4a,5,6,6a,6b,7,8,8a,9,10,12a,14,14a,14b-octadecahydropicene-2-carbohydrazide (9a)

To a stirred solution of intermediate 1(1.30 g, 2.60 mmol), HATU(1.20 g, 3.20 mmol) and formylhydrazine (0.21 g, 3.40 mmol) in DCM (10 mL) at room temperature was added DIPEA (1.0 g, 7.91 mmol) under nitrogen. The resulting solution was stirred at room temperature overnight. Upon consumption of the starting material, the reaction mixture was diluted with EtOAc and washed o with brine (20 mL). The organic layer was dried (MgSO₄), filtered and concentrated under reduced pressure. The crude product was purified by flash silica chromatography to afford 9a (1.1 g, 78%) as a white solid. LC-MS (ESI): m/z=534.3 [M+H]⁺.

### Step 2: (4aR,6aS,6bR,8aS,11S,12aS,12bR,14bS)-4,4,6a,6b,8a,11,14b-heptamethyl-11-(1,3,4-oxadiazol-2-yl)-3,13-dioxo-3,4,4a,5,6,6a,6b,7,8,8a,9,10,11,12,12a,12b,13,14b-octadecahydropicene-2-carbonitrile (compound 9)

To a solution of 9a (1.10 g, 2.06 mmol) in DCM (5 mL) was added TsCl (0.79 g, 4.12 mmol) and N¹,N¹,N⁶,N⁶-tetramethylhexane-1,6-diamine (0.98 g, 6.18 mmol). The reaction mixture was stirred at room temperature overnight, the reaction mixture was diluted with EtOAc and washed with saturated NH₄Cl solution (20 mL). The organic layer was dried (Na₂SO₄), filtered and concentrated under reduced pressure. The crude product was purified by flash silica chromatography to afford compound 9 (170 mg, 16%) as a white solid. LC-MS (ESI): m/z=516.3 [M+H]⁺. ¹H NMR (400 MHz, CDCl₃) δ 8.44 (s, 1H), 8.03 (s, 1H), 5.99 (s, 1H), 3.04 (d, 1H), 2.33 (m, 2H), 2.02 1.76 (m, 7H), 1.71 1.53 (m, 6H), 1.49 (d, 5H), 1.40 (d, 1H), 1.28 (d, 6H), 1.21 1.09 (m, 4H), 1.02 (d, 3H), 0.91 (s, 3H).

### Example 10: (4aR,6aS,6bR,8aS,11S,12aS,12bR,14bS)-4,4,6a,6b,8a,11,14b-heptamethyl-3,13-dioxo-11-(5-(trifluoromethyl)-1,3,4-oxadiazol-2-yl)-3,4,4a,5,6,6a,6b,7,8,8a,9,10,11,12,12a,12b,13,14b-octadecahydropicene-2-carbonitrile (compound 10)

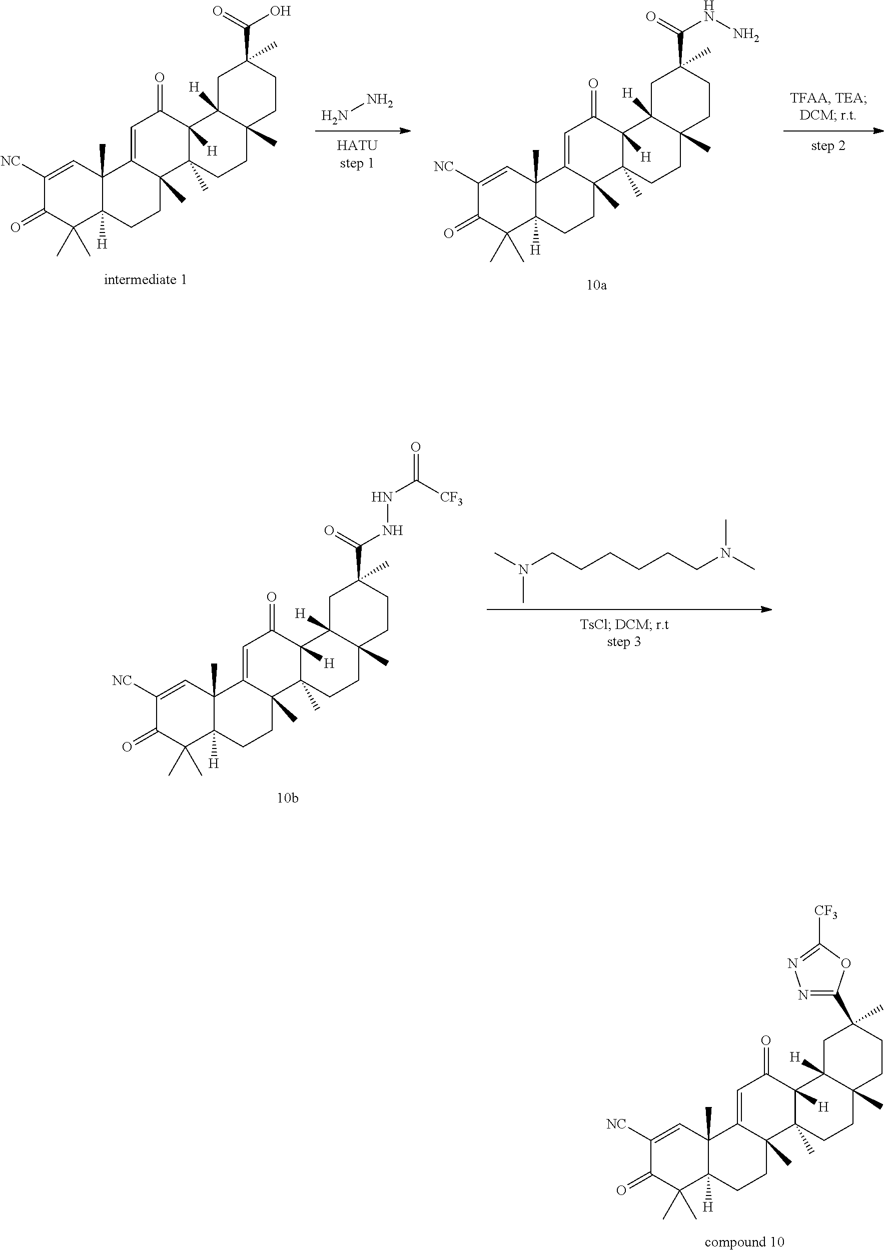

### Step 1: (2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicene-2-carbohydrazide (10a)

To a solution of intermediate 1 (0.3 g, 0.56 mmol) in 1,4-dioxane (10 mL) was added concentrated hydrochloric acid (1 mL) . The resulting solution was stirred at room temperature overnight. Upon consumption of the starting material, the reaction mixture was diluted with EtOAc (20 mL) and washed with saturated NaHCO₃ solution (20 mL). The organic layer was dried (Na₂SO₄), filtered and concentrated under reduced pressure. The crude product was purified by flash silica chromatography to afford 10a (0.2 g, 67%) as a white solid. LC-MS (ESI): m/z=506.3 [M+H]⁺.

### Step 2: (2S,4aS,6aR,6bS,8aR,12aS,14aR,14bS)-11-cyano-2,4a,6a,6b,9,9,12a-heptamethyl-10,14-dioxo-N-(2,2,2-trifluoroacetyl)-1,2,3,4,4a,5,6,6a,6b,7,8,8a,9,10,12a,14,14a,14b-octadecahydropicene-2-carbohydrazide (10b)

To a solution of 10a (0.50 g, 1.0 mmol) and TEA (0.300 g, 3.0 mmol) in DCM (25 mL) was added TFAA (0.310 g, 1.5 mmol) at 25° C. The reaction mixture was stirred for 2 h, washed with water (20 mL), and extracted with DCM (2×10 mL). The combined organic layers were dried over Na₂SO₄, concentrated in vacuo. The residue was used to next step directly. LC-MS (ESI): m/z=602.3 [M+H]⁺.

### Step 3: (4aR,6aS,6bR,8aS,11S,12aS,12bR,14bS)-4,4,6a,6b,8a,11,14b-heptamethyl-3,13-dioxo-11-(5- (trifluoromethyl)-1,3,4-oxadiazol-2-yl)-3,4,4a,5,6,6a,6b,7,8,8a,9,10,11,12,12a,12b,13,14b-octadecahydropicene-2-carbonitrile (compound 10)

To a solution of 10b (0.200 g, 0.332 mmol) and N¹,N¹,N⁶,N⁶-tetramethylhexane-1,6-diamine (0.115 g, 0.665 mmol) in DCM (10 mL) was added TsCl (0.127 g, 0.665 mmol) at 25° C. The reaction mixture was stirred at 25° C. for 16 h, washed with water (20 mL), and extracted with DCM (2×10 mL). The combined organic layers were dried over Na₂SO₄, concentrated in vacuo. The residue was purified by flash chromatography to afford compound 10 (0.040 g, 20.6%) as a white solid. LC-MS (ESI): m/z=584.4 [M+H]⁺. ¹H NMR (400 MHz, CDCl₃) δ 8.02 (s, 1H), 6.03 (s, 1H), 3.05 (d, 1H), 2.32 (d, 2H), 1.78-1.75 (m, 6H), 1.68-1.56 (m, 3H), 1.55-1.39 (m, 7H), 1.34 (s, 3H), 1.27 (s, 5H), 1.20-1.11 (m, 4H), 1.04 (d, 4H), 0.93 (s, 3H).

### Example 11: (4aR,6aS,6bR,8aS,11S,12aS,12bR,14bS)-4,4,6a,6b,8a,11,14b-heptamethyl-11-morpholino-3,13-dioxo-3,4,4a,5,6,6a,6b,7,8,8a,9,10,11,12,12a,12b,13,14b-octadecahydropicene-2-carbonitrile (compound 11)

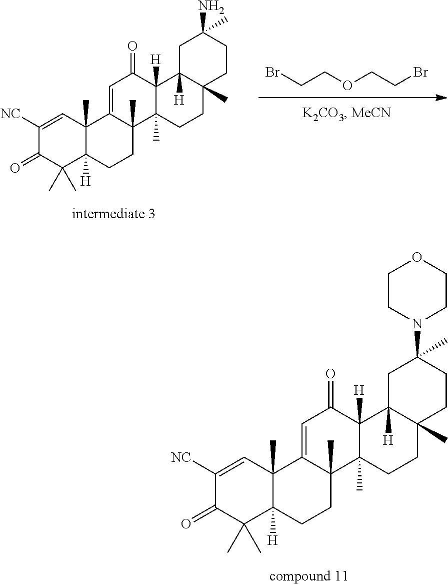

To a solution of intermediate 3 (0.100 g, 0.216 mmol) and K₂CO₃ (0.090 g, 0.648 mmol) in MeCN (5 mL) was added 1-bromo-2-(2-bromoethoxy)ethane (0.060 g, 0.259 mmol) at 25° C. The reaction mixture was stirred at 90° C. for 4 h, washed with water (20 mL), and extracted with DCM (2×10 mL). The combined organic layers were dried over Na₂SO₄, concentrated in vacuo. The residue was purified by flash chromatography to afford compound 11 (0.040 g, 34.7%) as a white solid. LC-MS (ESI): m/z=533.5 [M+H]⁺. ¹H NMR (400 MHz, CDCl₃) δ 8.02 (s, 1H), 5.95 (s, 1H), 3.73 (s, 4H), 3.06 (d, 1H), 2.44-2.42 (m, 3H), 2.03-1.92 (m, 1H), 1.92-1.85 (m, 1H), 1.85-1.75 (m, 6H), 1.75-1.55 (m, 4H), 1.49 (d, 6H), 1.28-1.23 (m, 4H), 1.21-1.15 (m, 4H), 1.13 (s, 1H), 1.11-1.03 (m, 1H), 0.98 (t, 6H), 0.92-0.90 (m, 1H), 0.80 (s, 3H).

### Example 12: (4aR,6aS,6bR,8aS,11S,12aS,12bR,14bS)-4,4,6a,6b,8a,11,14b-heptamethyl-3,13-dioxo-11-(2-oxopyrrolidin-1-yl)-3,4,4a,5,6,6a,6b,7,8,8a,9,10,11,12,12a,12b,13,14b-octadecahydropicene-2-carbonitrile (compound 12)

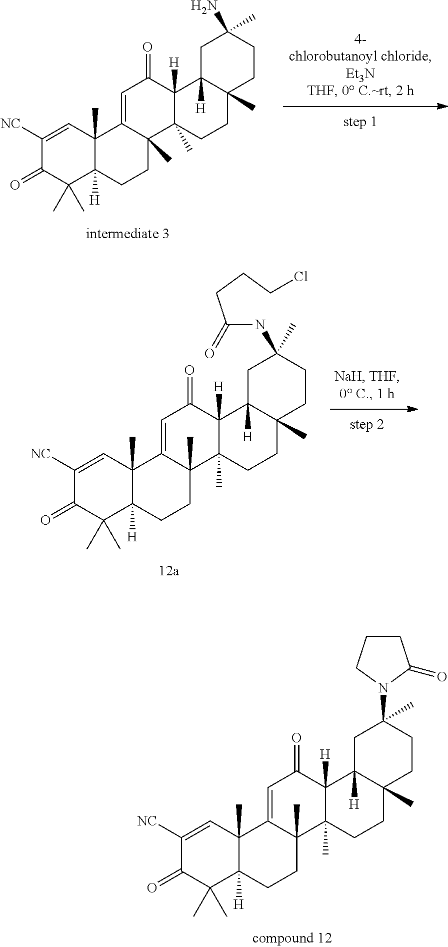

### Step 1: 4-chloro-N-((2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicen-2-yl)butanamide (12a)

To a stirred solution of intermediate 3 (0.18 g, 0.18 mmol) in THF (5 mL) at 0° C. was added Et₃N (0.2 g, 1.9 mmol), 4-chlorobutanoyl chloride (0.11 g, 0.78 mmol) under nitrogen. The resulting solution was stirred at room temperature for 2 hours. Upon consumption of the starting material, the reaction mixture was diluted with EtOAc and washed with brine (20 mL). The organic layer was dried (Na₂SO₄), filtered and concentrated under reduced pressure. The crude product was purified by flash silica chromatography to afford 12a (0.1 g, 45%) as a white solid. LC-MS (ESI): m/z=567.3[M+H]⁺.

### Step 2: (4aR,6aS,6bR,8aS,11S,12aS,12bR,14bS)-4,4,6a,6b,8a,11,14b-heptamethyl-3,13-dioxo- 11-(2- oxopyrrolidin-1-yl)-3,4,4a,5,6,6a,6b,7,8,8a,9,10,11,12,12a,12b,13,14b-octadecahydropicene-2-carbonitrile (compound 12)

To a stirred solution of 12a (0.10 g, 0.18 mmol) in THF (5 mL) at 0° C. was added (50% w/w) NaH (21 mg, 0.88 mmol) under nitrogen. The resulting solution was stirred at room temperature for 1 hour. The reaction mixture was diluted with EtOAc and washed with brine (10 mL). The organic layer was dried over Na₂SO₄, filtered and concentrated under reduced pressure. The crude product was purified by flash silica chromatography to afford compound 12 (24 mg, 26%) as a white solid. LC-MS (ESI): m/z=531.4 [M+H]⁺. ¹H NMR (400 MHz, CDCl₃) δ 8.03 (s, 1H), 5.98 (s, 1H), 4.06 (m, 1H), 3.28 (m, 2H), 3.11 (d, 1H), 2.49-2.23 (m, 2H), 2.05-1.77 (m, 10H), 1.67 (s, 3H), 1.58 (m, 2H), 1.50 (d, 6H), 1.39-1.23 (m, 8H), 1.20 (d, 3H), 1.14 (s, 3H), 1.03-0.94 (m, 3H).

### Example 13: (4aR,6aS,6bR,8aS,11S,12aS,12bR,14bS)-4,4,6a,6b,8a,11,14b-heptamethyl-3,13 -dioxo-11-(2-oxooxazolidin-3-yl)-3,4,4a,5,6,6a,6b,7,8,8a,9,10,11,12,12a,12b,13,14b-octadecahydropicene-2-carbonitrile (compound 13)

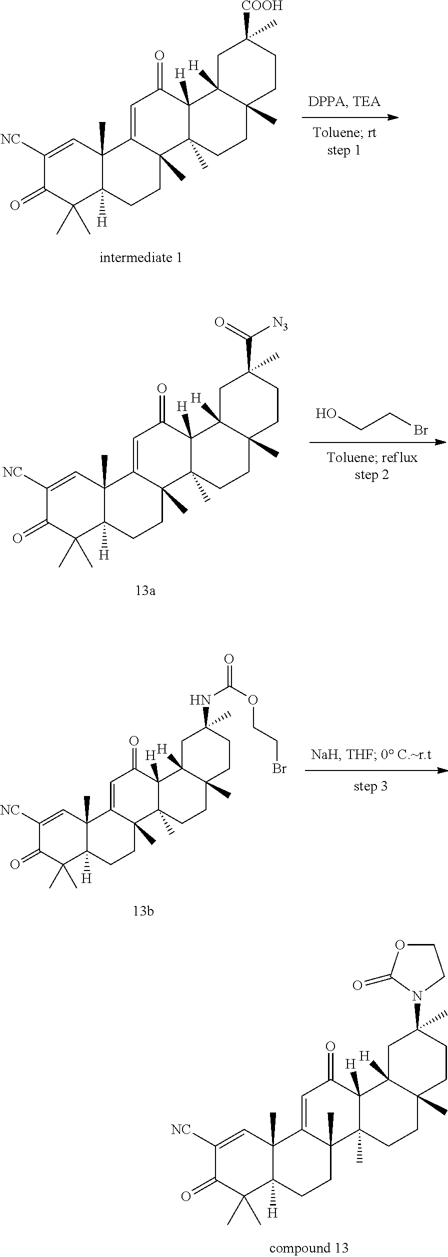

### Step 1: (2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicene-2-carbonyl azide (13a)

To a solution of intermediate 1 (1.0 g, 2.0 mmol) and TEA (0.62 g, 6.0 mmol) in Toluene (20 mL) was added DPPA (0.84 g, 0.259 mmol) at 25° C. The reaction mixture was stirred at 25° C. for 2 h, washed with water (20 mL), and extracted with EA (2×20 mL). The combined organic layers were dried over Na₂SO₄, concentrated in vacuo. The residue was purified by flash chromatography to afford compound 13a (0.80 g, 76%) as a white solid.

### Step 2: 2-bromoethyl ((2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicen-2-yl)carbamate (13b)

To a solution of 13a (0.220 g, 0.426 mmol) in Toluene (20 mL) was added 2-bromoethan-1-ol (0.213 g, 1.70 mmol) at 25° C. The reaction mixture was stirred at 110° C. for 2 h, washed with water (20 mL), and extracted with EA (2×20 mL). The combined organic layers were dried over Na₂SO₄, concentrated in vacuo. The residue was purified by flash chromatography to afford compound 13b (0.120 g, 46%) as a white solid. LC-MS (ESI): m/z=613.3 [M+H]³⁰ .

### Step 3: (4aR,6aS,6bR,8aS,11S,12aS,12bR,14bS)-4,4,6a,6b,8a,11,14b-heptamethyl-3,13-dioxo -11-(2- oxooxazolidin-3-yl)-3,4,4a,5,6,6a,6b,7,8,8a,9,10,11,12,12a,12b,13,14b-octadecahydropicene-2-carbonitrile (compound 13)

To a solution of 13b (0.070 g, 0.11 mmol) in THF (5 mL) was added NaH (0.02 g, 0.46 mmol) at 0° C. The reaction mixture was stirred at 25° C. for 2 h, washed with water (20 mL), and extracted with EA (2×20 mL). The combined organic layers were dried over Na₂SO₄, concentrated in vacuo. The residue was purified by flash chromatography to afford compound 13 (0.025 g, 41%) as a white solid. LC-MS (ESI): m/z=533.4 [M+H]⁺. ¹H NMR (400 MHz, CDCl₃) δ 8.02 (s, 1H), 5.97 (s, 1H), 4.35-4.14 (m, 3H), 3.58-3.48 (m, 1H), 3.12 (d, 1H), 3.05-3.01 (m, 1H), 2.07 2.00 (m, 1H), 1.98-1.87 (m, 2H), 1.87-1.71 (m, 5H), 1.66-1.64 (m, 2H), 1.55 (s, 3H), 1.51 (s, 3H), 1.40-1.24 (m, 9H), 1.21-1.10 (m, 7H), 1.00 (m, 7H).

### Example 14 : (4aR,6aS,6bR,8aR,11S,12aS,12bR,14bS)-11-((1,3,4-oxadiazol-2-yl)methyl)-4,4,6a,6b,8a,11,14b-heptamethyl-3,13-dioxo-3,4,4a,5,6,6a,6b,7,8,8a,9,10,11,12,12a,12b,13,14b-octadecahydropicene-2-carbonitrile (compound 14)

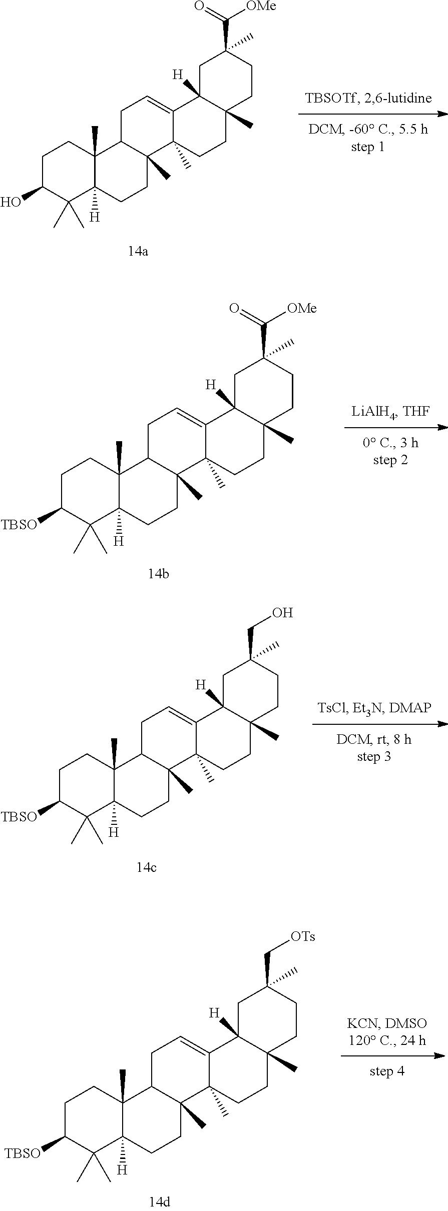 [SMILES: COC(=O)[C@@]1(C)CC[C@]2(C)CC[C@]3(C)C(=CCC4[C@@]5(C)CC[C@H](O)C(C)(C)[C@@H]5CC[C@]43C)[C@@H]2C1 ; COC(=O)[C@@]1(C)CC[C@]2(C)CC[C@]3(C)C(=CCC4[C@@]5(C)CC[C@H](O[Si](C)(C)C(C)(C)C)C(C)(C)[C@@H]5CC[C@]43C)[C@@H]2C1 ; CC1(C)[C@@H](O[Si](C)(C)C(C)(C)C)CC[C@]2(C)C3CC=C4[C@@H]5C[C@@](C)(CO)CC[C@]5(C)CC[C@@]4(C)[C@]3(C)CC[C@@H]12 ; Cc1ccc(S(=O)(=O)OC[C@@]2(C)CC[C@]3(C)CC[C@]4(C)C(=CCC5[C@@]6(C)CC[C@H](O[Si](C)(C)C(C)(C)C)C(C)(C)[C@@H]6CC[C@]54C)[C@@H]3C2)cc1]

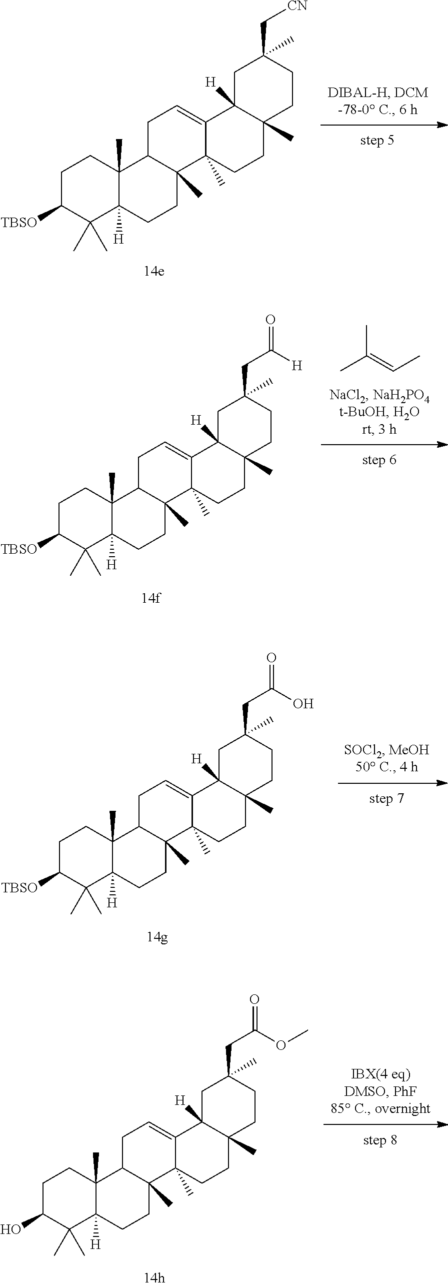

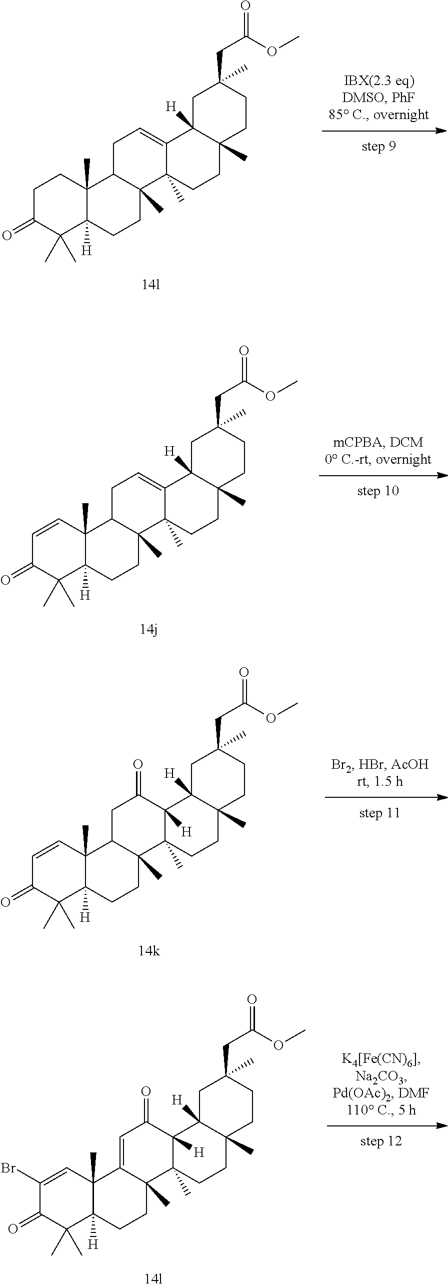

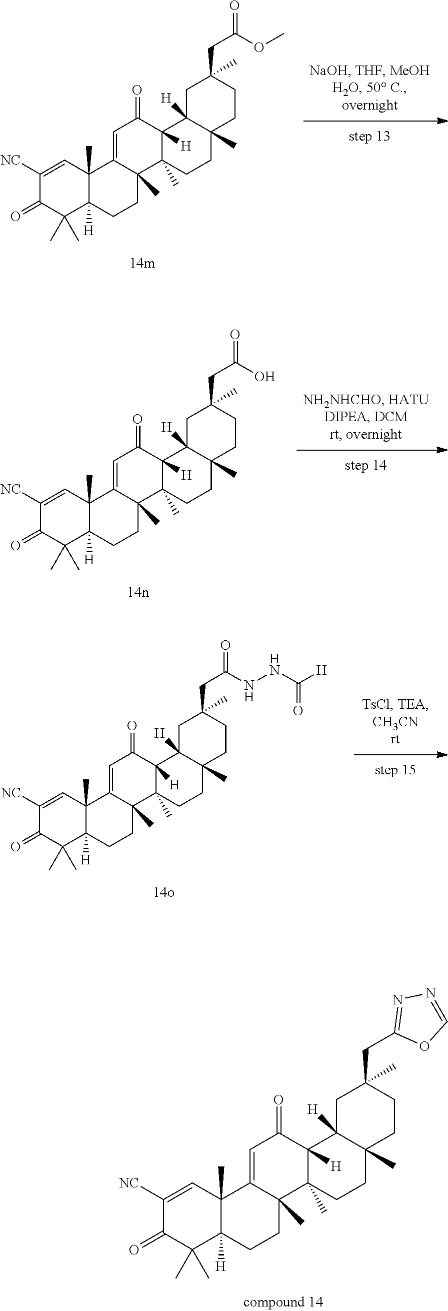

### Step 1: (2S,4aS,6aS,6bR,8aR,10S,12aR,14bR)-methyl-10-((tert-butyldimethylsilypoxy)-2,4a,6a,6b,9,9,12a-heptamethyl-1,2,3,4,4a,5,6,6a,6b,7,8,8a,9,10,11,12,12a,12b,13,14b-icosahydropicene-2-carboxylate (14b)

To a solution of 14a (10.0 g, 21.3 mmol) in DCM (500 mL) was added 2, 6-lutidine (22.8 g, 21.3 mmol) at −60° C. for 0.5 h and added Trifluoromethanesulfonic acid tert-butyldimethylsilyl ester (28.7 g, 106.4 mmol) for another 5 h at this temperature. The mixture was diluted with EA (1000 mL), washed with water (2×500 mL) and brine (1×250 mL), dried with Na₂SO₄ and concentrated. The crude product was purified by flash chromatography to afford the title compound 14b (7.8 g, 63%) as a white solid. LC-MS (ESI): m/z=585.3 [M+H]⁺.

### Step 2: ((2S,4aS,6aS,6bR,8aR,10S,12aR,14bR)-10-((tert-butyldimethylsilypoxy)-2,4a,6a,6b,9,9,12a-heptamethyl- 1,2,3,4,4a,5,6,6a,6b,7,8,8a,9,10,11,12,12a,12b,13,14b-icosahydropicen-2-yl)methanol (14c)

To a solution of 14b (6.3 g, 10.6 mmol) in THF (50 mL) was added Lithium aluminium hydride (2.0 g, 53.0 mmol) at 0° C. for 3 h . The mixture was added water (2 mL) and then added sodium hydroxide (15%, 2 mL) and water (6 mL) at 0° C. The mixture was filtered and the filtrate was diluted with EA (200 mL), washed with water (2×200 mL) and brine (1×250 mL), dried with Na₂SO₄ and concentrated. The crude product was purified by flash chromatography to afford the title compound 14c (4 g, 70.1%) as a yellow oil. LC-MS (ESI): m/z=557.3 [M+H]⁺.

### Step 3: ((2S,4aS,6aS,6bR,8aR,10S,12aR,14bR)-10-((tert-butyldimethylsilypoxy)-2,4a,6a,6b,9,9,12a-heptamethyl- 1,2,3,4,4a,5,6,6a,6b,7,8,8a,9,10,11,12,12a,12b,13,14b-icosahydropicen-2-yl)methyl 4-methylbenzenesulfonate (14d)

To a solution of 14c (4.0 g, 7.0 mmol) in DCM (50 mL) was added tosyl chloride (2.7 g, 14.1 mmol) and triethylamine (3 mL) and DMAP (0.08 g, 0.07 mmol), then the reaction mixture was stirred for 8 h at room temperature. The reaction mixture was then poured into crashed ice, and extracted with ethyl acetate (2×100 mL). The organic layer was dried over magnesium sulfate, filtered, and concentrated in vacuo. The residue was purified by column chromatography on silica gel to give the compound 14d (2.7 g, 52.9%).

LC-MS (ESI): m/z=711.3 [M+H]⁺.

### Step 4: 2-((2S,4aR,6aS,6bR,8aR,10S,12aR,14bR)-10-((tert-butyldimethylsilyl)oxy)-2,4a,6a,6b,9,9,12a-heptamethyl-1,2,3,4,4a,5,6,6a,6b,7,8,8a,9,10,11,12,12a,12b,13,14b-icosahydropicen-2-yl)acetonitrile (14e)

To a solution of 14d (2.7 g, 3.7 mmol) in dimethyl sulfoxide (100 mL) was added potassium cyanide (1.2 g, 18.6 mmol). The reaction mixture was heated at 120° C. for 24 h. After cooling to room temperature, the mixture was diluted with water and EtOAc. The organic layer was separated and the aqueous layer extracted with EtOAc. The combined organic layers were then washed with water and brine, dried over Na₂SO₄, filtered, and concentrated in vacuo. The residue was purified by flash chromatography to afford 14e (1.5 g, 70%) as a yellow solid.

### Step 5: 24(2S,4aR,6aS,6bR,8aR,10S,12aR,14bR)-10-((tert-butyldimethylsilyl)oxy)-2,4a,6a,6b,9,9,12a-heptamethyl-1,2,3,4,4a,5,6,6a,6b,7,8,8a,9,10,11,12,12a,12b,13,14b-icosahydropicen-2-yl)acetaldehyde (14f)

To a solution of 14e (1.8 g, 3.2 mmol) in DCM (20 mL) was added Dibal (2.4 mL, 2 M, 4.8 mmol) at 0° C. The reaction mixture was stirred at 0° C. for 6 h, quenched with saturated solution of sodium potassium tartrate, extracted with DCM (2×20 mL). The combined organic layers were dried over Na₂SO₄, concentrated in vacuo. The residue was purified by flash chromatography to afford 14f (1.1 g, 64%) as a white solid. NMR (400 MHz, CDCl₃) δ 9.81 (t, 1H), 5.15 (t, 1H), 3.17-3.13 (m, 1H), 2.36-2.33 (m, 2H), 2.00-0.72 (m, 53H), 0.00 (t, 6H).

### Step 6: 2-((2S,4aR,6aS,6bR,8aR,10S,12aR,14bR)-10-((tert-butyldimethylsilyl)oxy)-2,4a,6a,6b, 9,9,12a-heptamethyl-1,2,3,4,4a,5,6,6a,6b,7,8,8a,9,10,11,12,12a,12b,13,14b-icosahydropicen-2-yl)acetic acid (14g)

To a solution of 14f (1.1 g, 2.0 mmol) in t-BuOH (10 mL) was added DCM (10 mL), H₂O (2 mL), 2-methylbut-2-ene (0.46 g, 4.6 mmol) and NaH₂PO₄ (0.5 g, 4.5 mmol). The mixture was added NaClO₂ (0.27 g, 4.0 mmol) at 0° C. The reaction mixture was warmed to room temperature and stirred for 3 h, quenched with water (20 mL), and extracted with DCM (2×20 mL). The combined organic layers were dried over Na₂SO₄, concentrated in vacuo. The residue was purified by flash chromatography to afford 14 g (1.0 g, 85%) as a white solid. IH NMR (400 MHz, CDCl₃) δ 5.21 (t, 1H), 3.21-3.17 (m, 1H), 2.41-2.30 (m, 2H), 1.40-0.75 (m, 53H), 0.04 (t, 6H).

### Step 7: methyl 2-((2S,4aR,6aS,6bR,8aR,10S,12aR,14bR)-10-hydroxy-2,4a,6a,6b,9,9,12ahepta methyl-1,2,3,4,4a,5,6,6a,6b,7,8,8a,9,10,11,12,12a,12b,13,14b-icosahydropicen-2-yl)acetate (14h)

To a solution of 14g (2.5 g, 4.3 mmol) in MeOH (30 mL) was added SOCl₂ (1.0 g, 8.6 mmol). The reaction mixture was stirred at 40° C. for 3 h, washed with NaHCO3 (sat.aq, 30 mL) to PH=8-9, and extracted with DCM (2×20 mL). The combined organic layers were dried over Na₂SO₄, concentrated in vacuo. The residue was purified by flash chromatography to afford 14h (1.2 g, 58%) as a white solid. ¹H NMR (400 MHz, CDCl₃) δ 5.21 (t, 1H), 3.65 (s, 3H), 3.24-3.20 (m, 1H), 2.40-2.26 (m, 2H), 1.40-0.75 (m, 44H).

### Step 8: methyl 2-((2S,4aR,6aS,6bR,8aR,12aR,14bR)-2,4a,6a,6b,9,9,12a-heptamethyl-10-oxo-1,2,3,4,4a,5,6,6a,6b,7,8,8a,9,10,11,12,12a,12b,13,14b-icosahydropicen-2-yl)acetate (141)

To a solution of 14h (2.0 g, 4.1 mmol) and PhF (1 mL) in DMSO (20 mL) was added IBX (4.6 g, 17.0 mmol). The reaction mixture was stirred at 85° C. overnight, washed with Na₂S₂O₃ (sat.aq, 30 mL), NaHCO₃ (sat.aq, 30 mL), and extracted with EA (2×20 mL). The combined organic layers were dried over Na₂SO₄, concentrated in vacuo to afford 14i (2.0 g, crude) as a yellow oil used in next step.

### Step 9: methyl 2-((2S,4aR,6aS,6bR,8aR,12aR,14bR)-2,4a,6a,6b,9,9,12a-heptamethyl-10-oxo-1,2,3,4,4a,5,6,6a,6b,7,8,8a,9,10,12a,12b,13,14b-octadecahydropicen-2-yl)acetate (14j)

To a solution of 14i (2.0 g, 4.1 mmol) and PhF (1 mL) in DMSO (20 mL) was added IBX (2.3 g, 8.2 mmol). The reaction mixture was stirred at 85° C. overnight, washed with Na₂S₂O₃ (sat.aq, 30 mL), NaHCO₃ (sat.aq, 30 mL), and extracted with EA (2×20 mL). The combined organic layers were dried over Na₂SO₄, concentrated in vacuo. The residue was purified by flash chromatography to afford 14j (1.2 g, 60%) as a yellow oil. ¹H NMR (400 MHz, CDCl₃) δ 7.03(d, 1H), 5.80(d, 1H), 5.29(t, 1H), 3.66 (s, 3H), 2.38-2.29 (m, 2H), 1.67-0.80 (m, 40H).

### Step 10: methyl 2-((2S,4aR,6aR,6bR,8aR,12aR,14aR,14bS)-2,4a,6a,6b,9,9,12a-heptamethyl- 10,14-dioxo-1,2,3,4,4a,5,6,6a,6b,7,8,8a,9,10,12a,12b,13,14,14a,14b-icosahydropicen-2-yl)acetate (14k)

To a solution of 14j (1.2 g, 2.5 mmol) in DCM (30 mL) was added m-CPBA (0.9 g, 5.0 mmol). The reaction mixture was stirred at 30° C. overnight, washed with Na₂S₂O₃ (sat.aq, 30 mL), and extracted with DCM (2×20 mL). The combined organic layers were dried over Na₂SO₄, concentrated in vacuo. The residue was purified by flash chromatography to afford 14k (0.95 g, 77%) as a white solid. ¹H NMR (400 MHz, CDCl₃) δ 6.93 (d, 1H), 5.83 (d, 1H), 3.73 (s, 3H), 2.85 (d, 1H), 2.45-2.31 (m, 4H), 1.98-0.95 (m, 38H).

### Step 11: methyl 2-((2S,4aR,6aR,6bS,8aR,12aR,14aR,14bS)-11-bromo-2,4a,6a,6b,9,9,12a-heptamethyl-10,14-dioxo-1,2,3,4,4a,5,6,6a,6b,7,8,8a,9,10,12a,14,14a,14b-octadecahydropicen-2-yl)acetate (141)

To a solution of 14k (0.5 g, 1.0 mmol) and HBr (36 mg, 0.4 mmol) in AcOH (5 mL) was added dropwise Br₂ (0.37 g, 2.3 mmol). The reaction mixture was stirred at room temperature for 1.5 h, washed with Na₂S₂O₃ (sat.aq, 20 mL), and extracted with DCM (2×30 mL). The combined organic layers were dried over Na₂SO₄, concentrated in vacuo. The residue was purified by flash chromatography to afford 141 (0.45 g, 78%) as a white solid. LC-MS (ESI): m/z=573.3 [M+H]⁺.

### Step 12: methyl 2-((2S,4aR,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicen-2-yl)acetate (14m)

To a solution of 141 (0.35 g, 0.6 mmol) in DMF (5 mL) was added Na₂CO₃ (0.12 g, 1.2 mmol), potassium ferrocyanide trihyrate (0.15 g, 0.4 mmol) and Pd(OAc)₂ (27 mg, 0.1 mmol). The reaction mixture was stirred under N₂ atmosphere at 110° C. for 5 h, washed with water (20 mL), and extracted with EA (2×30 mL). The combined organic layers were dried over Na₂SO₄, concentrated in vacuo. The residue was purified by flash chromatography to afford 14m (0.10 g, 32%) as a white solid. LC-MS (ESI): m/z=520.3 [M+H]⁺.

### Step 13: 2-((2S,4aR,6aR,6bS,8aR,12aS,14aR,14bS)-11-cyano-2,4a,6a,6b,9,9,12a-heptamethyl- 10,14-dioxo- 1,2,3,4,4a,5,6,6a,6b,7,8,8a,9,10,12a,14,14a,14b-octadecahydropicen-2-yl)acetic acid (14n)

To a solution of 14m (0.27 g, 0.5 mmol) in CH₃OH (3 mL), THF (3 mL) and H₂O (3 mL) was added NaOH (0.21 g, 5.2 mmol). The reaction mixture was stirred at 50° C. overnight, added dropwise HCl (1 N aq.) until PH=5-6, extracted with EA (2×30 mL). The combined organic layers were dried over Na₂SO₄, concentrated in vacuo to afford 14n (0.26 g, 99%) as a white solid. LC-MS (ESI): m/z=506.3 [M+H]⁺.

### Step 14: 2-((2S,4aR,6aR,6bS,8aR,12aS,14aR,14bS)-11-cyano-2,4a,6a,6b,9,9,12a-heptamethyl -10,14-dioxo- 1,2,3,4,4a,5,6,6a,6b,7,8,8a,9,10,12a,14,14a,14b-octadecahydropicen-2-yl)-N′-formylacetohydrazide (14o)

To a solution of 14n (0.26 g, 0.5 mmol) in DCM (5 mL) was added formylhydrazine (42 mg, 0.7 mmol), DIPEA (0.2 g, 1.6 mmol) and HATU (0.26 g, 0.7 mmol). The reaction mixture was stirred at room temperature overnight, washed with water (20 mL), and extracted with DCM (2×20 mL). The combined organic layers were dried over Na₂SO₄, concentrated in vacuo. The residue was purified by flash chromatography to afford 14o (0.16 g, 55%) as a white solid. LC-MS (ESI): m/z=548.4 [M+H]⁺.

### Step 15: (4aR,6aS,6bR,8aR,11S,12aS,12bR,14bS)-11-((1,3,4-oxadiazol-2-yl)methyl)-4,4,6a,6b,8a,11,14b-heptamethyl-3,13-dioxo-3,4,4a,5,6,6a,6b,7,8,8a,9,10,11,12,12a,12b,13,14b-octadecahydropicene-2-carbonitrile (compound 14)

To a solution of 14o (0.16 g, 0.3 mmol) in CH₃CN (5 mL) was added DIPEA (0.11 g, 0.9 mmol) and TsCl (0.17 g, 0.9 mmol). The reaction mixture was stirred at room temperature for 2 h, washed with water (20 mL), and extracted with EA (2×20 mL). The combined organic layers were dried over Na₂SO₄, concentrated in vacuo. The residue was purified by HPLC to afford compound 14 (23 mg, 15%) as a white solid. LC-MS (ESI): m/z=530.3 [M+H]⁺. ¹H NMR (400 MHz, CDCl₃) 67 8.40 (s, 1H), 8.03 (s, 1H), 5.99 (s, 1H), 3.17-3.11 (m, 2H), 2.89-2.86 (m, 1H), 2.28-2.23 (m, 1H), 2.25-0.97 (m, 36H).

### Example 15: (4aR,6aS,6bR,8aS,11S,12aS,12bR,14bS)-4,4,6a,6b,8a,11,14b-heptamethyl-11-(3-methyl-1,2,4-oxadiazol-5-yl)-3,13-dioxo-3,4,4a,5,6,6a,6b,7,8,8a,9,10,11,12,12a,12b,13,14b-octadecahydropicene-2-carbonitrile (compound 15)

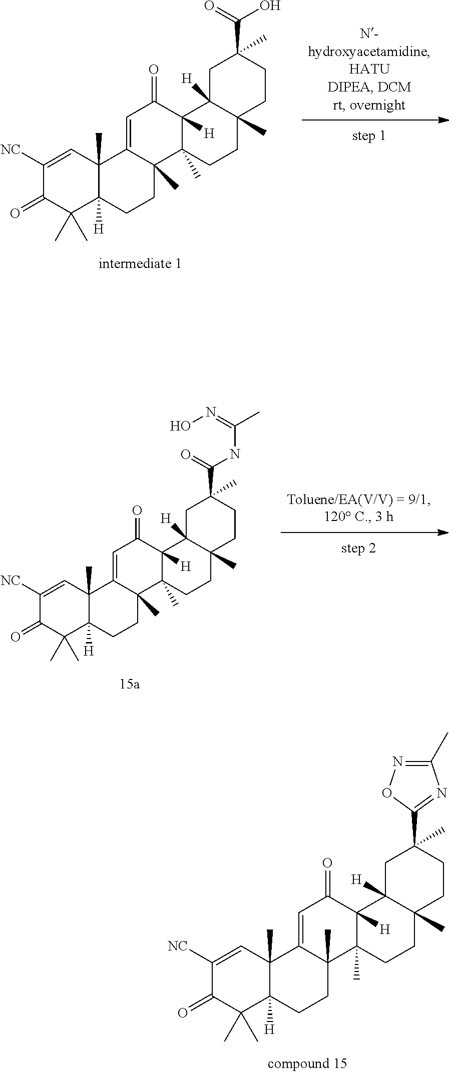

### Step 1: (2S,4aS,6aR,6bS,8aR,12aS,14aR,14bS)-11-cyano-N-((Z)-1-(hydroxyimino)ethyl)-2,4a,6a,6b,9,9,12a-heptamethyl-10,14-dioxo-1,2,3,4,4a,5,6,6a,6b,7,8,8a,9,10,12a,14,14a,14b-octadecahydropicene-2-carboxamide (15a)

To a stirred solution of intermediate 1 (0.50 g, 1.00 mmol), HATU (0.50 g, 1.20 mmol)and N′-hydroxyacetamidine (0.10 g, 2.0 mmol) in DCM (10 mL) at room temperature was added DIPEA (0.4 g, 3.0 mmol) under nitrogen. The resulting solution was stirred at room temperature overnight. Upon consumption of the starting material, the reaction mixture was diluted with EtOAc and washed with brine (20 mL). The organic layer was dried (Na₂SO₄), filtered and concentrated under reduced pressure. The crude product was purified by flash silica chromatography to afford 15a (0.75 g, 100%) as a white solid. LC-MS (ESI): m/z=548.4 [M+H]⁺.

### Step 2: (4aR,6aS,6bR,8aS,11S,12aS,12bR,14bS)-4,4,6a,6b,8a,11,14b-heptamethyl-11-(3-methyl-1,2,4-oxadiazol-5-yl)-3,13-dioxo-3,4,4a,5,6,6a,6b,7,8,8a,9,10,11,12,12a,12b,13,14b-octadecahydropicene-2-carbonitrile (compound 15)

To a solution of 15a (0.70 g, 1.3 mmol) in toluene (13.5 mL) was added EtOAc (1.5 mL). The reaction mixture was stirred at 120° C. for 4 h, and concentrated under reduced pressure. The crude product was purified by flash silica chromatography to afford compound 15 (100 mg, 15%) as a white solid. LC-MS (ESI): m/z=530.3 [M+H]⁺. ¹H NMR (400 MHz, CDCl₃) δ 8.06 (s, 1H), 6.04 (s, 1H), 3.08 (d, 1H), 2.54-2.34 (m, 4H), 2.20 (m, 1H), 2.07 (m, 1H), 2.00-1.67 (m, 6H), 1.64-1.34 (m, 11H), 1.28 (m, 6H), 1.20-1.10 (m, 4H), 1.09-0.95 (m, 4H), 0.91 (s, 3H).

### Example 16: (4aR,6aS,6bR,8aS,11S,12aS,12bR,14bS)-11-(5-cyclopropyl-1,3,4-oxadiazol-2-yl)-4,4,6a,6b,8a,11,14b-heptamethyl-3,13-dioxo-3,4,4a,5,6,6a,6b,7,8,8a,9,10,11,12,12a,12b,13,14b-octadecahydropicene-2-carbonitrile (compound 16)

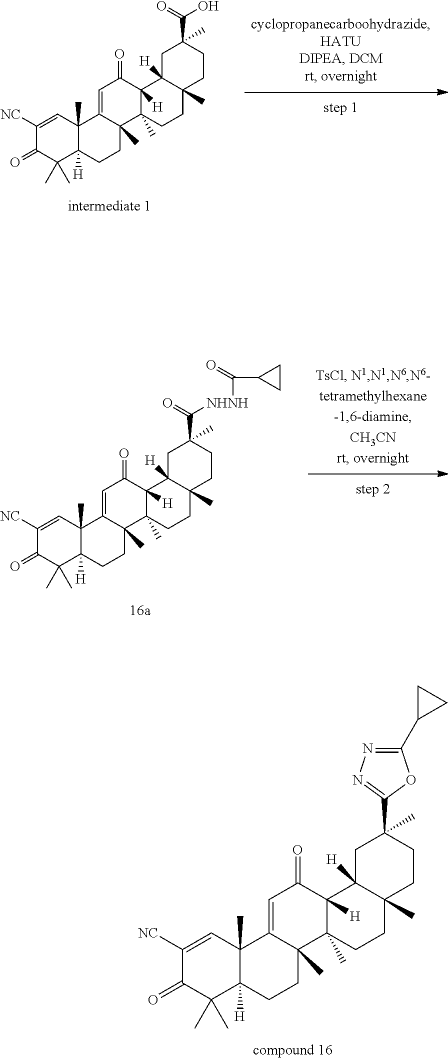

The title compound was prepared by the method substantially similar to that mentioned in compound 9, using cyclopropanecarbohydrazide to afford compound 16 as a white solid. LC-MS (ESI): m/z=556.4 [M+H]⁺. ¹H NMR (400 MHz, CDCl₃) δ 8.03 (s, 1H), 6.03 (s, 1H), 3.03 (d, 1H), 2.28 (m, 3H), 1.99-1.65 (m, 8H), 1.63-1.44 (m, 10H), 1.38 (d, 1H), 1.30-1.09 (m, 13H), 1.00 (d, 4H), 0.90 (s, 3H).

### Example 17: (4aR,6aS,6bR,8aS,11S,12aS,12bR,14bS)-4,4,6a,6b,8a,11,14b-heptamethyl-3,13 dioxo-11-(4H-1,2,4-triazol-4-yl)-3,4,4a,5,6,6a,6b,7,8,8a,9,10,11,12,12a,12b,13,14b-octadecahydropicene-2-carbonitrile (compound 17)

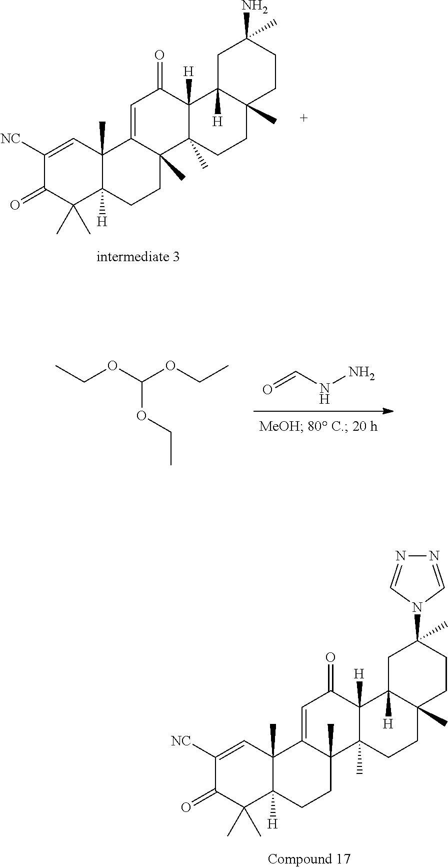

To a solution of Triethoxymethane (0.080 g, 0.54 mmol) and formohydrazide (0.032 g, 0.54 mmol) in MeOH (5 mL) was added intermediate 3 (50 mg, 0.11mmol). The reaction mixture was stirred at 80° C. for 20 h, washed with water (20 mL), and extracted with DCM (2×10 mL). The combined organic layers were dried over Na₂SO₄, concentrated in vacuo. The residue was purified by flash chromatography to afford compound 17 (0.023 g, 41%) as a white solid. LC-MS (ESI): m/z=515.4 [M+H]⁺. ¹H NMR (400 MHz, CDCl₃) δ 9.47 (s, 2H), 8.04 (s, 1H), 6.08 (s, 1H), 3.15 (d, 1H), 2.51 (d, 1H), 2.41 (d, 1H), 2.00 (s, 1H), 2.00 1.71 (m, 9H), 1.62 (d, 1H), 1.54 (d, 9H), 1.27 (d, 4H), 1.20 (s, 4H), 1.13 (d, 1H), 1.03 (s, 3H), 0.90 (d, 3H).

### Example 18: (4aR,6aS,6bR,8aS,11S,12aS,12bR,14bS)-11-(2,5-dioxopyrrolidin-1-yl)-4,4,6a,6b,8a,11,14b-heptamethyl-3,13-dioxo-3,4,4a,5,6,6a,6b,7,8,8a,9,10,11,12,12a,12b,13,14b-octadecahydropicene-2-carbonitrile (compound 18)

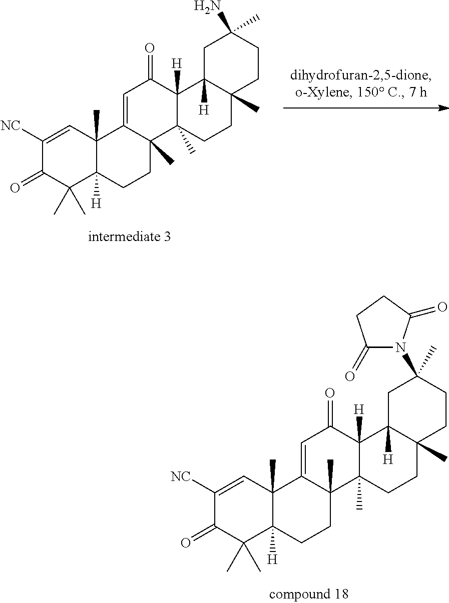

A solution of intermediate 3 (100 mg, 0.21 mmol) in o-xylene (10 mL) was heated to 150° C. The resulting solution was stirred at 150° C. for 7 h. After cooling to room temperature, the reaction mixture was concentrated under reduced pressure. The crude product was purified by flash silica chromatography to afford compound 18 (12 mg, 10%) as a white solid. LC-MS (ESI): m/z =545.3[M+H]⁺. ¹H NMR (400 MHz, CDCl₃) δ 8.03 (s, 1H), 6.06 (s, 1H), 3.09 (t, 3H), 2.77-2.65 (m, 3H), 2.63-2.51 (m, 4H), 1.85 (m, 6H), 1.58 (d, 1H), 1.46 (t, 6H), 1.41-1.30 (m, 5H), 1.25 (d, 4H), 1.21 1.09 (m, 4H), 1.00 (t, 6H).

### Example 19: 5-((2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicen-2-yl)-N-cyclopropyl-1,3,4-oxadiazole-2-carboxamide (compound 19)

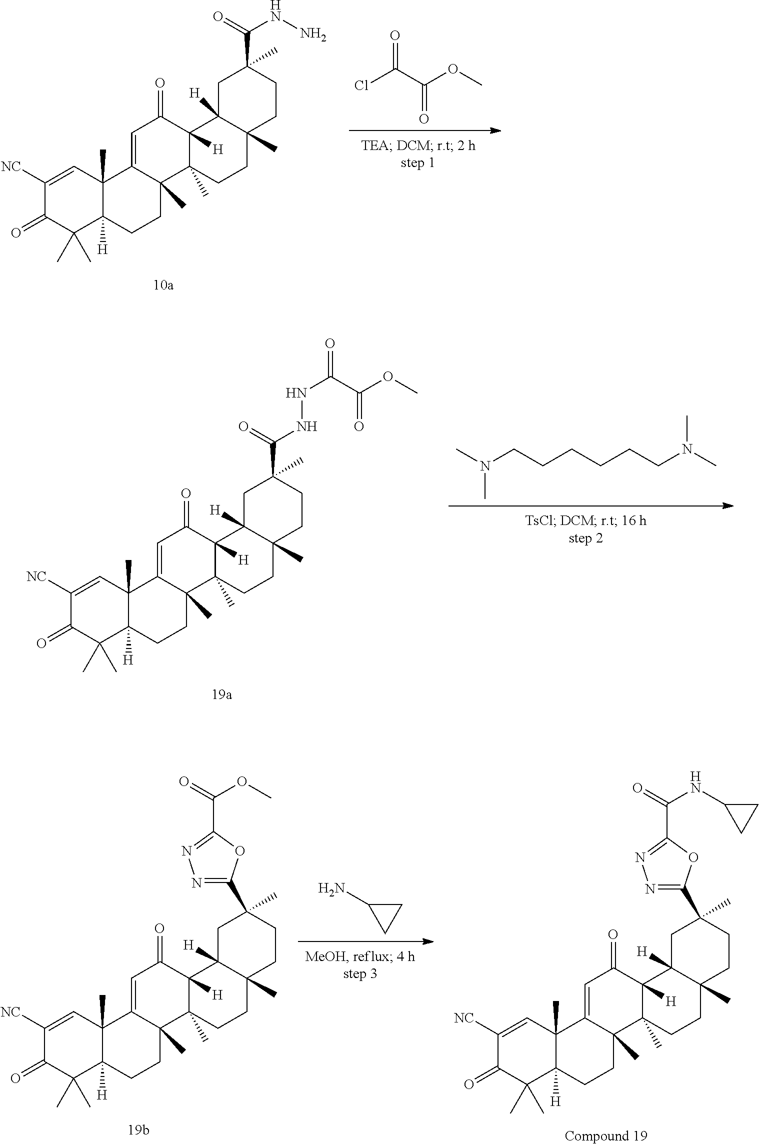

### Step 1: methyl2-(2-((2S,4aS,6aR,6bS,8aR,12aS,14aR,14bS)-11-cyano-2,4a,6a,6b,9,9,12a-heptamethyl- 10,14-dioxo-1,2,3,4,4a,5,6,6a,6b,7,8,8a,9,10,12a,14,14a,14b-octadecahydropicene-2-carbonyl)hydrazineyl)-2-oxoacetate (19a)

To a solution of 10a (0.25 g, 0.49 mmol) and TEA (0.15 g, 1.5 mmol) in DCM (10 mL) was added methyl 2-chloro-2-oxoacetate (0.13 g, 0.99 mmol) at 25° C. The reaction mixture was stirred at 25° C. for 2 h, washed with water (20 mL), and extracted with DCM (2×20 mL). The combined organic layers were concentrated in vacuo. The residue was used to next step directly.

### Step 2: methyl 5-((2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicen-2-yl)-1,3,4-oxadiazole-2-carboxylate (19b)

To a solution of 19a (0.28 g, 0.47 mmol) and N¹,N¹,N⁶,N⁶-tetramethylhexane-1,6-diamine (0.16 g, 0.94 mmol) in DCM (10 mL) was added 4-methylbenzenesulfonyl chloride (0.18 g, 0.94 mmol) at 25° C. The reaction mixture was stirred at 25° C. for 16 h, washed with water (20 mL), and extracted with DCM (2×20 mL). The combined organic layers were concentrated in vacuo. The residue was used to next step directly. LC-MS (ESI): m/z=574.3 [M+H]⁺.

### Step 3: 5-((2S,4aS,6aR,6bS,8aR,12aS,14aR,14bS)-11-cyano-2,4a,6a,6b,9,9,12a-heptamethyl- 10,14-dioxo-1,2,3,4,4a,5,6,6a,6b,7,8,8a,9,10,12a,14,14a,14b-octadecahydropicen-2-yl)-N-cyclopropyl-1,3,4-oxadiazole-2-carboxamide (compound 19)

To a solution of 19b (0.20 g, 0.35 mmol) in MeOH (5 mL) was added cyclopropanamine (0.030 g, 0.52 mmol) at 25° C. The reaction mixture was stirred at 85° C. for 4 h, washed with water (20 mL), and extracted with DCM (2×20 mL). The combined organic layers were dried over Na₂SO₄, concentrated in vacuo. The residue was purified by flash chromatography to afford compound 19 (0.012 g, 5.7%) as a white solid. LC-MS (ESI): m/z=599.4 [M+H]⁺. ¹H NMR (400 MHz, CDCl₃) δ 8.07 (s, 1H), 7.55 (d, 1H), 6.03 (s, 1H), 3.07 2.97 (m, 2H), 2.41 2.23 (m, 2H), 1.99 1.69 (m, 8H), 1.67 1.55 (m, 4H), 1.55-1.45 (m, 6H), 1.45-1.36 (m, 1H), 1.35-1.23 (m, 6H), 1.22-1.10 (m, 4H), 1.02 (d, 4H), 0.95-0.86 (m, 4H), 0.82-0.72 (m, 2H).

### Example 20: (4aR,6aS,6bR,8aS,11S,12aS,12bR,14bS)-11-(3-hydroxypyrrolidin-1-yl)-4,4,6a,6b,8a,11,14b-heptamethyl-3,13-dioxo-3,4,4a,5,6,6a,6b,7,8,8a,9,10,11,12,12a,12b,13,14b-octadecahydropicene-2-carbonitrile (compound 20)

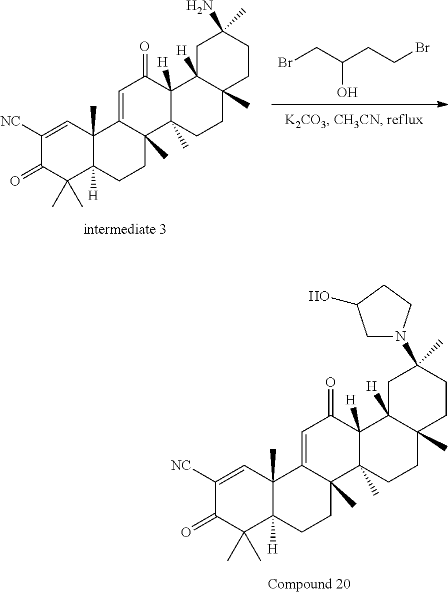 [SMILES: CC1(C)C(=O)C(C#N)=C[C@]2(C)C3=CC(=O)[C@@H]4[C@@H]5C[C@@](C)(N)CC[C@]5(C)CC[C@@]4(C)[C@]3(C)CC[C@@H]12 ; OC(CBr)CCBr ; CC1(C)C(=O)C(C#N)=C[C@]2(C)C3=CC(=O)[C@@H]4[C@@H]5C[C@@](C)(N6CCC(O)C6)CC[C@]5(C)CC[C@@]4(C)[C@]3(C)CC[C@@H]12]

To a solution of intermediate 3 (0.25 g, 0.54 mmol) in CH₃CN (10 mL) was added 1,4-dibromobutan-2-ol (0.25 g, 1.08 mmol), potassium bicarbonateat (0.27 g, 2.70 mmol). The reaction mixture was heated to reflux for 7 h, after cooling to room temperature, the reaction mixture washed with water (20 mL), and extracted with DCM (2×20 mL). The combined organic layers were dried over Na₂SO₄, concentrated in vacuo. The residue was purified by flash chromatography to afford compound 20 (20 mg, 6.9%) as a white solid. LC-MS (ESI): m/z=533.3 [M+H]⁺.

### Example 21: (4aR,6aS,6bR,8aS,11S,12aS,12bR,14bS)-4,4,6a,6b,8a,11,14b-heptamethyl-3,13-dioxo-11-(pyrrolidin-1-yl)-3,4,4a,5,6,6a,6b,7,8,8a,9,10,11,12,12a,12b,13,14b-octadecahydropicene-2-carbonitrile (compound 21)

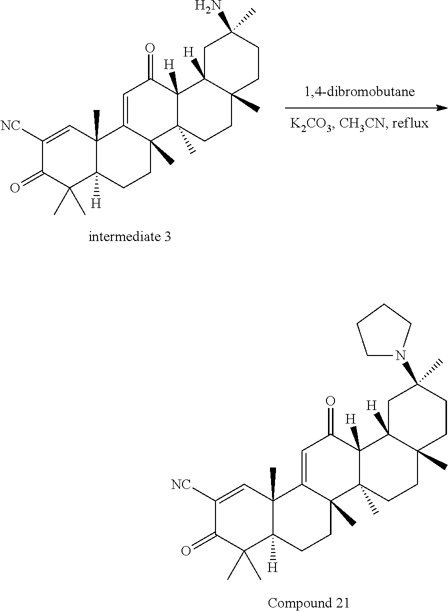 [SMILES: CC1(C)C(=O)C(C#N)=C[C@]2(C)C3=CC(=O)[C@@H]4[C@@H]5C[C@@](C)(N)CC[C@]5(C)CC[C@@]4(C)[C@]3(C)CC[C@@H]12 ; CC1(C)C(=O)C(C#N)=C[C@]2(C)C3=CC(=O)[C@@H]4[C@@H]5C[C@@](C)(N6CCCC6)CC[C@]5(C)CC[C@@]4(C)[C@]3(C)CC[C@@H]12]

The title compound was prepared by the method substantially similar to that mentioned in compound 20, using 1,4-dibromobutane to afford compound 21 (19 mg, 5.7%) as a white solid. LC-MS (ESI): m/z=517.4 [M+H]⁺.

### Example 23: (4aR,6aS,6bR,8aS,11S,12aS,12bR,14bS)-11-(5-((S)-1-hydroxyethyl)-1,3,4-oxadiazol-2-yl)-4,4,6a,6b,8a,11,14b-heptamethyl-3,13-dioxo-3,4,4a,5,6,6a,6b,7,8,8a,9,10,11,12,12a,12b,13,14b-octadecahydropicene-2-carbonitrile (compound 23)

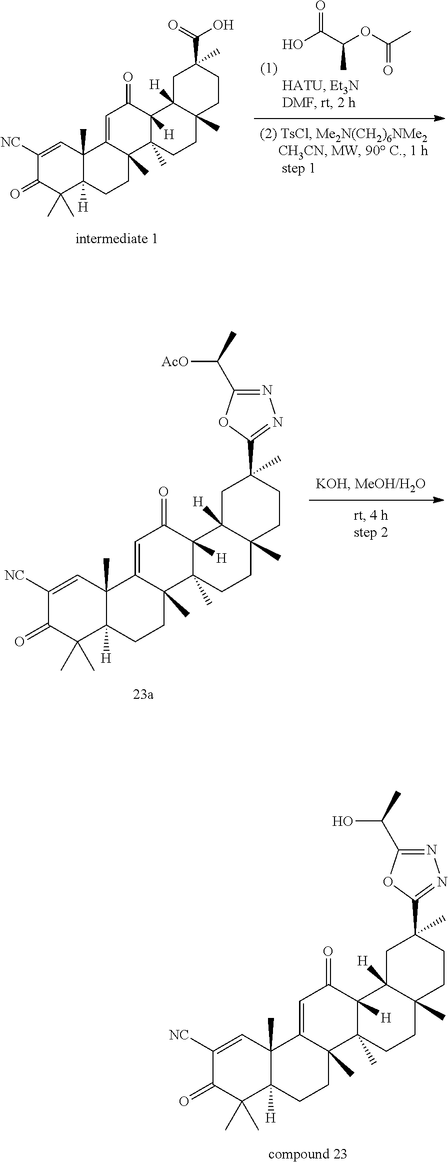

### Step 1: (S)-1-(5-((2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicen-2-yl)-1,3,4-oxadiazol-2-ypethyl acetate (23a)

23a was prepared by the method substantially similar to that mentioned in compound 1, using (S)-2-acetoxypropanoic acid to afford compound 23a (62 mg, 62%) as a white solid. LC-MS (ESI): m/z=602.4 [M+H]⁺.

### Step 2: (4aR,6aS,6bR,8aS,11S,12aS,12bR,14bS)-11-(54(S)-1-hydroxyethyl)-1,3,4-oxadiazol -2-yl)-4,4,6a,6b,8a,11,14b-heptamethyl-3,13-dioxo-3,4,4a,5,6,6a,6b,7,8,8a,9,10,11,12,12a,12b,13,14b-octadecahydropicene-2-carbonitrile (compound 23)

To a solution of 23a (62 mg, 0.10 mmol) in MeOH (5mL), H₂O (1 mL) was added Potassium hydroxide (11m g, 0.19 mmol). The reaction mixture was stirred at room temperature for 4 h. the reaction mixture washed with water (20 mL), and extracted with DCM (2×20 mL). The combined organic layers were dried over Na₂SO₄, concentrated in vacuo. The residue was purified by flash chromatography to afford compound 23 (18 mg, 32.3%) as a white solid. LC-MS (ESI): m/z=560.3 [M+H]⁺.

### Example 24 : 5-((2S,4aS,6aR,6bS,8aR,12aS,14aR,14bS)-11-cyano-2,4a,6a,6b,9,9,12a- heptamethyl-10,14-dioxo-1,2,3,4,4a,5,6,6a,6b,7,8,8a,9,10,12a,14,14a,14b-octadecahydropicen-2-yl)-N-methyl-1,3,4-oxadiazole-2-carboxamide (compound 24)

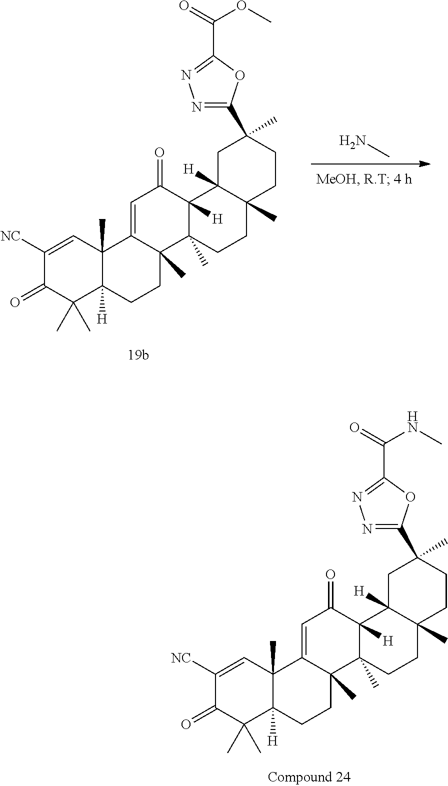

To a solution of 19b (0.05 g, 0.087 mmol) in MeOH (2 mL) was added methanamine (0.011 g, 0.35 mmol) at 25° C. The reaction mixture was stirred at 25° C. for 4 h, washed with water (10 mL), and extracted with DCM (2×10 mL). The combined organic layers were dried over Na₂SO₄, concentrated in vacuo. The residue was purified by flash chromatography to afford compound 24 (3.0 mg, 6.0%) as a white solid. LC-MS (ESI): m/z=573.3 [M+H]⁺. ¹ H NMR (400 MHz, CDCl₃) δ 8.07 (s, 1H), 7.52 (d, 1H), 6.07 (s, 1H), 3.08 (t, 3H), 2.38 (d, 1H), 2.34 2.26 (m, 1H), 1.97 1.72 (m, 8H), 1.64-1.60 (m, 4H), 1.56-1.50 (m, 4H), 1.48 (s, 3H), 1.45 1.39 (m, 1H), 1.33 (s, 3H), 1.28 (s, 3H), 1.19 (s, 3H), 1.16 (d, 1H), 1.06-1.01 (m, 3H), 0.90 (d, 3H).

### Example 25: 5-((2S,4aS,6aR,6bS,8aR,12aS,14aR,14bS)-11-cyano-2,4a,6a,6b,9,9,12a- heptamethyl-10,14-dioxo-1,2,3,4,4a,5,6,6a,6b,7,8,8a,9,10,12a,14,14a,14b-octadecahydropicen-2-yl)-1,3,4-oxadiazole-2-carboxamide (compound 25)

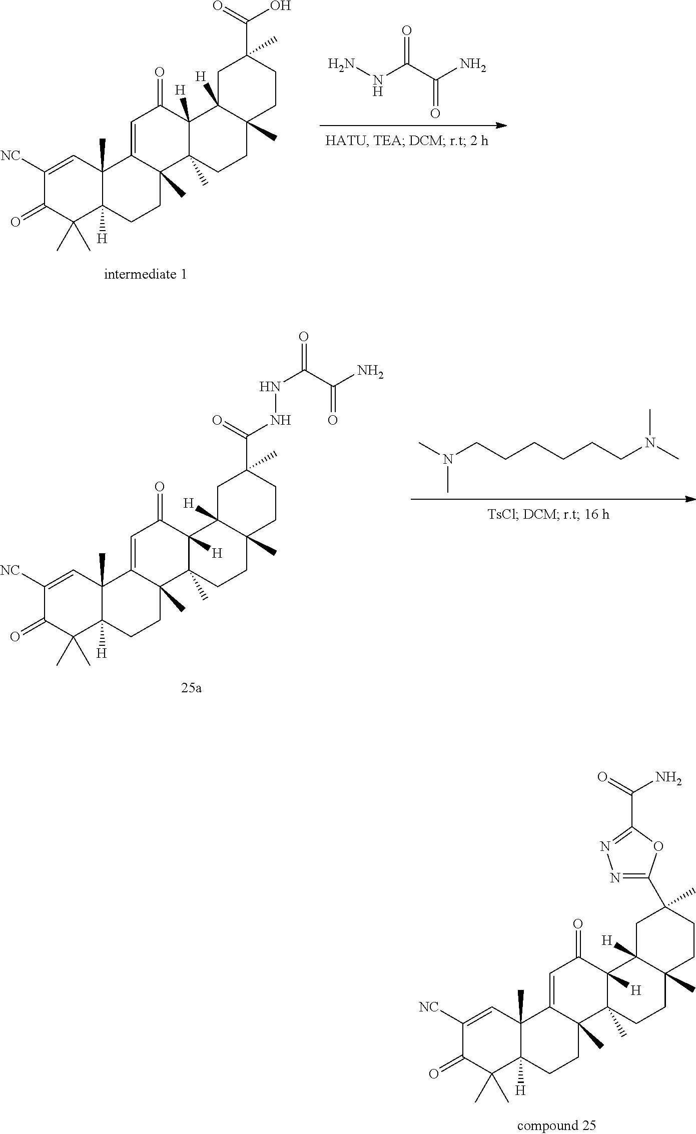

### Step 1: 2-(2-((2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicene-2-carbonyl)hydrazinyl)-2-oxoacetamide (25a)

To a solution of intermediate 1 (0.100 g, 0.203 mmol), HATU(0.093 g, 0.244 mmol) and DIPEA (0.080 g, 0.61 mmol) in DCM (10 mL) was added 2-hydrazinyl-2-oxoacetamide (0.031g, 0.305 mmol) at 25° C. The reaction mixture was stirred at 25° C. for 2 h, washed with water (20 mL), and extracted with DCM (2×20 mL). The combined organic layers were concentrated in vacuo. The residue was used to next step directly.

### Step 2: 5-((2S,4aS,6aR,6bS,8aR,12aS,14aR,14bS)-11-cyano-2,4a,6a,6b,9,9,12a-heptamethyl-10,14-dioxo-1,2,3,4,4a,5,6,6a,6b,7,8,8a,9,10,12a,14,14a,14b-octadecahydropicen-2-yl)-1,3,4-oxadiazole-2-carboxamide (25)

To a solution of 25a (0.10 g, 0.17 mmol) and N¹,N¹,N⁶,N⁶-tetramethylhexane-1,6-diamine (0.059 g, 0.35 mmol) in DCM (5 mL) was added 4-methylbenzenesulfonyl chloride (0.66 g, 0.35 mmol) at 25° C. The reaction mixture was stirred at 25° C. for 16 h, washed with water (20 mL), extracted with DCM (2×20 mL). The combined organic layers were concentrated in vacuo. The residue was purified by flash chromatography to afford compound 25 (0.030 g, 31%) as a white solid. LC-MS (ESI): m/z=559.4 [M+H]⁺. ¹H NMR (400 MHz, CDCl₃) δ 8.07 (s, 1H), 7.54 (s, 1H), 6.40 (s, 1H), 6.09 (s, 1H), 3.07 (d, 1H), 2.38-2.32 (m, 2H), 1.92-1.88 (m, 3H), 1.85 1.73 (m, 4H), 1.64-1.60 (m, 4H), 1.53 (d, J=6.2 Hz, 3H), 1.48 (s, 3H), 1.44-1.38 (m, 1H), 1.34 (s, 3H), 1.27 (s, 3H), 1.20-1.13 (m, 4H), 1.04 (d, 4H), 0.91 (s, 3H).

### Example 26: (4aR,6aS,6bR,8aS,11S,12aS,14bS)-11-(3-hydroxyazetidin-1-yl)-4,4, 6a,6b,8a,11,14b- heptamethyl-3,13-dioxo-3,4,4a,5,6,6a,6b,7,8,8a,9,10,11,12,12a,12b,13,14b-octadecahydropicene-2-carbonitrile (compound 26)

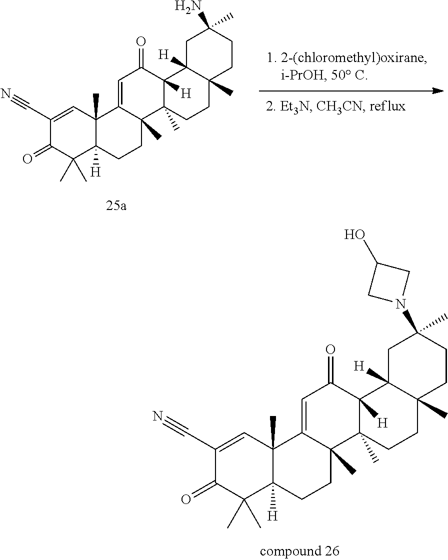

To a solution of intermediate 3 (50 mg, 0.11 mmol) in i-PrOH (2 mL) was added 2-(chloro methyl)oxirane (11 mg, 0.11 mmol). The reaction mixture was stirred at 50° C. overnight and concentrated in vacuo. The residue was added CH₃CN (5 mL), Et₃N (27 mg, 0.27 mmol). The reaction mixture was refluxed for 4 h. The reaction mixture was washed with water (20 mL), extracted with DCM (2×20 mL). The combined organic layers were dried over Na₂SO₄, concentrated in vacuo. The residue was purified by flash chromatography to afford compound 26 (16 mg, 34.3%) as a white solid. LC-MS (ESI): m/z=519.4 [M+H]⁺. ¹H NMR (400 MHz, CDCl₃) δ 8.06 (s, 1H), 6.03 (s, 1H), 4.64-3.80 (m, 5H), 3.14 (s, 1H), 2.19 (d, 2H), 2.09 0.75 (m, 35H).

### Example 27: (4aR,6aS,6bR,8aS,11S,12aS,12bR,14bS)-11-(5-amino-1,3,4-oxadiazol-2-yl)-4,4,6a,6b,8a,11,14b-heptamethyl-3,13-dioxo-3,4,4a,5,6,6a,6b,7,8,8a,9,10,11,12,12a,12b,13,14b-octadecahydropicene-2-carbonitrile (compound 27)

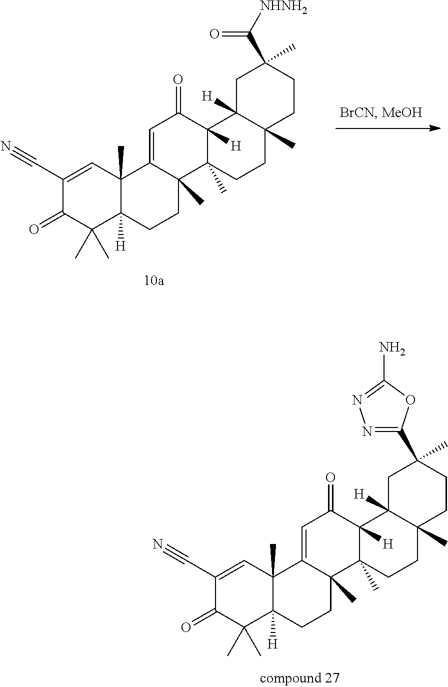

To a solution of 10a (50 mg, 0.10 mmol) in MeOH (2 mL) was added cyanogen bromide (21 mg, 0.20 mmol). The reaction mixture was stirred at room temperature overnight and washed with water (20 mL), extracted with DCM (2×20 mL). The combined organic layers were dried over Na₂SO₄, concentrated in vacuo. The residue was purified by flash chromatography to afford compound 27 (2.7 mg, 5.1%) as a white solid. LC-MS (ESI): m/z=531.3 [M+H]⁺. ¹H NMR (400 MHz, CDCl₃) 67 8.02 (s, 1H), 5.99 (s, 1H), 3.07 (m, 1H), 1.82 (m, 8H), 1.67-1.37 (m, 10H), 1.39-1.23 (m, 9H), 1.21-1.09 (m, 4H), 1.01 (m, 6H).

### Example A: Nitric Oxide Assays

### Nitric Oxide Assay

Raw 264.7 cells were seeded in 96-well plates at a density of 30, 000 cells per well in DMEM+10% FBS and incubated overnight. The next day, cell media was replaced by DMEM+2% FBS. Cells were pretreated with DMSO or test compounds (top dose 100 nM, 1:4 serial dilution, 10-point) for 2 hours and then treated with recombinant mouse IFNγ (R&D, Cat# 485-MI-100) at a final concentration of 10 ng/ml for 24 hours. Nitrite levels were measured in culture media as a surrogate for nitric oxide using the Griess Reagent System (Promega, Cat# G2930). Cell viability was determined using CellTiter Glo reagents (Promega, Cat#G7572). The nitrite levels were adjusted to live cell numbers and inhibition percentage was calculated. The inhibition percentage was then plotted against compound concentration, and IC₅₀ values were calculated using four-parameter algorithm in Graphpad Prism.

### NQO1 Activity Assay

Raw264.7 cells were seeded in DMEM+10% FBS in 96-well plates at a density of 10,000 cells per well. 6 hours after plating, cells were treated with DMSO or test compounds (top dose 100 nM, 1:4 serial dilution, 8-point) for 48 hours. NQO1 activity was then detected in cell lysates using NQO1 activity assay kit (Abeam, Cat#ab184867). NQO1 activity was then plotted against compound concentration, and EC₅₀ values were calculated using four-parameter algorithm in Graphpad Prism.

| | Suppression of INFγ- | | |
|---|---|---|---|
| | induced NO production | | |
| | NO IC₅₀ | Relative NO | NQO1 EC₅₀ |
| Ex. | (nM) | IC₅₀ | (nM) |
| Control | A | 1.0 | A |
| 1 | A | 0.7 | A |
| 2 | | | A |
| 3 | A | 1.0 | B |
| 4 | A | 0.8 | B |
| 5 | A | 1.6 | B |
| 6 | A | 0.9 | |
| 7 | A | 0.9 | |
| 8 | B | 32.7 | |
| 9 | A | 0.8 | |
| 10 | A | 1.3 | |
| 11 | A | 1.1 | |
| 12 | A | 0.8 | |
| 13 | A | 0.8 | |
| 14 | A | 0.9 | |
| 15 | A | 0.8 | |
| 16 | A | 0.8 | |
| 18 | A | 0.9 | |
| 19 | A | 1.0 | |
| 20 | A | 0.8 | A |
| 21 | B | 1.3 | |
| 23 | B | 1.8 | |
| 24 | A | 0.5 | |
| 25 | A | 1.0 | B |
| 26 | A | 0.6 | A |
| 27 | B | 5.1 | |

### A<10 nM

10 nM≤B<100 nM
100 nM≤C<1

### Example B: CYP450 Enzyme Inhibition

The inhibition potential of test compounds towards major drug metabolizing CYP450 enzymes was studied in liver microsomes of CD-1 mouse, Sprague Dawley rat, Beagle dog, cynomolgus monkey, and human. The inhibition of CYP1A2, CYP2C9, CYP2C19, CYP2D6, and CYP3A4 was assessed using CYP450 specific probe reactions in a cocktail incubation. Seven concentrations of test compounds (0.05, 0.15, 0.5, 1.5, 5, 15, and 50 μM) were evaluated. In addition, known inhibitors for each probe reaction were included as positive controls and incubated at a single concentration above their respective IC₅₀. The IC50 data (in nM) for each test compound in each species were summarized in the following table.

| Example | CYP1A2 | CYP2C9 | CYP2C19 | CYP2D6 | CYP3A4-M |
|---|---|---|---|---|---|
| Control | >50 | 1.7 | 5.2 | >50 | 2.6 |
| 1 | >50 | 2.7 | 11.8 | 15.0 | 1.0 |
| 2 | >50 | 2.2 | 25.4 | 20.2 | 1.7 |
| 3 | >50 | 3.4 | 17.8 | 4.7 | 6.7 |
| 4 | >50 | 1.6 | 4.5 | 2.2 | 3.3 |
| 6 | >50 | 2.1 | 5.6 | 6.9 | 0.5 |
| 7 | >50 | 2.0 | 9.4 | 10.1 | 0.8 |
| 9 | >50 | 3.1 | 13.3 | 28.7 | 0.3 |
| 10 | >50 | 1.8 | 27.5 | >50 | 1.3 |
| 11 | >50 | 2.2 | 14.6 | >50 | 0.5 |
| 12 | >50 | 2.7 | 9.7 | >50 | 2.8 |
| 13 | >50 | 1.9 | 11.7 | >50 | 1.9 |
| 14 | >50 | 3.0 | 6.2 | 10.3 | 1.0 |
| 15 | >50 | 2.6 | 13.6 | 36.2 | 1.6 |
| 16 | >50 | 2.0 | 7.4 | 14.7 | 1.5 |
| 17 | >50 | 2.2 | 16.9 | 35.4 | 0.1 |
| 18 | >50 | 4.7 | 19.6 | >50 | 2.1 |
| 19 | >50 | 1.2 | 5.8 | 8.4 | 2.1 |
| 20 | >50 | 8.6 | 18.9 | >50 | 3.2 |
| 23 | >50 | 1.5 | 11.8 | 15.8 | 0.9 |
| 24 | >50 | 0.6 | 2.8 | 4.0 | 0.5 |
| 25 | >50 | 6.9 | >50 | >50 | 6.6 |
| 26 | >50 | 27.6 | >50 | >50 | 14.6 |

### Example C: Metabolic Stability of Test Compounds in Mouse, Rat, Dog, Monkey, and Human Liver Microsomes

The metabolic stability of test compounds was examined in CD-1 mouse, Sprague Dawley rat, Beagle dog, cynomolgus monkey, and human liver microsomes. Test compound (final concentration 1 μM) was mixed with diluted liver microsomes from each of the 5 species and a small aliquot was taken at 0, 5, 10, 20, 30, and 60 minutes for LC/MS/MS analysis. Intrinsic clearance and half-life were calculated.

| | Human | | Rat | | Mouse | |
|---|---|---|---|---|---|---|
| Compound | T_{1/2} | CLᵢₙₜ₍ₗᵢᵥₑᵣ₎ | T_{1/2} | CLᵢₙₜ₍ₗᵢᵥₑᵣ₎ | T_{1/2} | CLᵢₙₜ₍ₗᵢᵥₑᵣ₎ |
| ID | (min) | (mL/min/kg) | (min) | (mL/min/kg) | (min) | (mL/min/kg) |
| Control | 13.1 | 95.4 | 3.0 | 831.9 | 30.6 | 179.6 |
| 1 | 7.4 | 167.7 | 59.4 | 42.0 | 13.0 | 423.2 |
| 2 | 3.1 | 397.1 | 15.9 | 157.4 | 3.1 | 1753.2 |
| 3 | 24.6 | 50.7 | 49.6 | 50.3 | 36.5 | 150.5 |
| 4 | 36.5 | 34.2 | 64.3 | 38.8 | 37.3 | 147.3 |
| 9 | 13.3 | 94.0 | 34.6 | 72.1 | 29.5 | 186.3 |
| 14 | 14.2 | 87.9 | 3.6 | 694.9 | 3.8 | 1460.7 |
| 25 | 13.0 | 96.2 | 28.6 | 87.2 | 19.0 | 289.1 |
| 26 | 6.8 | 183.8 | 15.5 | 161.2 | 13.7 | 402.1 |

| | |
|---|---|
| Dog | Monkey |

| Compound | T_{1/2} | CLᵢₙₜ₍ₗᵢᵥₑᵣ₎ | T_{1/2} | CLᵢₙₜ₍ₗᵢᵥₑᵣ₎ |
|---|---|---|---|---|
| ID | (min) | (mL/min/kg) | (min) | (mL/min/kg) |
| Control | 25.6 | 78.0 | 2.0 | 949.4 |
| 1 | 54.0 | 37.0 | 7.0 | 269.2 |
| 2 | 6.4 | 312.8 | 2.4 | 782.2 |
| 3 | 24.5 | 81.6 | 12.0 | 155.8 |
| 4 | 19.7 | 101.1 | 18.1 | 103.5 |
| 9 | 19.1 | 104.5 | 6.9 | 270.0 |
| 14 | 8.8 | 228.0 | 3.1 | 602.2 |
| 25 | 34.2 | 58.4 | 6.4 | 293.7 |
| 26 | 25.8 | 77.3 | 2.5 | 752.4 |

### Example D: Pharmacokinetic Studies in Sprague Dawley Rats

The pharmacokinetics of test compounds was evaluated in male SD rats when administered via oral gavage and IV injection. For oral dosing, compound was formulated in 0.5% methylcellulose and administered at doses of 5 mg/kg (3 rats per dose level) and volume of 10 mL/kg. For IV dosing, compound was formulated in 5% DMSO/5% Solutol/90% saline and administered to 3 rats at a dose of 1 mg/kg and a volume of 5 mL/kg. The rats were fasted overnight before administration. Plasma samples were collected predose and at 0.5, 1, 3, 6, 9, 12, and 24 hours postdose. The samples were analyzed by LC/MS/MS and the concentration of test compound at each timepoint was determined by linear regression. Pharmacokinetic parameters were calculated from the plasma concentrations using Pheonix WinNonlin. The PK results were summarized in the following table.

| | Oral (5 mg/kg) | | | IV (1 mg/kg) | | | | |
|---|---|---|---|---|---|---|---|---|
| | Cₘₐₓ | AUC_{0-24 h} | T_{1/2} | C₀ | AUC_{0-24 h} | T_{1/2} | Clearance | Bioavailibility |
| Ex. | (ng/mL) | (h · ng · mL⁻¹) | (h) | (ng/mL) | (h · ng · mL⁻¹) | (h) | (mL · kg−1 · min−1) | F % |
| Control | 7 | 26 | 3.4 | 365 | 291 | 6.3 | 56.8 | 1.8 |
| 1 | 170 | 1097 | 2.2 | 349 | 258 | 1.7 | 63.8 | 83.8 |
| 2 | 177 | 521 | 1.3 | 500 | 314 | 1.2 | 53.0 | 33.2 |
| 4 | 51 | 214 | 2.9 | 747 | 292 | 3.2 | 50.9 | 14.6 |
| 9 | 143 | 333 | 2.2 | 427 | 301 | 1.5 | 56.0 | 22.1 |
| 12 | 29 | 94 | 4.1 | 359 | 191 | 1.7 | 85.7 | 9.9 |
| 13 | 45 | 197 | 2.1 | 355 | 206 | 2.2 | 78.9 | 19.2 |

| | Oral (5 mg/kg) | | | IV (1 mg/kg) | | | | |
|---|---|---|---|---|---|---|---|---|
| | Cₘₐₓ | AUC_{0-24 h} | T_{1/2} | C₀ | AUC_{0-24 h} | T_{1/2} | Clearance | Bioavailibility |
| Ex. | (ng/mL) | (h · ng · mL⁻¹) | (h) | (ng/mL) | (h · ng · mL⁻¹) | (h) | (mL · kg−1 · min−1) | F % |
| 15 | 89 | 412 | 1.7 | 380 | 329 | 2.8 | 47.0 | 25.0 |
| 18 | 50 | 181 | 2.3 | 472 | 267 | 1.4 | 62.5 | 13.6 |
| 19 | 63 | 364 | 4.5 | 703 | 496 | 1.8 | 32.5 | 14.7 |
| 25 | 32 | 102 | 1.3 | 1302 | 263 | 1.4 | 63.3 | 7.8 |
| 26 | 57 | 270 | 3.6 | 1040 | 294 | 4.7 | 56.3 | 18.3 |

### Example E: Pharmaceutical Compositions

### Example E1: Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example E2: Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example E3: Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

The examples and embodiments described herein are for illustrative purposes only and in some embodiments, various modifications or changes are to be included within the purview of disclosure and scope of the appended claims.

What is claimed is:

1. A compound of Formula (III), or a pharmaceutically acceptable salt, solvate, or stereoisomer thereof: 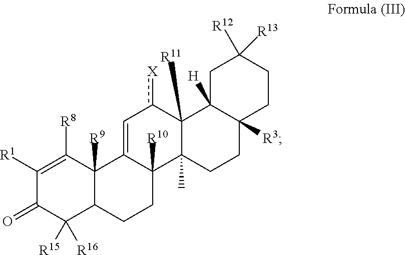
wherein:
R¹ is hydrogen, deuterium, halogen, —CN, —OR^{b}, —NR^{c}R^{d}, —NR^{b}S (═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R³ is N-linked heterocycloalkyl, N-linked heteroaryl, —P(═O)(R⁴)₂, —P(═O)(OR⁵)₂, —B(OR⁵)₂, —S(═O)R⁴, —S(═O)₂R⁴, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S (═O) (═NR^{x}) R⁵, —Z—O-cycloalkyl, —Z—O- heterocycloalkyl, —Z—O-aryl, —Z—O-heteroaryl, —Z—NR⁵-cycloalkyl, —Z—NR⁵-heterocycloalkyl, —Z—NR⁵-aryl, —Z—NR⁵-heteroaryl, —Y(C₁-C₆alkylene)S(═O)R⁴, —Y(C₁-C₆alkylene)S(═O)R⁴, —Y(C₁-C₆alkylene)P(═O)(R⁴)₂, —Y(C₁-C₆alkylene)P(═O)(OR⁵)₂, —Y(C₁-C₆alkylene)B(OR⁵)₂, —Y(C₁-C₆alkylene)NR⁵C(═NR^{x})R⁵, —Y(C₁-C₆alkylene)NR⁵C(═NR^{x})NR⁶R⁷, —Y(C₁-C₆alkylene)S(═O)(═NR^{x})R⁵, —Y(C₁-C₆alkylene)S(═O)(═NR^{x})NR⁶R⁷, —Y(C₁-C₆alkylene)NR⁵S(═O)₂NR⁵C(═O)R⁵, —NR⁵C(═O)(C₁-C₆alkylene)S(═NR^{x})NR⁶R⁷, —NR⁵C(═O)(C₁-C₆alkylene)S(═NR^{x})R⁵, —Y(C₁-C₆alkylene)NR⁵S(═O)(═NR^{x})R⁵, —Y(C₂-C₆alkenylene)S(═O)₂R⁴, —Y(C₂-C₆alkenylene)P(═O)(R⁴)₂, —Y(C₂-C₆alkenylene)P(═O)(OR⁵)₂, —Y(C₂-C₆alkenylene)B(OR⁵)₂, —Y(C₂-C₆alkenylene)NR⁵C(═NR^{x})R⁵, —Y(C₂-C₆alkenylene)NR⁵C(═NR^{x})NR⁶R⁷, —Y(C₂-C₆alkenylene)S(═O)(═NR^{x})R⁵, —Y(C₂-C₆alkenylene)S(═O)(═NR^{x})NR⁶R⁷, —Y(C₂-C₆alkenylene)NR⁵S(═O)₂NR⁵C(═O)R⁵, —Y(C₂-C₆alkenylene)NR⁵S(═O)(═NR^{x})R⁵, —Y(C₂-C₆alkenylene)cycloalkyl, —Y(C₂-C₆alkenylene)heterocycloalkyl, —Y(C₂-C₆alkenylene)aryl, —Y(C₂-C₆alkenylene)heteroaryl, —(C₁-C₆alkylene)OP(═O)(OR⁵)₂, —(C₁-C₆alkylene)O(C₁-C₆alkylene)OP(═O)(OR⁵)₂, or —(C₁-C₆alkylene)OP(═O)(OR⁵)[N(R⁵)₂]; wherein the alkylene, alkenylene, aryl, heteroaryl, cycloalkyl, and heterocycloalkyl are independently optionally substituted with one, two, or three R^{3a};
each R^{3a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
Y is a bond, —O—, —S—, or —NR^{b}—;
Z is a bond or C₁-C₆alkylene;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{4a};
each R^{4a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; 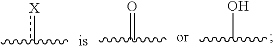
R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R¹¹ are taken together to form an optionally substituted cycloalkyl or an optionally substituted heterocycloalkyl;
R¹² and R¹³ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹² and R¹³ are taken together to form a cycloalkyl or a heterocycloalkyl; each substituted with 0-6 R¹⁴;
each R¹⁴ is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
R¹⁵ and R¹⁶ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹⁵ and R¹⁶ are taken together to form a cycloalkyl or a heterocycloalkyl; each substituted with 0-6 R²;
each R² is independently deuterium, halogen, —CN, —OR^{b}, −NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

2. The compound of claim 1, or a pharmaceutically acceptable salt, solvate, or stereoisomer thereof, wherein:
R¹ is —CN, C₁-C₆ alkyl, C₁-C₆ haloalkyl, or C₁-C₆ deuteroalkyl.

3. The compound of claim 1 or 2, or a pharmaceutically acceptable salt, solvate, or stereoisomer thereof, wherein:
R¹ is —CN.

4. The compound of any one of claims 1-3, or a pharmaceutically acceptable salt, solvate, or stereoisomer thereof, wherein:
R⁸ is hydrogen or deuterium.

5. The compound of any one of claims 1-4, or a pharmaceutically acceptable salt, solvate, or stereoisomer thereof, wherein:
R⁸ is hydrogen.

6. The compound of any one of claims 1-5, or a pharmaceutically acceptable salt, solvate, or stereoisomer thereof, wherein:
R⁹ is C₁-C₆ alkyl.

7. The compound of any one of claims 1-6, or a pharmaceutically acceptable salt, solvate, or stereoisomer thereof, wherein:
R¹⁹ is C₁-C₆ alkyl.

8. The compound of any one of claims 1-7, or a pharmaceutically acceptable salt, solvate, or stereoisomer thereof, wherein:

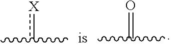

9. The compound of any one of claims 1-8, or a pharmaceutically acceptable salt, solvate, or stereoisomer thereof, wherein:
R¹¹ is hydrogen.

10. The compound of any one of claims 1-9, or a pharmaceutically acceptable salt, solvate, or stereoisomer thereof, wherein:
R¹² and R¹³ are independently hydrogen, C₁-C₆ alkyl, or C₁-C₆ deuteroalkyl.

11. The compound of any one of claims 1-10, or a pharmaceutically acceptable salt, solvate, or stereoisomer thereof, wherein:
R¹² and R¹³ are independently C₁-C₆ alkyl.

12. The compound of any one of claims 1-11, or a pharmaceutically acceptable salt, solvate, or stereoisomer thereof, wherein:
R¹⁵ and R¹⁸ are independently hydrogen, C₁-C₆ alkyl, or C₁-C₆ deuteroalkyl.

13. The compound of any one of claims 1-12, or a pharmaceutically acceptable salt, solvate, or stereoisomer thereof, wherein:
R¹⁵ and R¹⁸ are independently C₁-C₆ alkyl.

14. The compound of any one of claims 1-13, or a pharmaceutically acceptable salt, solvate, or stereoisomer thereof, wherein:
R³ is —P(═O)(R⁴)₂, —P(═O)(OR⁵)₂, —Y(C₁-C₆alkylene)P(═O)(R⁴)₂, —Y(C₁-C₆alkylene)P(═O)(OR⁵)₂, —B(OR⁵)₂, —Y(C₁-C₆alkylene)B(OR⁵)₂, —S(═O)R⁴, —S(═O)2R⁴, —Y(C₁-C₆alkylene)S(═O)R⁴, —Y(C₁-C₆alkylene)S(═O)₂R⁴, —NR⁵C(═NR^{x})R⁵, —NR⁵C(═NR^{x})NR⁶R⁷, —Y(C₁-C₆alkylene)NR⁵C(═NR^{x})R⁵, —Y(C₁-C₆alkylene)NR⁵C(═NR^{x})NR⁶R⁷, —NR⁵C(═O)(C₁-C₆alkylene)S(═NR^{x})NR⁶R⁷, —NR⁵C(═O)(C₁-C₆alkylene)S(═NR^{x})R⁵, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, —Y(C₁-C₆alkylene)S(═O)(═NR^{x})R⁵, —Y(C₁-C₆alkylene)S(═O)(═NR^{x})NR⁶R⁷, —Y(C₁-C₆alkylene)NR⁵S(═O)₂NR⁵C(═O)R⁵, —Y(C₁-C₆alkylene)NR⁵S(═O)(═NR^{x})R⁵, a N-linked heterocycloalkyl, or a N-linked heteroaryl.

15. The compound of any one of claims 1-13, or a pharmaceutically acceptable salt, solvate, or stereoisomer thereof, wherein:
R³ is —P(═O)(R⁴)₂, —P(═O)(OR⁵)₂, —Y(C₁-C₆alkylene)P(═O)(R⁴)₂, —Y(C₁-C₆alkylene)P(═O)(OR⁵)₂, —B(OR⁵)₂, —Y(C₁-C₆alkylene)B(OR⁵)₂, —S(═O)₂R⁴, —S(═O)₂R⁴, —Y(C₁-C₆alkylene)S(═O)R⁴, —Y(C₁-C₆alkylene)S(═O)₂NR⁴, —NR⁵C(═NR^{x})R⁵, —NR⁵C(═NR^{x})NR⁶R⁷, —Y(C₁-C₆alkylene)NR⁵C(═NR^{x})R⁵, —Y(C₁-C₆alkylene)NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, —Y(C₁-C₆alkylene)S(═O)(═NR^{x})R⁵, —Y(C₁-C₆alkylene)S(═O)(═NR^{x})NR⁶R⁷, —Y(C₁-C₆alkylene)NR⁵S(═O)₂NR⁵C(═O)R⁵, —Y(C₁-C₆alkylene)NR⁵S(═O)(═NR^{x})R⁵, a N-linked heterocycloalkyl, or a N-linked heteroaryl.

16. The compound of any one of claims 1-13, or a pharmaceutically acceptable salt, solvate, or stereoisomer thereof, wherein:
R³ is —P(═O)(R⁴)₂, —P(═O)(OR⁵)₂, —Y(C₁-C₆alkylene)P(═O)(R⁴)₂, or —Y(C₁-C₆alkylene)P(═O)(OR⁵)₂.

17. The compound of any one of claims 1-13, or a pharmaceutically acceptable salt, solvate, or stereoisomer thereof, wherein:
R³ is —B(OR⁵)₂ or —Y(C₁-C₆alkylene)B(OR⁵)₂.

18. The compound of any one of claims 1-13, or a pharmaceutically acceptable salt, solvate, or stereoisomer thereof, wherein:
R³ is —S(═O)R⁴, —S(═O)₂R⁴, —Y(C₁-C₆alkylene)S(═O)R⁴, or —Y(C₁-C₆alkylene)S(═O)₂R⁴.

19. The compound of any one of claims 1-13, or a pharmaceutically acceptable salt, solvate, or stereoisomer thereof, wherein:
R³ is —NR⁵C(═NR^{x})R⁵, —NR⁵C(═NR^{x})NR⁶R⁷, —Y(C₁-C₆alkylene)NR⁵C(═NR^{x})R⁵, or —Y(C₁-C₆alkylene)NR⁵C(═NR^{x})NR⁶R⁷.

20. The compound of any one of claims 1-13, or a pharmaceutically acceptable salt, solvate, or stereoisomer thereof, wherein:
R³ is —Y(C₁-C₆alkylene)NR⁵C(═NR^{x})R⁵ or —Y(C₁-C₆alkylene)NR⁵C(═NR^{x})NR⁶R⁷.

21. The compound of any one of claims 1-13, or a pharmaceutically acceptable salt, solvate, or stereoisomer thereof, wherein:
R³ is —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, —Y(C₁-C₆alkylene)S(═O)(═NR^{x})R⁵, —Y(C₁-C₆alkylene)S(═O)(═NR^{x})NR⁶R⁷, —Y(C₁-C₆alkylene)NR⁵S(═O)₂NR⁵C(═O)R⁵, or —Y(C₁-C₆alkylene)NR⁵S(═O)(═NR^{x})R⁵.

22. The compound of any one of claims 1-13, or a pharmaceutically acceptable salt, solvate, or stereoisomer thereof, wherein:
R³ is —NR⁵C(═O)(C₁-C₆alkylene)S(═NR^{x})NR⁶R⁷ or —NR⁵C(═O)(C₁-C₆alkylene)S(═NR^{x})R⁵.

23. The compound of any one of claims 1-13, or a pharmaceutically acceptable salt, solvate, or stereoisomer thereof, wherein:
R³ is an N-linked heterocycloalkyl or an N-linked heteroaryl.

24. The compound of any one of claims 1-21, or a pharmaceutically acceptable salt, solvate, or stereoisomer thereof, wherein:
Y is a bond.

25. The compound of any one of claims 1-24, or a pharmaceutically acceptable salt, solvate, or stereoisomer thereof, wherein:
R^{X} is —CN.

26. The compound of any one of claims 1-25, or a pharmaceutically acceptable salt, solvate, or stereoisomer thereof, wherein:
each R⁴ is independently C₁-C₆ alkyl, C₁-C₆ haloalkyl, or C₁-C₆ deuteroalkyl.

27. The compound of any one of claims 1-26, or a pharmaceutically acceptable salt, solvate, or stereoisomer thereof, wherein:
each R⁵ is independently hydrogen, C₁-C₆ alkyl, C₁-C₆ haloalkyl, or C₁-C₆ deuteroalkyl.

28. The compound of any one of claims 1-27, or a pharmaceutically acceptable salt, solvate, or stereoisomer thereof, wherein:
two R⁵ are taken together to form an optionally substituted heterocycloalkyl.

29. The compound of any one of claims 1-28, or a pharmaceutically acceptable salt, solvate, or stereoisomer thereof, wherein:
each R⁶ and R⁷ is independently hydrogen, C₁-C₆ alkyl, C₁-C₆ haloalkyl, or C₁-C₆ deuteroalkyl.

30. The compound of any one of claim 1, or a pharmaceutically acceptable salt, solvate, or stereoisomer thereof, wherein the compound is: 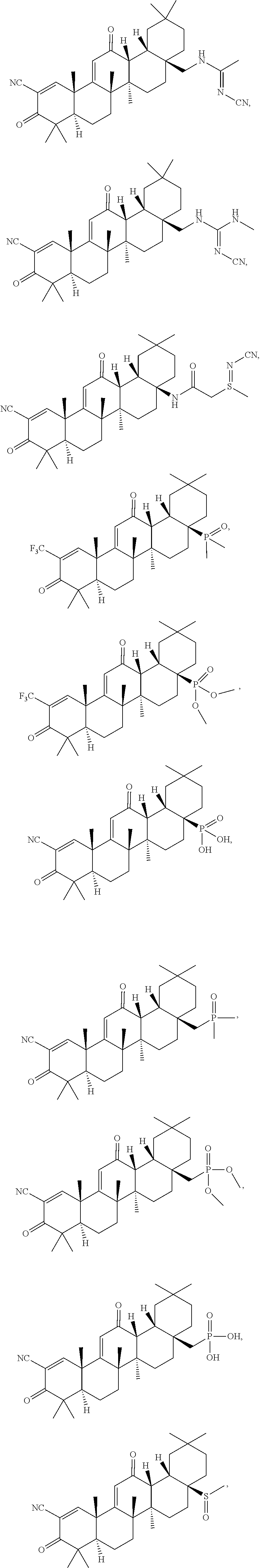 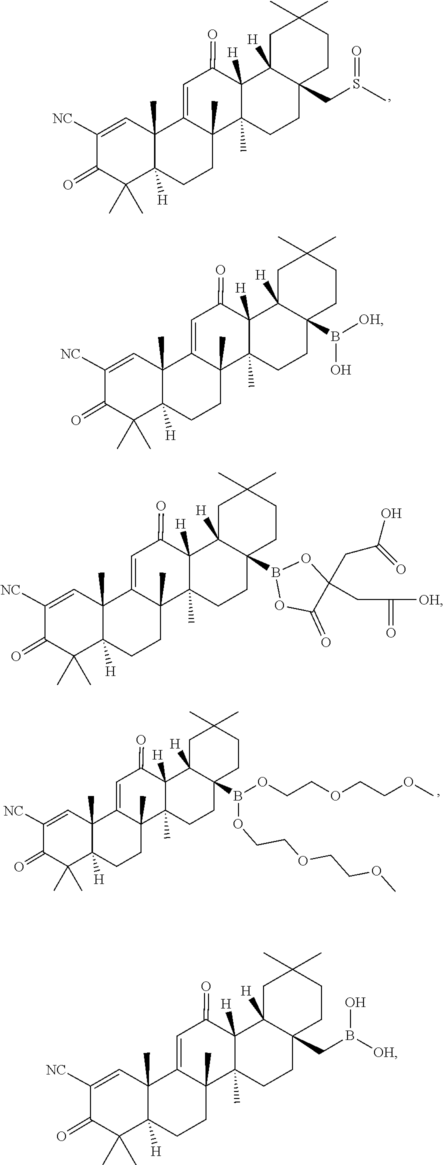 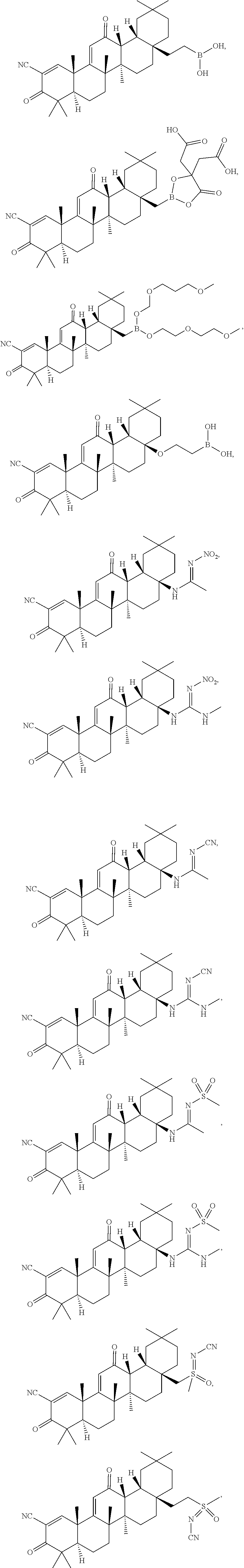 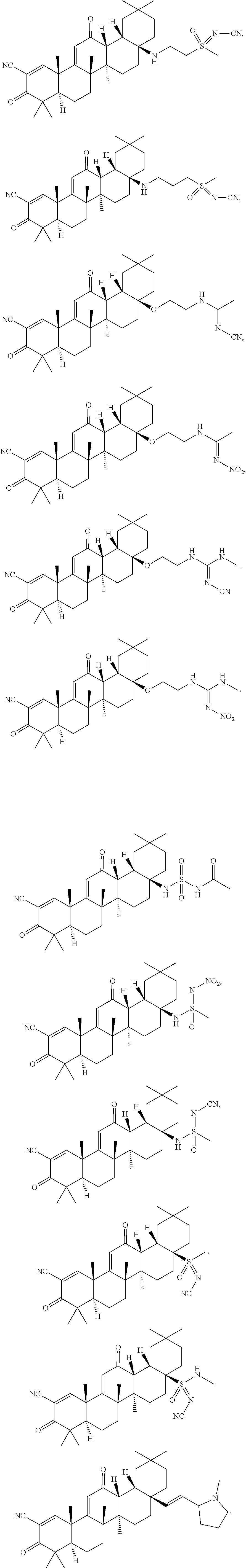 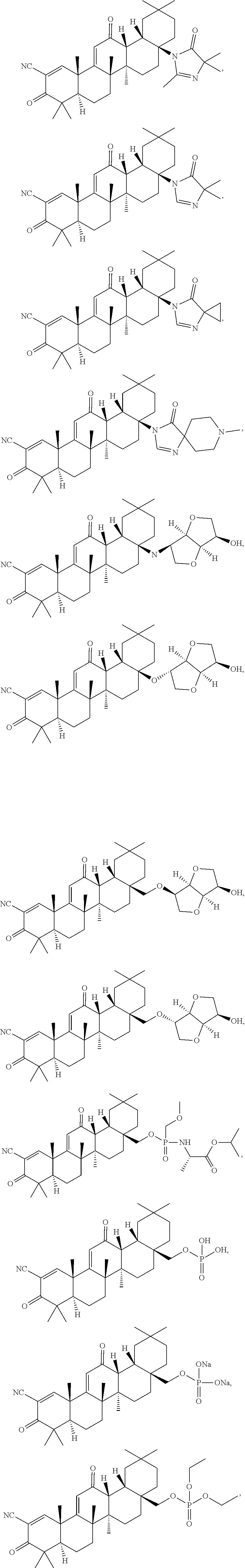 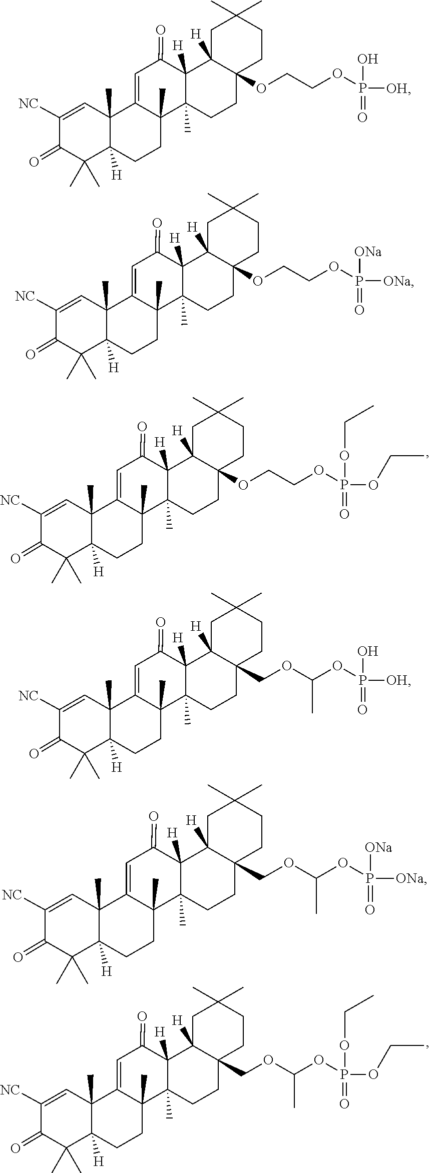
or.

31. A compound of Formula (VIII), or a pharmaceutically acceptable salt, solvate, or stereoisomer thereof: 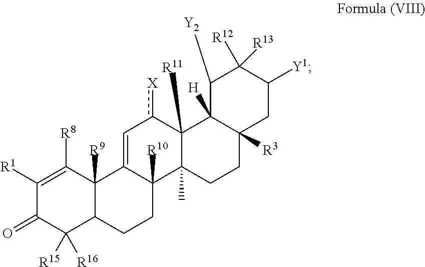
wherein:
R¹ is hydrogen, deuterium, halogen, —CN, —OR^{b}, —NR^{c}R^{d}, —NR^{b}S(═O)₂R^{a}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R³ is halogen, —CN, —S(═O)R⁴, —S(═O)₂R⁴, —NO₂, —NR⁶R⁷, —S(═O)2NR⁶R⁷, —C(═O)R⁴, —OC(═O)R⁴, —C(═O)OR⁵, —OC(═O)OR⁵, —C(═O)NR⁶R⁷, -OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3a};
each R^{3a} is independently oxo, deuterium, halogen, —CN, -01V, —S(═O)R⁴, —S(═O)₂R⁴, —NO₂, —NR⁶R⁷, —S(═O)₂NR⁶R⁷, —C(═O)R⁴, —OC(═O)R⁴, —C(═O)01e, —OC(═O)08⁵, —C(═O)NR⁶R⁷, —OC(═O)NR⁶R⁷, —NR⁵C(═O)NR⁶R⁷, —NR⁵C(═O)OR⁵, —NR⁵S(═O)₂NR⁶R⁷, —NR⁵S(═O)₂R⁴, —NR⁵C(═O)R⁴, —P(═O)(R⁴)₂, —P(═O)(OR⁵)₂, —B(OR⁵)₂, —NR⁵C(═NR^{x})R⁵, —NR⁵C(═NR^{x})NR⁶R⁷, —S(═O)(═NR^{x})R⁵, —S(═O)(═NR^{x})NR⁶R⁷, —NR⁵S(═O)₂NR⁵C(═O)R⁵, —NR⁵S(═O)(═NR^{x})R⁵, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{3b};
each R^{3b} is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R^{x} is hydrogen, —NO₂, —CN, or —S(═O)₂R^{a};
each R⁴ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{4a};
each R^{4a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁵ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{5a};
or two R⁵ are taken together to form an optionally substituted heterocycloalkyl;
each R^{5a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R⁶ and R⁷ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{6a};
each R^{6a} is independently oxo, deuterium, halogen, —CN, —OR^{b}, —NR^{c}R^{d}, —C(═O)R^{a}, —C(═O)OR^{b}, —C(═O)NR^{c}R^{d}, —NR^{b}C(═NR^{x})R^{b}, —NR^{b}C(═NR^{x})NR^{c}R^{d}, —S(═O)(═NR^{x})R^{b}, —S(═O)(═NR^{x})NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R⁶ and R⁷ are taken together with the nitrogen atom to which they are attached to form a heterocycloalkyl optionally substituted with one, two, or three R^{6b};
each R^{6b} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; 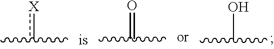
Y¹ and Y² are independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, or C₁-C₆ heteroalkyl;
R⁸ is hydrogen, deuterium, halogen, —CN, —OR^{b}, —NR^{c}R^{d}, —C(═O)R^{a}, —C(═O)OR^{b}, C₁-C₆ alkyl, C₂-C₆ alkenyl, C₂-C₆ alkynyl, C₁-C₆ haloalkyl, or C₁-C₆ deuteroalkyl;
R⁹ is C₁-C₆ alkyl, C₂-C₆ alkenyl, C₂-C₆ alkynyl, C₁-C₆ haloalkyl, or C₁-C₆ deuteroalkyl;
R¹⁰ is C₁-C₆ alkyl, C₂-C₆ alkenyl, C₂-C₆ alkynyl, C₁-C₆ haloalkyl, or C₁-C₆ deuteroalkyl;
R¹¹ is hydrogen, deuterium, halogen, or —OR^{b};
or R³ and R⁴ are taken together to form an optionally substituted cycloalkyl or an optionally substituted heterocycloalkyl;
R¹² is hydrogen, deuterium, —OR^{b}, —NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R¹³ is —CN, —OR¹⁹, —S(═O)₂NR²⁰R²¹, —OC(═O)R¹⁸, —OC(═O)OR¹⁹, —OC(═O)NR²⁰R²¹, —NR¹⁹C(═O)OR¹⁹, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, heteroaryl, —CH₂(cycloalkyl), —CH₂(heterocycloalkyl), —CH₂(aryl), or —CH₂(heteroaryl); wherein the alkyl, cycloalkyl, heterocycloalkyl, aryl, and heteroaryl are independently optionally substituted with one, two, or three R¹³a;
each R^{13a} is independently oxo, deuterium, halogen, —CN, —OR¹⁹, —SR¹⁹, —S(═O)R¹⁸, —S(═O)₂R¹⁸, —NO₂, —NR²⁰R²¹, —S(═O)₂NR²⁰R²¹, —C(═O)R¹⁸, —OC(═O)R¹⁸, —C(═O)OR¹⁹, —OC(═O)OR¹⁹, —C(═O)NR²⁰R²¹, —OC(═O)NR²⁰R²¹, —NR¹⁹C(═O)OR¹⁹, C₁-C₆ alkyl, C₂-C₆ alkenyl, C₂-C₆ alkynyl, C₁-C₆ haloalkyl, C₁- C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R¹⁸ is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{18a};
each R^{18a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C_{I}—C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R¹⁹ is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{19a};
each R^{19a} is independently oxo, deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
each R²⁰ and R²¹ are independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three R^{20a};
each R^{20a} is independently oxo, deuterium, halogen, —CN, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
or R²⁰ and R²¹ are taken together with the nitrogen atom to which they are attached to form a heterocycloalkyl optionally substituted with one, two, or three R^{20b};
each R^{20b} is independently oxo, deuterium, halogen, —CN, —OR^{b}, 13 NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl;
R¹⁵ and R¹⁶ are independently hydrogen, —OR^{b}, —NR^{c}R^{d}, C₁-C₆ alkyl, C₂-C₆ alkenyl, C₂-C₆ alkynyl, C₁- C₆ haloalkyl, C₁-C₆ deuteroalkyl, C₁-C₆ hydroxyalkyl, C₁-C₆ aminoalkyl, or C₁-C₆ heteroalkyl;
or R¹⁵ and R¹⁶ are taken together to form a cycloalkyl or a heterocycloalkyl; each substituted with 0-6 R²;
each R² is independently deuterium, halogen, —CN, —OR^{b}, —NR^{c}R^{d}, —C(═O)R^{a}, —C(═O)OR^{b}, —C(═O)NR^{c}R^{d}, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, or heterocycloalkyl;
each R^{a} is independently C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
each R^{b} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three halogen, —OH, —NH₂, or C₁-C₆ alkyl; and
each R^{c} and R^{d} is independently hydrogen, C₁-C₆ alkyl, C₂-C₆ alkenyl, C₂-C₆ alkynyl, C₁-C₆ haloalkyl, C₁-C₆ deuteroalkyl, C₁-C₆ hydroxyalkyl, C₁-C₆ aminoalkyl, C₁-C₆ heteroalkyl, cycloalkyl, heterocycloalkyl, aryl, or heteroaryl; wherein the alkyl, alkenyl, alkynyl, cycloalkyl, heterocycloalkyl, aryl, and heteroaryl are independently optionally substituted with one, two, or three deuterium, halogen, —OH, —NH₂, or C₁-C₆ alkyl;
or R^{c} and R^{d} are taken together with the nitrogen atom to which they are attached to form a heterocycloalkyl optionally substituted with one, two, or three deuterium, halogen, —OH, —NH₂, or C₁-C₆ alkyl.

32. The compound of claim 31, or a pharmaceutically acceptable salt, solvate, or stereoisomer thereof, wherein:
R¹ is —CN, C₁-C₆ alkyl, C₁-C₆ haloalkyl, or C₁-C₆ deuteroalkyl.

33. The compound of claim 31 or 32, or a pharmaceutically acceptable salt, solvate, or stereoisomer thereof, wherein:
R¹ is —CN.

34. The compound of any one of claims 31-33, or a pharmaceutically acceptable salt, solvate, or stereoisomer thereof, wherein:
R⁸ is hydrogen.

35. The compound of any one of claims 31-34, or a pharmaceutically acceptable salt, solvate, or stereoisomer thereof, wherein:
R⁹ is C₁-C₆ alkyl.

36. The compound of any one of claims 31-35, or a pharmaceutically acceptable salt, solvate, or stereoisomer thereof, wherein:
R¹⁰ is C₁-C₆ alkyl.

37. The compound of any one of claims 31-36, or a pharmaceutically acceptable salt, solvate, or stereoisomer thereof, wherein:

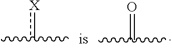

38. The compound of any one of claims 31-37, or a pharmaceutically acceptable salt, solvate, or stereoisomer thereof, wherein:
Y¹ and Y² are hydrogen.

39. The compound of any one of claims 31-38, or a pharmaceutically acceptable salt, solvate, or stereoisomer thereof, wherein:
R¹¹ is hydrogen, halogen, or —OH.

40. The compound of any one of claims 31-39, or a pharmaceutically acceptable salt, solvate, or stereoisomer thereof, wherein:
R¹¹ is hydrogen.

41. The compound of any one of claims 31-40, or a pharmaceutically acceptable salt, solvate, or stereoisomer thereof, wherein:
R¹² is hydrogen, C₁-C₆ alkyl, or C₁-C₆ deuteroalkyl.

42. The compound of any one of claims 31-41, or a pharmaceutically acceptable salt, solvate, or stereoisomer thereof, wherein:
R¹² is C6 alkyl.

43. The compound of any one of claims 31-42, or a pharmaceutically acceptable salt, solvate, or stereoisomer thereof, wherein:
R¹³ is —CN, heterocycloalkyl, heteroaryl, or —CH₂(heteroaryl); wherein the heterocycloalkyl and heteroaryl are independently optionally substituted with one, two, or three R^{13a}.

44. The compound of any one of claims 31-43, or a pharmaceutically acceptable salt, solvate, or stereoisomer thereof, wherein:
R¹³ is heterocycloalkyl or heteroaryl; wherein the heterocycloalkyl and heteroaryl are independently optionally substituted with one, two, or three R^{13a}.

45. The compound of any one of claims 31-44, or a pharmaceutically acceptable salt, solvate, or stereoisomer thereof, wherein:
R¹³ is heteroaryl optionally substituted with one, two, or three R^{13a}.

46. The compound of any one of claims 31-45, or a pharmaceutically acceptable salt, solvate, or stereoisomer thereof, wherein:
R¹³ is heterocycloalkyl optionally substituted with one, two, or three R^{13a}.

47. The compound of any one of claims 31-46, or a pharmaceutically acceptable salt, solvate, or stereoisomer thereof, wherein:
each R^{13a} is independently oxo, —OR¹⁹, —NR²⁰R²¹, —C(═O)OR¹⁹, —C(═O)NR²⁰R²¹ , C₁-C₆ alkyl, C₁-C₆ haloalkyl, C₁-C₆ deuteroalkyl, C₁-C₆ hydroxyalkyl, C₁-C₆ aminoalkyl, C₁-C₆ heteroalkyl, or aryl.

48. The compound of any one of claims 31-47, or a pharmaceutically acceptable salt, solvate, or stereoisomer thereof, wherein:
each R^{13a} is independently oxo, —OR¹⁹, —NR²⁰R²¹, —C(═O)NR²⁰R²¹, C₁-C₆ alkyl, C₁-C₆ haloalkyl, C₁-C₆ hydroxyalkyl, or aryl.

49. The compound of any one of claims 31-48, or a pharmaceutically acceptable salt, solvate, or stereoisomer thereof, wherein:
each R^{13a} is independently —OR¹⁹, —NR²⁰R²¹, or —C(═O)NR²⁰R²¹.

50. The compound of any one of claims 31-49, or a pharmaceutically acceptable salt, solvate, or stereoisomer thereof, wherein:
R¹⁵ and R¹⁶ are independently hydrogen, C₁-C₆ alkyl, or C₁-C₆ deuteroalkyl.

51. The compound of any one of claims 31-50, or a pharmaceutically acceptable salt, solvate, or stereoisomer thereof, wherein:
R¹⁵ and R¹⁶ are independently C₁-C₆ alkyl.

52. The compound of any one of claims 31-51, or a pharmaceutically acceptable salt, solvate, or stereoisomer thereof, wherein:
R³ is C₁-C₆ alkyl or —C(═O)OR⁵.

53. The compound of any one of claims 31-52, or a pharmaceutically acceptable salt, solvate, or stereoisomer thereof, wherein:
R³ is C₁-C₆ alkyl.

54. The compound of claim 31, or a pharmaceutically acceptable salt, solvate, or stereoisomer thereof, wherein the compound is:

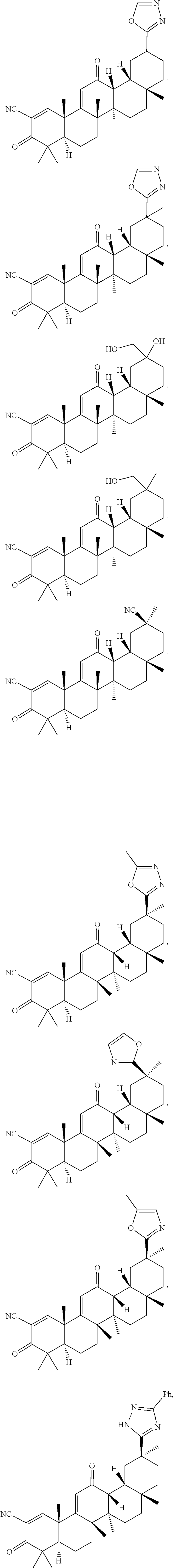

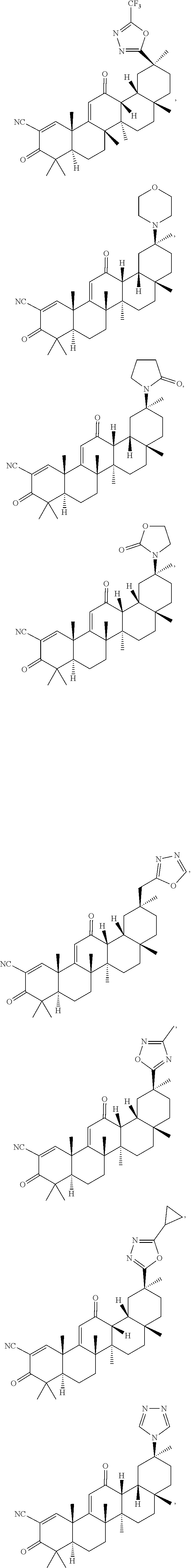

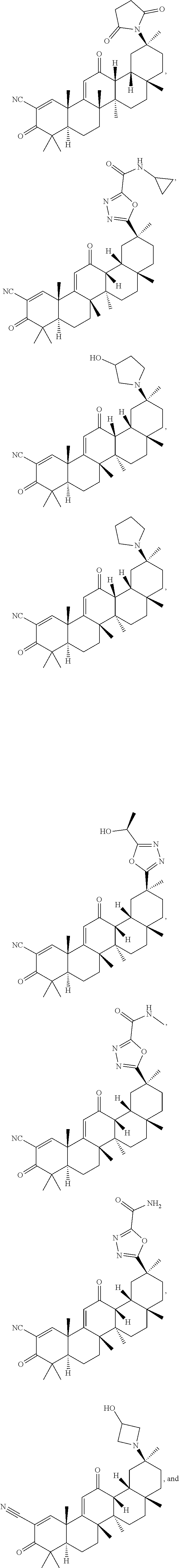

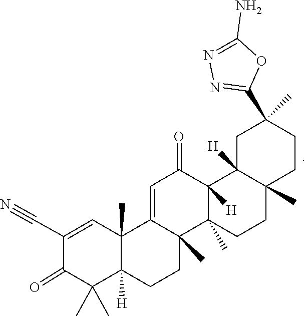

55. A pharmaceutical composition comprising a therapeutically effective amount of the compound of any one of claims 1-54, and a pharmaceutically acceptable excipient.

56. A method for treating a disease in a mammal comprising administering to the mammal a therapeutically effective amount of the compound of any one of claims 1-54, or the pharmaceutical composition of claim 55.

57. The method of claim 56, wherein the disease is an inflammatory disease.

58. The method of claim 56, wherein the disease is diabetic nephropathy or chronic kidney disease.

59. The method of claim 56, wherein the disease is chronic obstructive pulmonary disease (COPD) or inflammatory bowel disease (IBD).

60. The method of claim 56, wherein the disease is nonalcoholic steatohepatitis (NASH).
